(19) United States
(12) Patent Application Publication
(10) Pub. No.: US 2019/0064110 A1
(43) Pub. Date: Feb. 28, 2019

(54) A PICOMETER-DIAMETER PORE IN AN INORGANIC MEMBRANE FOR SEQUENCING PROTEIN

(71) Applicant: UNIVERSITY OF NOTRE DAME, Notre Dame, IN (US)

(72) Inventors: Gregory Timp, Notre Dame, IN (US); Eamonn Kennedy, Notre Dame, IN (US); Zhuxin Dong, Notre Dame, IN (US); Clare Tennant, Notre Dame, IN (US)

(21) Appl. No.: 15/770,717

(22) PCT Filed: Oct. 24, 2016
(86) PCT No.: PCT/US2016/058519

§ 371 Date: Apr. 24, 2018

Related U.S. Application Data
(60) Provisional application No. 62/246,015, filed on Oct. 24, 2015.

(51) Int. Cl. G01N 27/447; G01N 33/68

(52) U.S. Cl. CPC G01N 27/44791; G01N 27/44747; G01N 33/6818

(57) ABSTRACT

Disclosed are thin inorganic membranes having a defined topography that includes pores having a defined diameter of nanometer and sub-nanometer diameter. The thin membranes are resistant to protein denaturing agents, and may be employed in analytical and clinical methods for identifying single amino acid residues within the sequence of a protein, and the pores are other than MspA pores. Methods for making a thin inorganic membrane with nanopore and sub-nanopore topography and conical cone structure are also disclosed. The thin inorganic membrane may be comprised of any denaturant-resistant materials, such as silicon nitride. A method for manufacturing the thin inorganic membrane with nanopores is also provided, and provides a thin surface with a defined conical topography, the nanopores being provided on the membrane surface with an electron beam sputtering technique.

### CROSS-REFERENCE TO RELATED APPLICATIONS

The present application claims priority to United States Provisional Patent Application U.S. Ser. No. 62/246,015, filed Oct. 24, 2015. The entire content of U.S. Ser. No. 62/246,015 is specifically incorporated herein by reference.

### STATEMENT REGARDING FEDERALLY SPONSORED RESEARCH OR DEVELOPMENT

The United States government owns rights to the present application as work used to create the invention was supported by grants from the National Science Foundation [DBI 1256052] and the National Institutes of Health (SBIR Phase II, 5R44EB008589-04).

### FIELD OF THE INVENTION

This invention relates to tools, materials and methods useful in the sequencing of biological molecules, such as proteins. In particular, the invention relates to the field of membrane materials, such as membranes made of inorganic materials, having small pores (nanometer, picometer diameter). The invention also relates to the field of protein and peptide sequencing using an inorganic material with small pores.

### BACKGROUND OF THE INVENTION

The primary structure of a protein, which consists of a linear sequence of amino acids (AAs) linked by peptide bonds, essentially dictates how the protein binds to itself and how it functions. Proteins are the machinery that make biology work. The three-dimensional (3D) structure of a protein, which relates to how a protein binds to itself, determines its function. The protein's 3D structure is essentially dictated by the primary structure, which consists of a linear sequence of amino acids (AAs) linked by peptide bonds. Thus, sequencing a protein is essential to proteomics, the next step beyond genomics, in the analysis of biology.

Primarily two methods are used for protein/peptide sequencing, mass spectrometry (MS) and Edman degradation (ED). However, these two methods suffer significant limitations, that, among other things, render them inappropriate for use in clinical applications. For example, ED is not useful for sequencing a peptide where the N-terminal AA is chemically modified or buried in the folded protein, and is limited to use with sequencing peptides about 50 amino acid residues long. While MS can sequence a protein of any size, this sequencing method relies on enzymatic digestion, and becomes increasingly more difficult and computationally demanding to use as the length of the amino acid sequence of the molecule being sequenced increases. This, in part, is because of the increased difficulty associated with reassembling the digested amino acid sequence as the length of the amino acid sequence being analyzed increases. In addition, analysis by MS requires the use of relatively highly concentrated samples of the peptide/protein being sequenced (>fmole/L-scale).

Alternatives to the ED and MS methods to address the above limitations have been proposed. For example, the measurement of single peptides have been reported using electron tunneling, with the measurement of individual amino acid residues of a peptide sequence being read as the amino acid residue passes through a 0.55-0.70 nm gap between electrodes. However, the use of this method for analysis of peptides/proteins having long amino acid length. Successful use of this method to sequence each AA residue within a long amino acid encoding sequence of a polypeptide have not been reported.

The sequencing of biological materials using a method that employs small pores has been used for sequencing nucleic acids (DNA). However, this approach has not been proposed for use in the sequencing of proteins or peptides. Several hurdles exist to the use of a small pores, such as within a thin membrane, for the sequencing of an AA sequence of a protein or peptide, especially for sequencing proteins or peptides with relatively long (greater that 50 AA length) sequence lengths. For example, to sequence a single protein with a pore, several technical hurdles have to be overcome. First, the protein has to be denatured to eliminate the higher-order structure and facilitate the interpretation of the blockage current associated with single AAs. Second, the deficient chemical sensitivity of a pore, (which can be related to the volume occluded by a molecule such as an amino acid), the charge distribution and the dependence of the monomer (e.g., AA) mobility, create technical barriers to successful AA sequencing. In addition, the background noise associated with reading a particular AA, must be better controlled and/or mitigated to improve accuracy of the sequence reading. Third, the use of a membrane with pores for AA sequencing requires the use of an electrical field to drive the AA through the pore. However, if an electric force field in the pore is to be used to systematically drive a molecule (AA) through a pore, the charge distribution along the protein AA sequence must be uniform. Improved techniques for maintaining the charge uniformity along a protein AA sequence are needed in order to employ this approach to AA sequencing of a protein/peptide.

A need continues to exist in the arts of analytical processes and materials useful; therein for improved materials and techniques that overcome these and other limitations.

### SUMMARY OF THE INVENTION

In a general and overall sense, the present invention presents materials and highly sensitive and accurate methods that provide for the sequencing of a single amino acid, as well as short strings of four (4) amino acids, within an amino acid sequence encoding a molecule of interest, such as a protein or antibody.

Inorganic Membrane Materials:

In some aspects, the invention provides thin membranes of inorganic materials (such as silicon nitride), having superior abilities for sequencing single AA's and short AA sequences (such as a quadromer (four amino acids)), within an amino acid sequence encoding a protein/peptide containing molecule of interest, such as a protein or peptide. By way of example, a protein/peptide may comprise an antibody (such as a polyclonal or monoclonal antibody (e.g., IgG), protein (such as H3.3), or other molecule having either a native or non-native amino acid sequence.

The inorganic material of the thin membrane will comprise a material that is relatively resistant to denaturing agents, such as SDS. Because the thin membranes are envisioned to be used with samples and under conditions that may expose the membrane to denaturing materials, the thin membranes will not comprise materials that will become denatured or otherwise become compromised in the presence of a denaturing material, such as SDS or other detergent. By way of example and not limitation, one such material that may be employed in the fabrication to the thin membranes of the invention is silicon nitride. It is anticipated that virtually any other material that is capable of being provided in a thin film configuration, and that is suitable of being processed to include the nanopores and/or picopores described herein, and/or that is further amenable to electron beam sputtering techniques to provide said picopores and/or nanopores on the surface, may be used in the practice of the present invention.

In some embodiments, the inorganic membrane comprises a surface having a defined topography and nanometer (nm) (eg, “nanopores”) and/or sub-nanometer (e.g., “sub-nanopores” or “picopores”) size pores. In particular embodiments, the nanopores have a pore size of about 1.5 nm, 0.7 nm, and 0.3 nm. In other embodiments, the sub-nanopores are described as having a pore size of less than 1,000 pm. In particular embodiments, the picopores and/or sub-nanopores have a pore size of about 300 pm to about 700 pm in diameter. In particular embodiments, the picopores and/or sub-nanopore will have a pore size of about 500 pm.

The thin membrane comprising the nanopores and/or picopores may include nanopores and/or picopores covering the entire surface of the membrane, or less than all of the surface of the membrane. For example, the surface may comprise anywhere from 25% surface area, 50% surface area, 75% of the surface area to up to 100% of the surface area of the inorganic membrane to include the nanopores and/or picopores of the present invention/

The nanopores and/or picopores are described herein as other than a biological pore. For example, an MspA pore is considered a biological pore. These types of pores are less useful in the practice of the sequencing amino acids with the present materials and methods. Therefore, in certain embodiments, the pores are not MspA pores, or are not biological pores.

The thin inorganic membrane, in some embodiments, may be described as having a thickness of about t=8 nm to 12 nm.

In some embodiments, the inorganic thin membranes of the invention may be described as having a defined and distinction topography that may be described as a biconal configuration. The biconal confirmation relates to the configuration of the nanopores and/or picopores on the surface of the membrane. The biconal configuration of the pores, in some embodiments, may be described as having cone angles of about cone angles in a range of about θ=15+/−5°.

The picopores and/or nanopores of the membrane may be further described as having a size that is smaller than the secondary structure of a protein sequence of interest to be sequenced, and as having a cross-section near the “waist” (i.e., middle region) that is comparable to the size of a hydrated ion. These features are expected to enhance the chemical specificity of the sequencing of the protein, especially in the sequencing of an antibody.

The nanopores and/or picopores may also be described as having a size that is smaller than the size of an α-helix (which has a diameter of <−0.5 and a rise of 0.56 nm). The α-helix is a characteristic of the secondary structure found in a protein. Therefore, this size characteristic is a feature of the nanopores and picopores of the membranes that provides the enhanced chemical specificity in amino acid sequencing achieved in the use of these membranes.

The nanopores and/or picopores are provided onto a thin inorganic membrane via an electron beam sputtering technique. Specifically, pores with sub-nanometer cross-sections may be sputtered through a thin membrane, such as a silicon nitride membrane, using a tight, high-energy electron beam carrying a current ranging from 300-500 pA (especially 398 pA) (post alignment) in a scanning transmission electron microscope with a Super-TWIN pole piece and a 0.3 nm diameter pore, in 50 seconds.

Methods of Sequencing Biological Materials: Single Amino Acids and/or Short Length Amino Acid Sequences (Quadromers)

In another aspect, the invention provides methods for sequencing biological materials, such as proteins.

In some embodiments, the method provides a sequencing method comprising sequencing an amino acid in a protein molecule, such as an antibody (e.g., IgG) or other protein. In particular embodiments, the method comprises a first step of denaturing a protein of interest. In some embodiments, the protein is denatured with sodium dodecyl sulfate (SDS) prior to sequencing according to the present methods.

In the second step, the denatured protein is applied to a thin inorganic membrane comprising a nanopore and/or picopore (such as a nanopore having a size of between about 300-700 pm in diameter) surface, said pores having a defined conical topography, wherein amino acid residues of the denatured protein sequence become associated with the pores of the inorganic membrane. The inorganic membrane (having associated on its surface the amino acid sequence of the denatured protein), is then immersed in an electrolyte solution, such as NaCl (such as a 200-300 mM NaCl solution), and allowed to stand for a period of time (such as 24 hours) sufficient to provide sufficient “wetting” of the membrane. An electronic current is then applied to this “wetted” membrane. The fluxuations of the electronic current are to be recorded.

During the step when an electric current is applied to the “wetted” surface, the electronic current will be applied so as to achieve nearly regular picoAmpere current fluctuations at the membrane surface. The amino acids of the denatured protein impelled through the picopore and/or nanopore of the membrane will be correlated with the fluxuation in the electronic current observed at the point the amino acid is impelled out of the pore. Therefore, the amplitudes of the electronic current fluxuations observed when the electric current is applied to the membrane surface, and the amino acid sequence is impelled through the nanopore, will be recorded as part of the method. The current fluxuations attendant the impelling of the amino acid through a pore is used to identify an amino acid sequence for the amino acid that was impelled.

Each sub-nanopore (nm) and/or picopore (pm) diameter sized pore) in the inorganic membrane surface permits a single amino acid residue to pass there through upon application of an appropriate voltage current to the membrane in the presence of an electrolyte solution.

It has been demonstrated in the present methods that the number of nanopores and/or picopores on the inorganic membrane surface coincides and/or provides a useful quantitative correlation with the number of amino acid residues of the protein or other molecule being sequenced. It has been found that the amplitudes of the fluctuations are highly correlated with the volume occluded at the “waist” of the nanopore/picopore, by a 3-5 AA sequence. Considering that a single protein of any molecular weight could be sequenced with a picopore, the present methodologies augment and/or supplant the short reads limitations of mass spectrometry (MS), and replace them with long reads needed for quantitation.

The methods may be used in the sequencing of amino acid of a protein, such as an antibody, including both monoclonal antibodies and polyclonal antibodies. In addition, other proteins having a length of about 4 to about 300 amino acids, or more.

The method employs a thin inorganic membrane having a surface comprising nanometer (nanopores) and/or picometer sized pores (picopores)(i.e., less than 1,000 picometers, such as picopores with a diameter of about 300-700 pm in diameter), for sequencing individual amino acids and/or very short lengths (e.g., quadromers (four amino acids)) of amino acids in a single protein molecule. The thin membranes having nanometer, sub-nanometer and picometer sized pores provided herein are demonstrated to detect and analyze polypeptides and proteins in pure solutions by measuring fluxuations in electronic current associated with the impelling of a particular amino acid through a pore of the membrane. “Noise” associated with the process (blockade current) may also be assessed to enhance the sensitivity and accuracy of the method results.

The present invention provides:

1. The use of a membrane having pores comparable in size to a native protein to examine amino acid sequence domains that are captured in the lumen of a membrane without translocating through it;

2. The amino acid sequencing of native proteins by a methodology that employs an inorganic membrane having pores large enough (>10 nm) to allow translocation of the amino acid through the membrane; and

3. Measurements of single polymers translocating through a small pore lumen. The secondary and higher order structure of the protein confounds the interpretation of the blockage current, and the charge distribution along the native protein is not uniform. These two problems are overcome by the present invention, which provides a systematic control of the translocation kinetics of the electric field in a small pore.

Method of Making a Thin Inorganic Membrane Having a Conical Topography and Nanometer and Subnanometer Sized Pores/Methods of Fabricating a Thin Inorganic Membrane:

In another aspect, the invention provides for a method for preparing an inorganic membrane using an electronic beam sputtering technique that provides nanopores and/or picopores on a thin inorganic membrane. This method provides a thin film that may be used in a technique for sequencing of a single amino acid. The exquisite control exercised over the pore topography by electron beam-induced sputtering in a scanning transmission electron microscope (STEM), in combination with ultra-thin inorganic membranes, affords provides the capacity to make pores smaller than the size of an α-helix (which has a diameter <500 pm and a rise of 560 pm), a common secondary structure found in a protein, and comparable to the size of a hydrated ion (which has a mean distance between oxygen atoms in the water molecules in the first hydration shell surrounding a sodium ion of about 210 pm.).

The denaturing step of the protein is accomplished using a combination of an anionic detergent, heat and a reducing agent. In particular, SDS is used as the anionic detergent, in combination with heat (45-100° C.) and reducing agents like BME to impart a nearly uniform negative charge to the protein, stabilizing denaturation. The uniform charge offers the extra-added benefit of facilitating electrical control of the translocation kinetics.

The chemical specificity of a picopore is due in part to its volume, which is comparable to the volume of an amino acid (AA) residue and the size of a hydrated ion. When it is immersed in electrolyte and an electric field is impelled through the picopore, which coincide precisely with the number of residues in the protein. Furthermore, the amplitudes of the fluctuations are highly correlated with the volume occluded by the 3-5 AAs located in the waist of the picopore. Thus, the blockage current trace likely reflects a moving average of the occluded volumes associated with no fewer than three AAs. Multiple monomers affecting the blockade current like this do not really pose a problem for sequencing, however, so long as the translocation rate is controlled—i.e. a Viterbi algorithm might be adapted to untangle the sequence with single AA resolution even if the signal is noisy.

Noise may play a beneficial role in detection of AAs in a picopore by enhancing the detection of the relatively weak blockade signals that convey information about the AAs. Noise was omnipresent in the pore current. The low frequency power spectral density S_{l/f} in a picopore was also found to be inversely proportional to the frequency with an amplitude that depends on the inverse square of the open pore current I²₀ at low current. However, for higher currents in pores with cross-sections smaller than 0.41 nm², S_{l/f} was observed to be independent of the current, which signals the development of correlations in the current fluctuations. Such correlations in the noise have been attributed to a (traffic) “jamming” transition in which congestion between the carriers develops at high current, giving rise to non-linear density waves and bunching of the ions in the pore. Consistent with this attribution, as the picopore cross-section is reduced, the threshold current for the jamming transition reduces dramatically, from I=70 pA for 0.21 nm² to I−1.5 pA at 0.12 nm² whereas no threshold was observed for pores with a cross-section ≥0.41 mn². Taken altogether, these data support the contention that the waist of the picopores were comparable to the size of hydrated sodium ions.

In nonlinear systems with a threshold, noise can enhance detection through stochastic resonance. It is postulated here that excess noise contributed by correlations in the ionic blockade current due to the occluded residue volume enhanced the fractional blockade. While not intending to be limited to any particular mechanism of action, the small size of the waist of the picopore may affect the chemical sensitivity in two ways. First, the signal associated with each residue is improved because the occluded volume is comparable to the effected pore volume. Second, a diameter this small is expected to introduce correlations in the ion transport that affect the electrical noise, increasing it when the blockaded pore current exceeds the jamming threshold.

The following presents a brief description of the figures provided in the present disclosure.

### BRIEF DESCRIPTION OF THE DRAWINGS

FIG. 1a-1f. Detecting single protein molecules using a pore with a sub-nanometer cross-section (1a). Schematic representation of the apparatus used to measure the force and current associated with protein in a sub-nanopore. A sub-nanopore in a silicon nitride membrane is embedded in a two-layer (cis/trans) microfluidic device made from PDMS. An electrical bias is applied between a Ag/AgCl electrode embedded in the trans-channel and the current is measured using an amplifier connected directly to the cantilever holder. (1b) Cutaway of the schematic showing biotinylated H3.2 tethered to the tip of an A FM cantilever through a bond to streptavidin, translocating through the pore. (1c, 1d; 1i) TEM micrographs of pores with nominal diameters of 1 nm; and 0.6 nm respectively, sputtered through silicon nitride membranes that are about 10 nm thick. The shot noise is associated with electron transmission through the pore. (1c, 1d; ii) Multislice simulations of the TEM images consistent with the experimental conditions. The simulations correspond to bi-conical pores with a 1.0×1.3 nm2 and 0.6×0.7 nm2 cross-sections and a 200 cone angle at defocus of −40 nm. The close correspondence between the simulations and the actual TEM images signified that the models accurately reflected the actual pore structure. (1c, 1d; iii) Two-dimensional projections from the top through the model showing the atomic distribution near the pore waist. The atoms are represented by a space-filling model in which Si is a sphere with a 0.235 nm diameter and N is a sphere with a 0.13 nm diameter. (1e). Concomitant direct measurements of the force and current as H3.2 protein was impelled through a pore with a 1.0×1.3 nm² cross-section. The force (top) on protein and the ionic blockade current (bottom) under an applied potential of 0.7 V measured while the A FM cantilever was retracted from the pore at 4.0 nm/s in 200 mM NaCl, showing a relatively frictionless plateau in the force. Inset: The cartoon shows the assumed molecular configuration with the arrow indicating the direction of the cantilever motion. (1f) Like FIG. 1(e), the force (top) and current blockade (bottom) observed under the same conditions showing a single H3.2 molecule stretching in the pore, producing a force-extension curve reflecting the molecular elasticity. The blue line represents a fit to the FJC model for the stretch.

FIG. 2a-2h. Forces and currents associated with a single H3.2 protein molecule sliding frictionlessly through a sub-nanopore with a 0.6×0.7 nm² cross-section. (2a) The force (top), a magnified view of the force (middle), and the blockade current (bottom) measured as H3.2 is extracted at 4.0 nm/s from a sub-nanopore at an applied potential of 0.7 V. The blue dotted line box highlights a portion of the data in which the ACFs were calculated. The cartoon shows the assumed molecular configuration with the arrow indicating the direction of the cantilever motion. (2b) A magnified view showing the change in force (top) and blockade current (bottom) of the highlighted data. (2c,2d; left) The corresponding ACFs of the force and current, respectively of the traces in (2b). (2c, 2d; right) Kymographs of the force and current, respectively representing a compilation of ACFs similar to (2c, 2d; left) obtained with a 2 nm window, but with a start staggered by 0.01 nm. (2e) Protein sequence analysis showing a single trace (blue) juxtaposed with an occluded volume model (assuming k=4, red). The error map above the plot indicates the read fidelity. Positions where the read departs from the model less than 20% are represented in gray as correct reads. (2f) Fractional mean read error as a function of volume of a residue in H3.2 as indicated. Smaller AAs are a dominant source of error. (2g) Read error for each AA residue in H3.2. By assigning positional errors to the a-priori sequence associated a read position, the absolute mean read error for each AA residue was calculated. The errors were converted to volume differences via the model for the AA residues constituting H3.2. (2h) The Hooge coefficient, derived from the pink noise power spectral density in the range 1-50 Hz, normalized by the square of the open pore current, plotted as a function of the open pore current squared for a sub-nanopore pore with a 0.6×0.7 nm2 cross-section. Inset: The power spectral density (PSD) for the same pore with open pore current as a parameter. The low frequency portion of the spectra was dominated by l/f noise as indicated by the dashed lines.

FIG. 3a-3e. Correlated current through sub-nanopores. (3a) The Hooge coefficient, derived from the pink noise power spectral density in the range 1-100 Hz, normalized by the square of the open pore current, plotted as a function of the open pore current squared for a collection of sub-nanopores ranging from 0.3 nm to 1.0 nm in diameter. Observations for S_{l/f}/I²>10⁻³ Hz⁻¹ are generally consistent with a noise resulting from uncorrelated mobility fluctuations in the electrolyte in the pore until a jamming threshold current is reached. Beyond the jamming threshold (indicated by the dotted vertical lines), the noise power spectral density (PSD) is independent of the current indicating correlations between fluctuations. (3b) The dependence of the jamming threshold current, Iₜ, on the pore cross-sectional area estimated from TEM. (3c) That portion of a current blockade associated with a force plateau analyzed into sixty segments (ten of which are shown) according to the position of the minima in the force to identify a quadromer trap in the pore. (3d) The rms-variation in the blockade level associated with all of the segments in (3c) plotted as a function of the fractional blockade indicating the noise is suppressed at higher fractions. (3e) The frequency content, measured by the ratio of the low to high frequency signal power, as a function of the fractional blockade, showing that nonlinearity in the signal becomes more pronounced as the fractional blockade improves.

FIG. 4(a)-4(b). 4(a) The force (top) and current (bottom) measured as H3.2 is peeled from the surface of a silicon nitride membrane at 19.66±0.06 nm/s against an applied potential of 0.4 V. The molecule is not in a nanopore, as evident by the lack of a current blockade. The green box highlights a 4 nm portion of the data in which the ACF is calculated. (4b) The corresponding ACF of the force (top) and the current (bottom) obtained from the windows in (4a). No regular features are observed when the molecule is peeled from the membrane surface.

FIG. 5 (a)-5c. Detecting single proteins using a sub-nanopore. (5a-c, i) TEM micrographs of pores with nominal diameters of 0.7 nm; 0.5 nm and 0.3 nm respectively, sputtered through silicon nitride membranes that are about 10 nm thick. The shot noise is associated with electron transmission through the pore. (5a-c, ii) Multislice simulations of the TEM images consistent with the experimental conditions. The simulation (5a,ii) corresponds to a bi-conical pore with a 0.8×0.7 nm² cross-section and a 20° cone angle at defocus of −40 nm. (5b,ii) corresponds to a bi-conical pore with a 0.5×0.7 nm² cross-section and a 20° cone angle at defocus of −40 nm. (5c,ii) corresponds to a bi-conical pore with a 0.4×0.5 nm² cross-section and a 20° cone angle at defocus of −40 nm. The close correspondence between the simulations and the actual TEM images proves that the models accurate reflect the actual pore structure. (5a-c, iii) Two-dimensional projections from the top through the model showing the atomic distribution near the pore waist. The atoms are represented by a space-filling model in which Si is a sphere with a 0.235 nm diameter and N is a sphere with a 0.13 nm diameter. (5a-c, iv) 3D, perspective of space-filled representations of the pore models. For clarity, only atoms on the pore surface are shown. (5d) Consecutive current traces illustrating the distribution of the duration and fractions of blockade currents associated with translocations of single molecules of CCL5 through a 0.5×0.6 nm² pore at 1V. In the figure, higher values correspond to larger blockade currents. (5e) Schematic of the translocation of a protein through a sub-nanopore. Denaturing agents impart a uniform negative charge to the protein, rendering it into a rod-like structure. (5f-h) Heat maps that characterize the distribution of fractional blockade relative to the open pore current (ΔI/I₀) versus the duration (Δt), associated with denatured CCL5 translocating through pores with a 1.4×1.6 nm², 0.5×0.6 nm2 and 0.3×0.3 nm² cross-section respectively at 1V. The white contour indicates a region containing 50% of the events. The same CCL5 contour in (5f) is represented in (5g) and (5h) in gray to illustrate that the median fraction blockade increases as the pore cross-section was diminished. (i) Like (f), heat maps showing the blockade current distribution associated with denatured BSA translocating through the same 1.4×1.6 nm² pore at 1 V. This distribution was easily distinguished from CCL5 since the energy distance was large, i.e. Δ=1.6×10-⁴. (5j,k) Heat maps that characterize the distribution of blockades associated with denatured H3N and H3A, respectively, translocating through pore with a 0.5×0.6 nm² cross-section at 0.7 V. These distributions could not be easily distinguished since the energy distance was only A=3.8×10⁻⁵.

FIG. 6(a)-6(d). Detecting amino acids in a single protein using a sub-nanopore. (6a) An expanded view of a single blockade (from FIG. 5b) illustrating nearly regular fluctuations in the blockade current associated with a single CCL5 molecule translocating through a pore with a 0.5×0.6 nm² cross-section, which were attributed to individual residues translocating in a turn-style fashion. The gray trace represents unfiltered, unfitted raw data whereas the black line is the smoothed data. The orange circles identify the peaks in the trace. (6b-d) Like (6a), an expanded view illustrating nearly regular fluctuations, the number of which is associated with the number of AAs in mature CXCL1 through 0.5×0.6 nm², BSA through 0.5×0.6 nm², and H3N through 0.5×0.6 nm², respectively. (6) The number of fluctuations tallied from blockades with different duration for the same four proteins. The tallies were independent of the blockade duration, but were dependent on the number of residues in the protein.

FIG. 7(a)-7(g). Protein sequence analysis. (7a, top) A 400 blockade consensus for CCL5 through a pore with a 0.5×0.6 nm² cross-section, (black) juxtapossed with an occluded volume model (assuming k=3, red), and a single highly correlated event (A=0.69, blue). The error map above the plots indicates the read fidelity. Positions where the read departs from the model less than 20% are represented in gray as correct reads. (7a, bottom) A gray-scale error map illustrating correct reads and misreads. The AA positions where the read departs from the model less than 20% of the time are represented in gray, whereas black reflects a misread. (7b, top) Like (a, top), but showing a 45-blockade consensus (black) for CXCL1 through a different pore with a similar 0.5×0.6 nm² cross-section, juxtapossed with an occluded volume model (assuming k=5, red) and a single (blue) event (A=0.67). (7b, bottom) Like (a), but for CXCL1. Despite the lower number of blockades, the read fidelity improves for CXCL1 as evident from the error map. (7c, top) Like (a, top), but showing a 52-blockade consensus for H3N through a pore with a 0.5×0.6 nm² cross-section juxtaposed with an occluded volume model (assuming k=5, red) and a single (blue) event (A=0.68). (7c, bottom) Like (a, bottom), but showing a gray-scale error map illustrating correct and incorrect reads (gray/black) for H3N. (7d-f) By assigning positional errors to the a-priori sequence associated a read position, the absolute mean read error at each AA residue can be calculated. The errors were converted to volume differences via the model for the AA residues constituting CCL5, CXCL1 and H3N, respectively. (7g) Fractional mean read error as a function of volume of a residue in CXCL1, CCL5 and H3N as indicated. Smaller AAs are a dominant source of error. The dotted lines (dark blue, light blue and red) are least-square fits to the data and provide a guide to the eye.

FIG. 8(a)-8(b). Detecting single-site modifications to a histone H3 tail peptide using a sub-nanopore. (8a,b, top) To explore the effect of single-site chemical modifications on the blockade fluctuation amplitude, native H3 (light blue, 304 events) and K9-acetylated H3A (dark blue in (8a), 231-blockades, scaled to native H3) and K9-methylated H3M (dark blue in (8b), 958-blockades, scaled to native H3) consensuses were formed, juxtaposed on the same plots and compared. The fluctuation amplitudes were enhanced between positions 6 and 11 indicating an increased occluded volume there. (8a,b, bottom) The difference between the native and modified consensus traces (gray) showed a broad top-hat-like increase in fractional blockade (dotted black line) associated with the acetylation/tri-methylation site. When fitted, the consensus difference indicated that a single site modification resulted in changes in occluded volume ranging over 3.9 AA residues (18.5% of the trace) for single site acetylation and 4.2 AA residues for single site methylation. Although, single-site resolution was not indicated, single-site modifications can be clearly observed. Furthermore, near the center of the consensus difference, a prominent fluctuation peak was also evident at position 8.9, which was tentatively attributed to acetylation at position K9. Likewise, a weaker fluctuation was evident at position 9.0 to methylation at K9.

FIG. 9. Schematic of the IgG antibody structure. Heavy-chains (blue and light blue); light-chains (green and light green). The heavy-chains consist of three constant domains (CH1, CH2, CH3), one variable domain (VH). The light-chains consist of a constant domain (CL) and a variable (VL). Fc is the part that binds to the cell surface; while the antigen attaches to the antibody binding sites located at the ends of the Fab arms. There are three CDR loops per variable domain (L1, L2, L3 on the light-chain and H1, H2, H3 on the heavy.) The CDRs are actually part of the domains VL and VH. Adapted from Noviimune.com.

FIG. 10. Detecting proteins with a sub-nanopore. (10 a,i) TEM micrograph of a pore with nominal 0.5 nm diameter, sputtered through silicon nitride membrane about 10 nm thick. The shot noise is associated with electron transmission through the pore. (10a,ii) Multislice simulations of the TEM images consistent with the experimental conditions. The simulation corresponds to a bi-conical pore with a 0.4×0.5 nm2 cross-section and a 200 cone angle at defocus of −40 nm. The close correspondence between the simulations and the actual TEM images proves that the model accurately reflects the actual pore structure. (10a,iii) Projection from the top through the model showing the atomic distribution near the pore waist. The atoms are represented by a space-filling model (10a,iv) 3D, perspective of space-filled representation of the pore model. For clarity, only atoms on the pore surface are shown. (10b) FES simulation of the electric potential along the vertical z-axis for a 0.5-nm-diameter pore with a 150 cone angle through an 8-nm-thick silicon nitride membrane in 1 M NaCl at 1 V bias. Inset: Simulation of electric field (V/m) distribution. (10c) The electric field along the z-axis for the pore shown in (b). (d) Juxtaposition of the measured current-voltage characteristic and simulations for different pores. (e) 3 nm Consecutive current traces illustrating the distribution of the duration and fractions of blockade currents associated with translocations of single molecules of CCL5 through a 0.5×0.6 nm2 pore at 1V. In the figure, higher values correspond to larger blockade currents. (f) Schematic of the translocation of a protein through a sub-nanopore. (g-i) Heat maps that characterize the distribution of fractional blockades relative to the open pore current (ΔI/I0) versus the duration (Δt), associated with denatured CCL5 translocating through pores with a 1.4×1.6 nm2, 0.5×0.6 nm2 and 0.3×0.3 nm² cross-section respectively at 1V. The white contour indicates a region containing 50% of the events. The same contour in (g) is represented in (h) and (i) in gray.

FIG. 11 (a)-11 (c). Detecting amino acids in a single protein using a sub-nanopore. (a) An expanded view of a single blockade (from FIG. 9e) illustrating nearly regular fluctuations in the blockade current associated with a single CCL5 molecule translocating through a pore with a 0.5×0.6 nm2 cross-section, which were attributed to individual residues translocating in a turn-style fashion. The gray trace represents unfiltered, unfitted raw data whereas the black line is the smoothed data. The orange circles identify the peaks in the trace. (11b) Like (11a), an expanded view illustrating nearly regular fluctuations, the number of which is associated with the number of AAs in mature H3N. (11c) The number of fluctuations tallied from blockades with different duration for the same four proteins. The tallies were independent of the blockade duration, but were dependent on the number of residues in the protein.

FIG. 12 (a)-12 (d). The translocation kinetics of a single protein molecule through a sub-nanopore measured with AFM. (12a) Schematic representation of the apparatus used to measure the force and current associated with the translocation of a single protein in a sub-nanopore. The sub-nanopore in a Si₃N₄ membrane is embedded in a two-layer (cis/trans) microfluidic device made from PDMS. A voltage is applied between a Ag/AgCl electrode embedded in the trans-channel and the current is measured using an amplifier. (12b) Cutaway of the schematic showing biotinylated H3.2 tethered to the tip of an AFM cantilever through a bond to streptavidin, translocating through the pore. (12c) Concomitant direct measurements of the force and current as H3.2 was impelled through a pore with a 1.0×1.3 nm² cross-section. The force (top) on protein and the ionic blockade current (bottom) under an applied potential of 0.7 V measured while the AFM cantilever was retracted from the pore at 4.0 nm/s showing a relatively frictionless plateau in the force. Inset: The cartoon shows the assumed molecular configuration with the arrow indicating the direction of motion. (12d) Like (c), the force (top) and current blockade (bottom) observed under the same conditions, but instead showing a single H3.2 stretching in the pore, producing a force-extension curve reflecting the molecular elasticity. The blue line represents a fit to the FJC model for the stretch.

FIG. 13(a)-13 (b). 13(a) Optical micrograph of a Ag/AgCl annulus encircling a thin nitride membrane; 13 (b) An AFM topograph of the same annulus.

FIG. 14 (a)-14 (e). Forces and currents associated with a single H3.2 molecule sliding frictionlessly through a 0.6×0.7 nm2 sub-nanopore. (a) The force (top) and the blockade current (bottom) measured as H3.2 is extracted at 4.0 nm/s from the pore against 0.7 V. The blue dotted box highlights a portion of the data in which the ACFs were calculated. The cartoon shows the assumed molecular configuration with the arrow indicating the direction of the cantilever motion. (b) A magnified view showing the change in force (top) and blockade (bottom) of the highlighted data. (c,d; left) The ACFs of the force and current, respectively of the traces in (b). (c,d; right) Kymographs of the force and current, respectively representing a compilation of ACFs similar to (c,d; left) obtained with a 2 nm window, but with a start staggered by 0.01 nm. (e) Protein sequence analysis showing a single trace (blue) juxtaposed with an occluded volume model (assuming k=4, red). The error map above the plot indicates the read fidelity. A correct (incorrect) read is represented in gray (black).

FIG. 15 (a)-15 (c). Protein sequence analysis. (a, top) A 45 blockade consensus (black) for CXCL1 through a pore with a 0.5×0.6 nm2 cross-section, juxtaposed with an occluded volume model (assuming k=5, red), and a single highly correlated event (C=0.67, blue). The error map above the plots indicates the read fidelity. (a, bottom) A gray-scale error map illustrating correct reads and misreads. (b, top) Like (a, top), but showing a 52-blockade consensus for H3N through a pore with a 0.5×0.6 nm2 cross-section juxtaposed with an occluded volume model (assuming k=3, red) and a single (blue) event (C=0.68). (b, bottom) Like (a, bottom), but showing a gray-scale error map illustrating correct and incorrect reads (gray/black) for H3N. (c, top) Like (a, top), but showing a 190-blockade consensus (C=68) for IgG4 through a pore with a 0.5×0.6 nm² cross-section juxtaposed with an occluded volume model (assuming k=4, red) and a single (blue) event (A=0.48). (c, bottom) Like (a, bottom), but for IgG4.

FIG. 16. Detecting PTMs in a histone H3 tail peptide using a sub-nanopore. (top) To explore the effect of single-site chemical modification on the blockade fluctuation amplitude, native H3 (light blue, 304 events) and K9-acetylated H3A (dark blue, 231-blockades, scaled to native H3), consensuses were formed, juxtaposed on the same plot. The fluctuation amplitudes were enhanced between positions 6 and 11 indicating an increased occluded volume there. (bottom) The difference between the native and modified consensus traces (gray) showed a broad top-hat-like increase in fractional blockade (dotted black line) associated with the acetylation site. When fitted, the consensus difference indicated that a single site modification resulted in changes in occluded volume ranging over 3.9 AA residues for single site acetylation. Furthermore, near the center of the consensus difference, a prominent peak was also evident at position 8.9, which was tentatively attributed to K9 acetylation.

FIG. 17—A PCA decomposition of training data shows clusters of k-mers having the same signal value (encoded with color).

FIG. 18 (a)-18 (e)—Detecting proteins with a sub-nanopore. (a,i) TEM micrograph is shown of a pore with nominal 0.5-nm diameter, sputtered through a silicon nitride membrane ˜10 nm thick. The shot noise is associated with electron transmission through the pore. (a,ii) Multi-slice simulations of the TEM images are shown, consistent with the imaging conditions. The simulation corresponds to a bi-conical pore with a 0.4×0.5 nm² cross-section and a 20° cone angle at defocus of −40 nm. (a,iii) Projection from the top through the model showing the atomic distribution near the pore waist. The atoms are represented by a space-filling model. (a,iv) 3D, perspective of space-filled representation of the pore model. For clarity, only atoms on the pore surface are shown. (b) FES simulation is shown of the electric potential along the vertical z-axis for a 0.5-nm-diameter pore with a 15° cone angle through an 8-nm-thick silicon nitride membrane in 1 M NaCl at 1 V bias. Inset: Simulation of electric field (V/m) distribution. (c) The electric field is shown along the z-axis for the pore shown in (b). (d) Juxtaposition of the measured current-voltage characteristic and simulations for different pores. (e) Consecutive current traces are shown that illustrate the duration and fractional blockade current associated with translocations of CCL5 molecules through a 0.5×0.6 nm² pore. Higher values correspond to larger blockade currents. (f) Schematic of the translocation of a protein through a sub-nanopore.

FIGS. 19 (a) to 19 (c). Detecting AAs in a single protein using a sub-nanopore. (a) An expanded view of a single blockade (from FIG. 1e) illustrating nearly regular fluctuations in the blockade current associated with a single CCL5 molecule translocating through a pore with a 0.5×0.6 nm² cross-section, which were attributed to individual AAs translocating in a turn-style fashion. The gray trace represents unfiltered, unfitted raw data whereas the black line is the smoothed data. The orange circles identify the peaks in the trace. (b) Like (a), an expanded view illustrating nearly regular fluctuations, the number of which is associated with the number of AAs in H3N (c) The number of fluctuations tallied from blockades with different durations for the same proteins. The tallies were independent of the blockade duration, but were dependent on the number of residues in the protein.

FIG. 20(a)-FIG. 20 (e). Protein sequence analysis. (a, top) A 45-blockade consensus (black) for CXCL1 through a pore with a 0.5×0.6 nm² cross-section, juxtaposed with an AA volume model (assuming k=5, red), and a single highly correlated event (C=0.67, blue). The error map above the plots indicates the read fidelity. (a, bottom) A gray-scale error map illustrating correct reads and misreads. (b, top) Like (a, top), but showing a 52-blockade consensus for H3N through the same pore cross-section, juxtaposed with an AA volume model (assuming k=3, red) and a single (blue) event (C=0.68). (b, bottom) Like (a, bottom), but showing an error map for H3N. (c, top) A 58-blockade consensus for the block co-polymer R-G through a sub-nanopore with a 0.4×0.5 nm² cross-section (red) is shown juxtaposed with an occluded volume model (assuming k=4, black) and single highly correlated blockades (C=0.90 and 0.96, blue). (c, bottom) The magnitude of the consensus of fluctuations obtained from the difference between individual blockades and the volume model is shown. (d,e, top) The effects of single-site chemical modifications to the histone H3 tail peptide on the blockade fluctuation amplitude are illustrated. Native H3N (light blue, 304 events) and K9-acetylated H3A (dark blue in (c), 231-blockades, scaled to native H3) and K9-methylated H3M (dark blue in (d), 958-blockades, scaled to native H3N) consensuses were formed, juxtaposed on the same plots and then compared. It was observed that the fluctuation amplitudes were enhanced between positions 6 and 11 indicating an increased occluded volume there. (c,d, bottom) These figures relate the differences between the native and modified consensus traces (gray). The differences showed a broad top-hat-like increase in fractional blockade (dotted black line) associated with the acetylation/tri-methylation site. When fitted, the consensus difference indicated that a single site modification resulted in changes in occluded volume ranging over 3.9 AA residues (18.5% of the trace) for single site acetylation and 4.2 AA residues for single site methylation. Although, single-site resolution was not indicated, single-site modifications can be clearly observed. Furthermore, near the center of the consensus difference, a prominent fluctuation was also evident at position 8.9, which was tentatively attributed to acetylation at position K9. Likewise, a weaker fluctuation was evident at position 9.0 to methylation at K9.

FIG. 21 (a)-FIG. 21 (e).—Detecting single protein molecules using a sub-nanopore. (a) A schematic representation of the apparatus used to measure the force and current associated with a single protein translocating through a sub-nanopore is shown. The sub-nanopore in a silicon nitride membrane was embedded in a two-layer (cis/trans) microfluidic device made from PDMS. An electrical bias of +0.7 V was applied between Ag/AgCl electrodes embedded in the trans- and cis-channels respectively, and the current between them was measured using an amplifier. (b) Cutaway of the schematic showing a biotinylated H3 histone, tethered to the tip of an AFM cantilever through a bond to streptavidin (STR), translocating through the pore. (c-e) Direct measurements of the concomitant force and current as an H3.3 protein was impelled through a 0.5-nm-diameter with 0.01% (w/v) SDS on the trans-side of the membrane (c,d) and without it (e). The force (top) on the protein and the blockade current (bottom) were measured with an applied potential of +0.7 V, while the AFM cantilever was retracted from the pore at 4.00 nm/s, showing both slip-stick and a relatively frictionless plateau in the force. The dashed (blue) lines represent a fit to the FJC model for the stretches. On the other hand, the force plateaus (d,e) reflected nearly frictionless translocations. The dotted (cyan) lines offer guides to the eye. Insets: The cartoons show the assumed molecular configuration with the arrow indicating the direction of the cantilever motion. Inset to (e): Box-and-whisker plots are shown that summarize the distribution of force and current blockade measurements acquired from H3.2 and H3.3 done with SDS on the trans-side of the membrane, and without SDS on the trans-side, alongside the control, K₁₀₀.

FIG. 22 (a)-FIG. 22 The forces and currents measured as a single H3.3 histone was impelled through a sub-nanopore. (a) The figure shows the force (top), and the blockade current (bottom) measured as H3.3 was extracted at 4.0 nm/s from a 0.5-nm-diameter sub-nanopore against a potential of +0.70 V. The dotted (light blue) boxes highlight a portion of the data in which the ACFs were calculated. The cartoon shows the assumed molecular configuration with the arrow indicating the direction of the cantilever motion. The dotted (cyan) lines offer guides to the eye. (b) A magnified view of the highlighted region in (a) is shown that illustrates fluctuating patterns in the force and current after subtracting the mean (□). The circles denote the fluctuations above the noise identified using a 2□-criterion. The (blue) vertical lines are used to facilitate the comparison of the alignment of the force fluctuations relative to the current fluctuations. Inset: A plot is shown that depicts the cross-correlation between the positions of the force and current fluctuations. The average positional difference between the force and current peaks was r=0.20 nm with a p-value of 0.0004 when compared to a random sequential peak placement. (c; left) The corresponding ACFs of the force (top) and current (bottom) are shown of the traces highlighted by the dotted (cyan) lines in (c; right). (c; right) Kymographs of the force (top) and current (bottom) are shown representing compilations of ACFs similar to (c; left) obtained with a 3 nm window with a start staggered by 0.1 nm. (d) The figure compares a single extraction with a consensus (compilation) of twelve similar extractions to illustrate the reproducibility and signal-to-noise.

FIG. 23 (a)-FIG. 23 (c) Protein sequence analysis. (a; top) A quadromer error map is shown that indicates the read fidelity for a single molecule of the H3.3 variant. The positions where the empirical reads shown in (a, bottom) are correct/incorrect are represented in gray/black, whereas no read at all is represented in orange. (a; bottom) The figure juxtaposes a compilation of the blockade currents associated with the frictionless retraction of a single H3.3 (blue) from a 0.5-nm-diameter sub-nanopore at a +0.70 V bias, with a volume model (pink). The AA volume models for the H3.3 variant assumes that k=4. (b) Like (a), the figure shows (top) a quadromer error map and (bottom) a juxtaposition of the blockade current, but associated with a single H3.2 (light blue) acquired under the same conditions from the same sub-nanopore with a volume model (red). (c; top-left) A heat map is shown that conveys twelve typical signed differences between single molecules of H3.2 and H3.3. A salient feature is repeatedly observed near read position 90. (c; bottom) The magnitude of the difference between the compilations acquired from the H3.2 and H3.3 variants in (a,b) is shown for a compilation of six blockades (black) and a single blockade (gray) along with the difference between the corresponding volume models for the same molecules (red). The H3.2 trace overlaps a practically identical H3.3 trace except near the read positions 32, 88, 90, and 91, where AA substitutions occur. Insets: Histograms are shown that relate the frequency of the difference measured for the compilation (single blockade) in (c), indicating that the peak observed near read position 90 is 4.5 σ (3.6σ) above the baseline.

FIG. 24 (a)-FIG. 24 (e).—Noise in a sub-nanopore (a-c, left) Current traces of blockades, □I, are shown, which were acquired in 250 mM NaCl at pH 7/pH 7/pH 3.3 with 0.7 V applied, associated with a single H3.3/K₁₀₀/K₁₀₀ molecule tethered to an AFM cantilever, translocating frictionlessly through a sub-nanopore with 0.6×0.7 nm² cross-section, respectively. The fluctuations in the current were diminished during the K₁₀₀ pH 7 blockade. (a-c, right) The corresponding PSDs are shown with (blue) and without (red) a blockade. (d) The l/f (pink) component of the noise PSD over the range 0.01 Hz to 100 Hz (Sₗ), normalized by the square of the open pore current _{I}_{₀}_{²}, measured in 250 mM NaCl electrolyte at pH 7, is plotted as a function of _{I}_{₀}_{²}, for a range of sub-nanopore diameters. The normalized noise power for I₀<1 pA was generally consistent with noise resulting from uncorrelated current fluctuations until a threshold current, Iₜ (delineated by the vertical grey dotted lines). The (black) dotted lines offer a linear extrapolation of _{S}_{ₗ}_{/I}_{₀}_{²} with _{I}_{₀}_{²}. Beyond the threshold, _{S}_{ₗ}_{/I}_{₀}_{²} is independent of the current indicating correlations between fluctuations. Inset: The dependence of the threshold current, Iₜ, on the pore cross-sectional area. The best-fit line extrapolates at zero threshold to cross-sections smaller than the respective hydrated ions, indicating that transport through a sub-nanopore forces dehydration. (e) Like (d), but comparing data acquired from an open 0.6×0.7 nm² cross-section pore in 250 mM NaCl at pH 7, blockaded with K₁₀₀ at pH 7 (black box) and at pH 3.3 (black dot).

FIG. 25 (a)-FIG. 25 (d)—Machine learning for discriminating protein. (a,b) A comparison is shown between the naïve volume (left) and RF regression model (right) for two proteins: H3N (top) and CCL5 (bottom), for a consensus of ten blockades. The RF-model shows an improved fit to the data as indicated by the PCC. (c) Signed error for AAs constituting H3.2 protein in order of increasing volume. The volume model (top) tends to underestimate signals associated with small volumes whereas the RF-model (bottom) shows no bias. (d) The median p-value is shown as a function of the number of blockades in a cluster for H4 and H3.3 trained on H3.2. The solid lines represent exponential fits. The decoy database size is 10⁵ for H4 and 5×10⁶ for H3.3. The p-value vanishes for a consensus >10.

### DETAILED DESCRIPTION OF THE PREFERRED EMBODIMENT

Throughout the specification and claims, the following terms take the meanings explicitly associated herein, unless the context clearly dictates otherwise.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the term “or” is an inclusive “or” operator and is equivalent to the term “and/or” unless the context clearly dictates otherwise.

The term “based on” is not exclusive and allows for being based on additional factors not described, unless the context clearly dictates otherwise.

The term “a,” “an,” and “the” include plural references. Thus, “a” or “an” or “the” can mean one or more than one. For example, “a” cell and/or extracellular vesicle can mean one cell and/or extracellular vesicle or a plurality of cells and/or extracellular vesicles.

The meaning of the term “quadromer” is a short length of four amino acids.

The meaning of the abbreviation “AA” is amino acid. An amino acid is the smallest unit of protein and is an organic molecule made up of amine and carboxylic acid functional groups. An amino acid is composed of nitrogen, carbon, oxygen, and hydrogen molecules.

TABLE 1
List of amino acids (AA), their
symbols and abbreviations.

| Amino acid | Abbreviation | Symbol |
|---|---|---|
| Phenylalanine | Phe | F |
| Leucine | Leu | L |
| Serine | Ser | S |
| Tyrosine | Tyr | Y |
| Cysteine | Cys | C |
| Tryptophan | Trp | W |
| Leucine | Leu | L |
| Proline | Pro | P |
| Histidine | His | H |
| Glutamine | Gln | Q |
| Arginine | Arg | R |
| Isoleucine | Ile | I |
| Methionine | Met | M |
| Threonine | Thr | T |
| Asparagine | Asn | N |
| Lysine | Lys | K |
| Serine | Ser | S |
| Arginine | Arg | R |
| Valine | Val | V |
| Alanine | Ala | A |
| Aspartate | Asp | D |
| Glutamate | Glu | E |
| Glycine | Gly | G |

The meaning of “in” includes “in” and “on.”

The meaning of the term “picometer” (symbol “pm”) is as a unit of length in the metric system equal to one millionth of a micrometer (also known as a micron), and used to be called a micromicron, stigma or bicorn. One picometer is 1/1,000 of a nanometer. The symbol “μμ” was once used for picometer. The symbol “pm” is used in the present disclosure to mean picometer.

The meaning of the term “sub-micrometer” as used in the description of the present invention means a unit length that is less than a micrometer. A sub-micrometer size pore, or a “sub-micropore”, is intended to include a pore having a size that is less than 1,000 nanometers (nm) in diameter, or less than 1,000,000 picometers (pm) in diameter. The symbol “μm” will be used to denote micrometer in the present description.

The meaning of the term “sub-nanometer” as used in the description of the present invention means a unit of length that is less than a nanometer. A sub-nanometer size pore is a pore having a diameter that is less than a nanometer (nm) in diameter. A nanometer is defined as a unit of length in the metric system of one billionth of a meter (0.000000001 m). One nanometer equals 1,000 picometers, and one nanometer equals ten angstroms. Nanometer is often denoted by the symbol mμ, or sometimes more rarely as μμ. For purposes of the present description, nanometer will be “nm”. A sub-nanometer size pore can be a pore having a picometer range of diameter size, such as less than 1,000 picometers, or less than 900 pm (0.9 nm), or less than 800 pm (0.8 nm), or about 300 pm to less than 1,000 pm, or about 300 pm (0.3 nm) to about 900 pm (0.9 nm).

The meaning of the term “sub-nanopore” as used in the description of the present invention is a pore having a diameter that is smaller than a nanometer (nm). One nanometer is equal to 1,000 picometers (pm).

The following examples are provided to demonstrate and further illustrate certain preferred embodiments and aspects of the present technology, and they are not to be construed as limiting the scope of the technology

### Example 1—Correlations in the Forces, Currents and Noise Characterizing the Translocation of a Single Protein Molecule Through a Sub-Nanopore

In the present example, the force and blockade current characterizing the translocation of a single protein molecule, tethered to the tip of an atomic force microscope (AFM) cantilever, were measured as the molecule was impelled systematically through a thin inorganic membrane having pores of a sub-nanometer size (“sub-nanopore” size pores). The force measurements revealed a dichotomy in the translocation kinetics: either the molecule slid nearly frictionlessly through the pore or it slipped-and-stuck. When the molecule translocated frictionlessly through a sub-nanopore, periodic fluctuations were observed in force and blockade current with lags that corresponded to the separation between AAs, and the amplitudes of the fluctuations in the blockade were correlated with the occluded volume of short lengths of amino acids, specifically four amino acid length sequences (“quadromers”), of the protein sequence.

It was observed that the cross-section of the sub-nanopore was comparable to the size of the hydrated ions. Correlations developed in the current that were evident in the noise even in the absence of a molecule in the pore, which seems to enhances the signal-to-noise ratio associated with the measurement of the occluded volumes of AAs.

Protein sequencing, like DNA sequencing, is indispensable in the analysis of biology.⁽¹⁾ However, unlike DNA, proteins are not as amendable to amplification.

Template-dependent replication and amplification are at the core of commercial DNA sequencing technologies. Even though it is error-prone (1 error/9000 nucleotides) and the amplicon size is restricted to <3,000 base-pairs, there is an overwhelming advantage conferred by amplification. It expands the coverage, which leads to high accuracy even when the read are low fidelity. Without amplification, the limitation on protein concentration is acutely felt in the methods used for sequencing protein such as mass spectrometry (MS), which require concentrations >fmole/L-scale.

The primary structure of a protein consists of a linear sequence of amino acids (AAs) linked by peptide bonds separated by about 0.38 nm in equilibrium. The average/median mass of a (human) protein is about 53 kDa/42 kDa, which corresponds to about 485/384 AA residues.⁽²⁾ The number of amino acid residues in an amino acid sequence poses a challenge. On the one hand, Edman degradation has low throughput with short (less than 30 amino acid residues long) peptide reads, requiring proteolytic digestion and peptide fractionalization. On the other hand, MS can sequence a peptide/protein of any size, but it relies on enzymatic digestion, and therefore becomes computationally demanding to reassemble the digested sequence as the size increases. The single molecule sequencing techniques proposed in the present disclosure by enabling the maximum amount of information to be extracted from a minimum of material, provides a previously unknown and powerful tool in the art of protein sequencing technology.

When a sub-nanopore was immersed in electrolyte and a voltage was applied across it, blockades in the pore current developed as denatured protein molecules were impelled through the pore. The blockades evinced regular fluctuations, the number of which coincided with the number of AA residues, with amplitudes that were highly correlated with the volume occluded by a quadromer (four AA residues) in the protein sequence.⁽³⁾ Thus, each fluctuation represented a read of a quadromer, although the read fidelity was low, comparable to the noise. To improve the fidelity, a consensus of reads acquired from >10 blockades was formed.

A single molecule, tethered to the tip of an atomic force microscope (AFM) cantilever is shown to be impelled systematically through a sub-nanopore. The force on the molecule and current through the pore during this event was measured. These measurements were used to inform on the translocation kinetics and to improve the signal-to-noise ratio (SNR) and the read fidelity. It was discovered that, exclusively in a sub-nanopore, the force and current fluctuate periodically with a lag that corresponded to the persistence length in the polymer or the separation between AA residues (˜0.4-5 nm). Moreover, the amplitude of the blockade current fluctuated in correspondence with the occluded volume of quadromers in the protein sequence. It was found that correlations in the current noise, that develop without a molecule in the pore, become exaggerated as the sub-nanometer cross-section of the pore shrinks. These correlations in the current were inferred from a (traffic) “jamming” transition in the noise associated with congestion that develops between the ions at high current, which supposedly gives rise to non-linear density waves and bunching of the carriers in the pore. Taken together, these data indicate that, as the occluded volume associated with each quadromer increased, the jamming threshold decreased, improving the SNR.

Two proteins were selected for sequencing with a sub-nanopore. One was a truncated human histone H3.2⁽⁵⁾ and a monoclonal antigen-binding fragment (Fab) of the antibody IgG4.⁽⁶⁾ H3.2 was chosen because of its importance in epigenetics—it is one of five histone proteins involved in the structure of chromatin in eukaryotic cells and post-translational modifications of it play a central role in the regulation of genes, and because it consists of a chain of 136 AA (15,388 Da, 15,565 Da), making it a nontrivial test of the technology. To measure the force and current associated with a translocation through a sub-nanopore, the biotinylated protein (formed by ligation via a native peptide bond to N-terminal biotin) was denatured by heat, sodium dodecyl sulfate (SDS) and β-mercaptoethanol (BME) to eliminate the higher-order structure and then the molecule was tethered to the tip of a soft (k=15-30 pN/nm) AFM cantilever using streptavidin (SA). The exact structure of the aggregate formed between SDS and the proteins remains unsolved.^{(9, 10)} In the present example, a “rod-like” model was adopted in which the SDS molecules form a shell along the length of the protein backbone.⁽⁹⁾

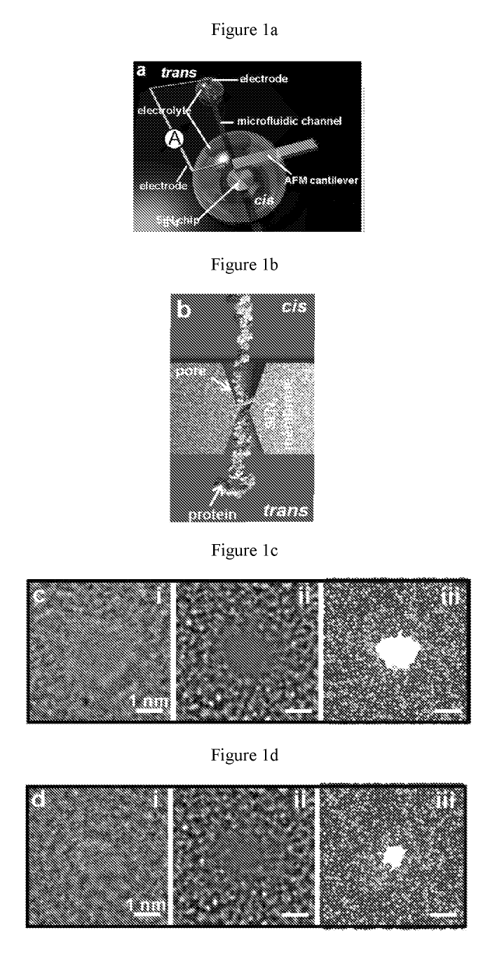

The force and the blockade current were measured simultaneously as the tethered protein was impelled by an AFM at a constant velocity through a sub-nanopore embedded in a microfluidic device immersed in 250 mM NaCl electrolyte (FIGS. 1a,b). The sub-nanopores were produced using a tightly focused electron beam in a Scanning Transmission Electron Microscope (STEM) to sputter through a thin inorganic silicon nitride membrane nominally 10 nm thick.⁽¹¹⁾ The topographies of the sub-nanopores were inferred from TEM. To accurately assess the topography, TEM micrographs were imitated by multislice simulations⁽¹²⁾. The close correspondence between the images (FIGS. 1c,i and 1d,i) and simulations (FIGS. 1c,ii, and 1d,ii), which reproduced the actual imaging conditions, indicated that the models (FIGS. 1c,iii, and 1d,iii) were realistic representations of the actual pores. The pores provided on the silicon nitride membrane were bi-conical, with cone-angles that ranged around θ=15±5°, and irregular, with cross-sections at the waist that were (1.0±0.1)×(1.3±0.1) nm² and (0.6±0.1)×(0.7±0.1) nm². As the mean distance between oxygen atoms in the water molecules in the first hydration shell surrounding a sodium ion is about 0.210 nm, the cross-section near the waist of the sub-nanopore was comparable to a hydrated ion.⁽¹³⁾

To locate the pore relative to fiducial marks, i.e. the edges of the (4×5 μm²) membrane, an AFM topographical scan was performed with a sharp (nominally 2 nm radius) unfunctionalized tip in air. After that, a second AFM cantilever functionalized with protein was clamped into the cantilever holder and then immersed in electrolyte. The pore location was reacquired in liquid through triangulation from the fiducial marks and quick, small area scan. Then a voltage bias was applied across the membrane and the pore current was measured continuously with an external amplifier, while the force on the cantilever was inferred from the deflection. The tip position above the membrane was determined by accounting for both the deflection and the z-height.

The force and current through the pore were recorded as the tip was advanced toward the membrane (toward the C-terminus) and retracted (toward the N-terminus) from it until the molecule vacated the pore. Infrequently, both the force and the current measurements reflected nearly simultaneously the capture and evacuation of the molecule by the pore. The electric field that develops when a voltage was applied facilitated the capture of the molecule in the pore as the tip was advanced toward it. Finite element simulations (FES) of pores with this topography revealed that the electric field was tightly focused in the pore, peaking at 2×10⁶ V/cm near the center of the membrane for this voltage bias and decaying to 6×10⁴ V/cm 10 nm above the opening. Moreover, the bi-conical topography crowds the ionic current at the waist into a region about 1.5 nm in extent.⁽³⁾ Naively, the fractional change in the blockade current can be related to the ratio of the occluded molecular volume to the pore volume: i.e. ΔVₘₒₗ/Vₚₒᵣₑ. For example, native H3.2 has 136 AA residues, so if the denatured protein has a rod-like shape, about 26 AAs would span the entire 10 nm thick membrane. However, if the effective thickness of the membrane is defined by the current crowding associated with the bi-conical topography, in the absence of a persistent native-like topology only four AAs would span the waist of the pore, and so the occluded volume for a 1 nm-diameter pore would be only about ΔVₘₒₗ/V^{eff}ₚₒᵣₑ=0.64 nm³/1.93 nm³=0.33, which is close to the empirical fractional blockade current, ΔI/I₀=40 pA/133 pA(=40/216 pA)˜0.33(0.19). In contrast, the occluded volume associated with a translocation through a 0.6 nm-diameter pore would be only about ΔVₘₒₗ/V^{eff}ₚₒᵣₑ=0.64 nm³/0.927 nm³=0.69.

A dichotomy was observed in the translocation kinetics: the protein either slid frictionlessly through the pore or it stuck-and-slipped (FIGS. 1e,f). For example, the force measured as the cantilever was retracted from the pore with a (0.8±0.1)×(1.3±0.1) nm² cross-section at a constant velocity of 4.00±0.02 nm/s against a potential of 0.85 V occasionally revealed a force plateau during the translocation of a single molecule (FIG. 1e). As illustrated by the typical example in the figure, as the tip was retracted from the pore, the adhesion between the tip and the silicon nitride membrane predominated for tip-surface separations <48 nm. However, when the tip was released from the surface at position (1), a force plateau was observed at 248±4 pN as a single H3.2 protein molecule, as indicated by the blockade current of ΔI=40.9±2.0 pA, translocated through the pore. Constant force plateaus like these were interpreted as the molecule sliding relatively “frictionlessly” through the pore. Plateaus in the force were also observed without the protein in the pore as the molecule was peeled from the surface of the membrane without any indication of a blockade in the current (Fig. S1). Such plateaus have also been reported when single-stranded DNA was impelled through a 1 nm diameter pore, but not as frequently.⁽¹⁴⁾

Using an AFM to measure force-extension curves, native protein tethered to an AFM cantilever has been unraveled to inform on protein folding and corresponding function, but a frictionless slide has not been observed before probably because prior work has not focused on denatured protein.⁽¹⁵⁻¹⁷⁾ While not intending to be limited by any theory, it seems likely that a denatured protein aggregate with SDS, especially if the structure was “rod-like” without any secondary structure, would produce a force plateau. On the force plateau, the forces may be associated with a combination of a relatively weak (W<F·Δl=2.5×10⁻²⁰ J≈3.6 kJ·mol⁻¹ AA⁻¹) hydrophobic adhesion between the aggregate structure and the silicon nitride surface, the electrophoretic force and the electroosmotic flow impelling the molecule through the pore. Importantly, neither the forces on the plateau or the blockade current fluctuate beyond the minimum noise as the molecule was pulled through the pore up to position (2), at which point the blockade was relieved returning to the open pore value and the force on the molecule vanished.

A second category of translocation kinetics was observed under similar conditions as the cantilever was retracted from another pore with a (0.8±0.1)×(1.0±0.1) nm² cross-section (FIG. 1f). After the tip was released from the surface at position (1), a single H3.2 was pulled with an initial force of 94.7±10.9 pN at a constant velocity of 4.00±0.02 nm/s against a potential of 0.7 V while occluding the pore, giving rise to a blockade ΔI=169±26 pA. The blockade current likely indicates an occluded volume larger than a denatured protein, which may be attributed to a persistent, native-like topology of the protein either clogging the pore or sticking to the membrane. (On the other hand, it could also indicate more than one molecule blockading the pore, although it seems unlikely due to the small pore volume, the observation of a single stretched bond and the coincidence between the force and blockade termination.) The protein was subsequently stretched by the differential force ΔF=37 pN over a distance of 11.5 nm until the bond ruptured at position (2), after which it slid relatively frictionlessly until it vacated the pore at position (3) and the current returned to the open pore value.

Usually, the loading of a single molecule like this produces a force-extension curve that reflects the elasticity of the molecule. The associated kinetics through the pore resembles a “slip-stick” motion in which the polymer rapidly slips as soon as the applied force exceeds the threshold for rupturing the adhesive bond between the aggregate and the membrane. Stretching events like these in unstructured protein can be described by statistical mechanical polymer elasticity models, of which the most commonly used are the freely jointed chain (FJC) model,⁽¹⁹⁾ the worm-like chain model.⁽²⁰⁾ Using the FJC, with a Kuhn length of b ˜0.31-0.56 nm, the effective spring constant associated with each stretching event was estimated from k_{eff}=3 k_{B}T/bX, where k_{B}T represents the thermal energy at 293° K and X is the extension of the protein relative to its total length to find k_{eff}=3.2±1.6 pN/nm, which is consistent with prior estimates.⁽¹⁹⁾ At a constant extraction velocity of 4.00 nm/s, this spring constant implies a loading rate of 13 pN/s, indicating a near-equilibrium loading regime and a single bond type.

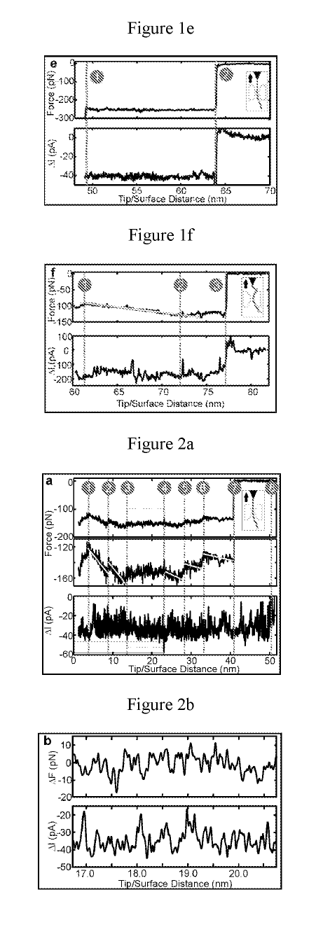

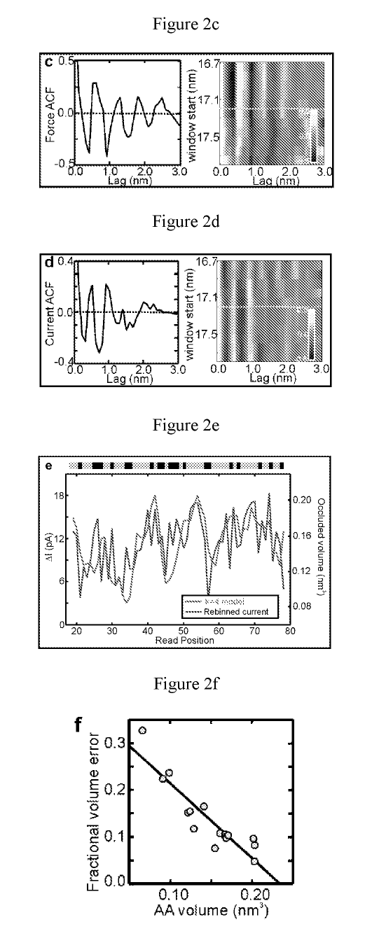

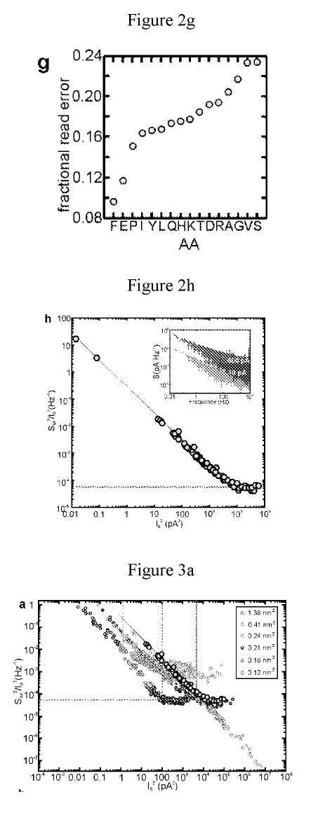

In a typical data set of 280 force curves, there were only 14 events in which the current was affected concurrently, of which 4 were considered frictionless, whereas the same data set contained 10 “slip-stick” force curves with concurrent blockades. In the subset of data for which the translocations were frictionless, scrutiny of the force and current fluctuations revealed regular correlated patterns intermittently (FIG. 2), but exclusively in sub-nanopores with cross-sections less than 0.6×0.7 nm². On the other hand, pores with larger cross-sections exhibiting force plateaus did not show evidence of correlations in the force or blockade current beyond the noise. The mean spacing between force/current fluctuations without a molecule in the pore (|dz|_{force}=0.20±0.06 nm) was consistent with white noise (|dz|ₙₒᵢₛₑ=0.24±0.13 nm). The rms current noise in a blockade was typically <12 pA-rms. Moreover, even after the baseline stretching force was subtracted, the residual forces and the corresponding current blockades associated with a single protein stretching in a pore did not exhibit regular, correlated fluctuations either like those observed on frictionless plateaus in a sub-nanopore.

Focusing exclusively on the subset associated with relatively frictionless kinetics through a sub-nanopore, FIG. 2 represents typical data acquired when a molecule was retracted (against 0.7 V at a constant velocity of 4.00±0.01 nm/s from a (0.6±0.1)×(0.7±0.1) nm². After the tip was released from the surface, the molecule sticks to the membrane at position (1), at a tip height of about 3.8 nm, corresponding to a blockade current of ΔI=33.5±6.4 pA, indicating that a single molecule was trapped in the pore. Starting at a force of 120.7±9.8 pN, as it translocates through the pore, the protein slip-and-sticks twice, stretching between positions (1) and (2) and then again between (2) and (3). Subsequently, the force is relatively constant until position (4) where it slips-sticks three times again. Finally, at position (7) the force on the molecule was relieved abruptly while the current remained blockaded before returning to the open pore value at position (8). Since the current returned to the open pore value after the force was relieved, it was inferred that the SA-biotin bond ruptured prematurely and the molecule remained for an additional ˜1.5 s before vacating the pore. Nevertheless, the 36.8 nm gap between the tip and the surface was comparable to (but less than) the contour length of the stretched molecule, which was estimated to be about 68 nm.

The molecule slides relatively frictionlessly through the pore in the gap between 15.5 and 23.5 nm. In that interval, periodicities were discernible in magnified views of the force and current (FIG. 2b), but they were even more apparent in the autocorrelation functions (ACFs) of the data (FIGS. 2c,d). The force exhibited intermittent regular oscillations (FIG. 2c; left) at a mean lag of 0.58±0.06 nm, while the current showed a mean lag of 0.47±0.07 (FIG. 2d; left), which corresponds closely with the distance between stretched residues. With 135±5 pN of tension applied, corresponding to the modulus of the denatured protein, the distance between residues was stretched from the equilibrium distance of 0.38 nm to about 0.5 nm. The separation between peaks in the force and current ACFs were nearly equidistant over short 10 nm range, as evident in the kymographs (FIGS. 2c,d; right) that represent a compilation of ACFs using a 3.0 nm moving window with a starting position that is staggered by 0.02 nm. The regularity of the fluctuations suggests a correlation between the orientation and topology of the residues in the pore. Interestingly, since the electric force is mainly confined to the 1.5 nm waist of the sub-nanopore, the correlations must extend over several (3-4) residues.

Taken altogether, these data indicate that the translocation of residue through a sub-nanopore on frictionless force plateau occurs in steps that are subject to control by the applied force. Since the fluctuations were correlated and nearly regular, it was inferred that the SDS must impart a nearly uniform negative charge density along the denatured protein. This would result in a consistent electric force working to impel the molecule through the pore.⁽³⁾ It is likely that counter-ions moved along with a protein molecule decorated by SDS to minimize Coulomb repulsion, but since a sub-nanopore has a diameter comparable to a hydrated ion, the motion of the ensemble through it would be impeded by steric hindrances. Thus, the regularity of the patterns were consistent with a tightly choreographed, turnstile motion of AAs through the pore in which a single AA stalls repeatedly in a well-defined conformation within the pore and then resumes when a sufficient force was applied to stretch or re-orientate the residue and impel it through.

Concomitantly, the volume occluded by the residues stalled in the pore waist must present a distinctive barrier to the flow of ions that is reflected as a fluctuation in the blockade current. Since both the force and current fluctuations were consistent with the separation between (stretched) residues, then each oscillation within a blockade current reflected an event in which one AA enters the pore while another leaves. Thus, it was reasoned that the fluctuation amplitude should be attributed to the occluded volume associated with the AA residues in the pore waist. Because the pore current was crowded and most of the potential dropped near the waist, it was further argued that the fluctuation amplitudes should measure the occluded volume within 1.5 nm of the waist.

These assertions were tested by correlating the blockade current to a model of the peptide chains in which each residue was represented by its volume estimate obtained from crystallography data.⁽²¹⁾ To account for the current crowding in the pore waist as the molecule steps successively, one AA at a time, through the peptide chain, a moving average with a window size ranging from k=4 AA volumes (depending on the molecular conformation in the pore) was performed on the sequence of volumes (FIG. 2e). The models based on AA volumes (Table 1) were found to be strongly correlated to the empirical data acquired from (0.6±0.1)×(0.7±0.1) nm² pore (C=0.52). The correlation degraded as k increased from four to ten indicating the likelihood that no more than four AA residues occupied the waist at one time, however.

The band above the plot in FIG. 2e represents the agreement for each read expressed as a percentage and subsequently identified as either a correct (gray) or incorrect (black) call, depending on whether the agreement was greater or less than 20%, respectively. The read accuracy from a single molecule was about 75%, more than 56 (standard deviations) above the reads acquired by fitting random noise. There are several qualifications required on the read accuracy. First, the number of correct reads obtained for each blockade does not reflect the accuracy with which single residues can be called. The threshold for a correct read was chosen to be 20%, which means that, on average, ±20% of optimally ranged and fitted random noise would fit the model because 40% of all data will fall within its threshold boundary. Nevertheless, the read accuracy from single molecules is statistically significant as per the null sets determined in prior work⁽³⁾ From this significance level, it was concluded that each fluctuation represents a low fidelity read that measures the occluded volume associated with quadromer in the waist of the pore.

Low fidelity reads with multiple monomers affecting the blockade should not pose a problem for sequencing protein since, in principle, a Viterbi algorithm⁽²²⁾ could be adapted to decode the sequence with single AA resolution as long as the translocation kinetics are stringently controlled. However, if a quadromer affects the blockade current, with the twenty common proteinogenic AAs, there are 20⁴ or 160,00 possible combinations to discriminate, which presents a formidable challenge for dynamic programming. Due to this multiplicity, systemic errors will disproportionally affect the read fidelity of a single molecule. By assigning errors to the a-priori sequence of cumulative AA volumes present at each position, the volume error for each AA was calculated (FIGS. 2f,g) that indicated the source of the errors. This analysis of the read fidelity exposed several interesting trends. Among them, it was observed that residues with small volumes (A, V, G, and S) were frequently misread, corroborating earlier work⁽³⁾ This makes sense since the volume associated with the error (0.020 nm³) is small relative to the effective pore volume, although it may be improved by increasing the cone angle or decreasing the pore diameter.

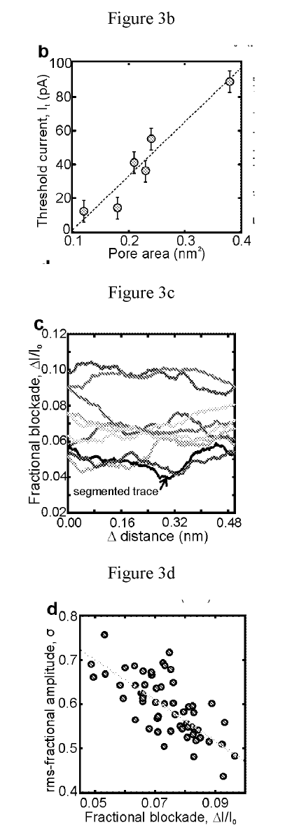

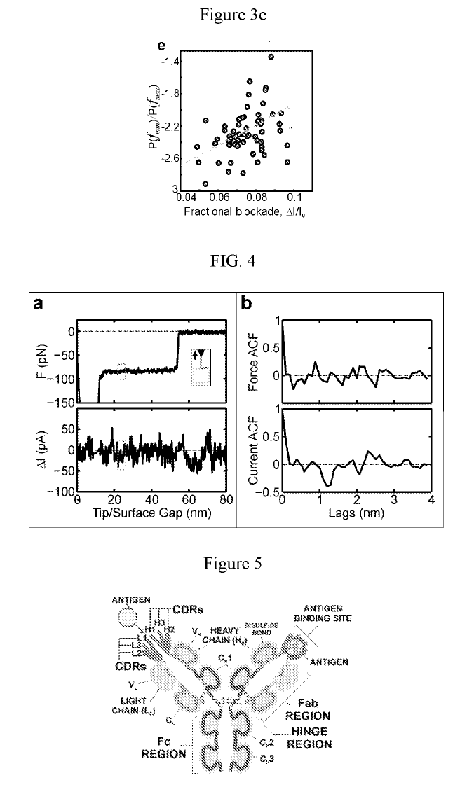

Regardless, considering the pore volume compared to the residues and the noise, the chemical specificity of a sub-nanopore was extraordinary. To account for the specificity, it was postulated that correlations develop in the ionic blockade current due to the occluded residue volume that enhance the signal. The correlations in the current were conspicuous in the noise (FIGS. 2h, 3a). Following Hooge, it has been shown that the pink (l/f) component (S_{l/f}) of the noise power spectral density (PSD) increases in proportion to the square of the current in pores filled with electrolyte >1 nm in diameter.^{(23, 24)} The corresponding fluctuations in the conductance, ΔG, have been attributed to variations in the electrolytic transport through the pore, i.e. S_{l/f}=I² A/f˜I²( [symbol] ΔG² [symbol] )/G²)/f=I²( [symbol] ΔI² [symbol] )/I²)/f where I is the current, G denotes the corresponding conductance and <ΔG²> is its mean square fluctuation (variance), A is the Hooge amplitude that depends on the type and concentration of charge carriers and f is the frequency.⁽²³⁾ Similarly, the low frequency PSD in sub-nanopores was also found to be inversely proportional to the frequency (FIG. 2h; inset) with an amplitude that depends on the inverse square of the current at low current (FIG. 2h). Moreover, the Hooge amplitude scaled with the pore resistance and inversely with the electrolyte concentration.

All of these trends follow from uncorrelated fluctuations in the current. If the current is given by: I=Ni, where N is a measure of the electrolyte concentration and i is the current carried by a single ion, then assuming that the ions are statistically independent, it must be the mean square fluctuations in the current are ( [symbol] ΔI²>)=N [symbol] Δi² [symbol] . Therefore, by substitution, [symbol] ΔI² [symbol] /I²˜N/N²=I/N, from which it follows that S_{l/f}/I²₀˜1/N, which is consistent with all the data shown in FIG. 3a for I₀≤1 pA, where the scaled noise power S_{l/f}/I²₀>10⁻³ Hz⁻¹. However, for higher currents in sub-nanopores with cross-sections smaller than 0.41 nm², the scaled noise power, S_{l/f}I²₀, was observed to be independent of the current, signaling the development of correlations in the current fluctuations (FIG. 3a). In other words, the variance no longer scales trivially with the concentration. Instead, as observed empirically, the mean square fluctuations could be described by: ( [symbol] ΔI² [symbol] =N²) [symbol] ΔI² [symbol] , so that S_{l/f}/I²˜1.

Correlations in the noise like this have been observed in much larger pores with larger electrolyte carrying the current, and attributed to a (traffic) “jamming” transition in which congestion between the carriers develops at high current gives rise to non-linear density waves and bunching of the ions in the pore.⁽⁴⁾ The scale for the transition was associated with the carrier size, which should be comparable to a hydrated sodium ion, 0.21 nm, in a sub-nanopore filled with NaCl electrolyte. Consistent with this attribution, as the pore cross-section was reduced, it was observed that the threshold current for the jamming transition was also reduced from Iₜ=70 pA for 0.21 nm² to Iₜ-1.5 pA at 0.12 nm² with no threshold observed for pores with a cross-section ≥nm² (FIG. 3b). Taken altogether, these data support the contention that, beyond a threshold affected by the pore cross-section, correlations develop in the current.

Correlated noise has been reported to induce a directed current that exhibits a resonant-like behavior.^{(25, 26)} For example, non-equilibrium fluctuations can support a stationary current in an asymmetric periodic, “ratchet” potential. These notions are related to stochastic resonance, which occurs in nonlinear systems with a threshold.⁽²⁷⁾ So, it was inferred that correlated noise in the blockade current might improve the read fidelity associated with the occluded volume of a quadromer. Furthermore, it was inferred that increasing the occluded volume, should decrease the jamming threshold and improve the SNR. To test these hypotheses, the data acquired as a single H3.2 molecule was impelled through a sub-nanopore frictionlessly on a force plateau was segmented according to the force ACF, where minima in the force were identified with a single quadromer affecting the blockade current predominately (FIG. 3c). The rms-variation in the segmented data was subsequently analyzed to discover that the standard deviation diminishes with increasing pore volume (FIG. 3d) as expected. Further analysis of the fluctuations as a function of frequency revealed that the frequency content, measured by the ratio of the low-to-high frequency components to the fluctuating signal increases with the fractional blockade, which indicates that the enhancement was nonlinear and became more pronounced with increasing fractional blockade (FIG. 3e).

The forces and blockade currents characterizing the translocation of a single protein molecule tethered to the tip of an AFM cantilever were measured as the molecule was impelled systematically through a sub-nanopore. Autocorrelations of the force and current fluctuate periodically with a lag that corresponded to the equilibrium separation between AA residues, and the amplitude of the current fluctuated in proportion to the occluded volume of quadromers in the protein sequence. Since the sub-nanometer cross-section of the pore was comparable to the size of a hydrated ion, correlations also developed in the current noise in a sub-nanopore, which enhance the SNR associated with the occluded volume of an AA residue.

Methods:

Sub-nanopore Fabrication. Pores with sub-nanometer cross-sections were sputtered through thin silicon nitride membranes using a tightly focused, high-energy electron beam carrying a current ranging from 300-500 pA (post-alignment) in a scanning transmission electron microscope (STEM, FEI Titan 80-300, Hillsboro, Oreg.) with a Super-TWIN pole piece and a convergence angle of 10 mrad. For example, a 398 pA beam was used to sputter a nominally 0.3-nm-diameter pore in 50 sec. The silicon nitride was deposited by LPCVD directly on the top surface of a polished silicon handle wafer and a membrane was revealed using an EDP (an aqueous solution of ethylene diamine and pyrocatechol) chemical etch through a window on the polished back-side of the handle. The thickness of the membranes, which ranged from t=8 to 12 nm, was measured in situ using electron energy loss spectroscopy (EELS) prior to sputtering. The roughness of the membrane, measured with custom-built silicon cantilevers (Bruker, Fremont, Calif.) with 2 nm radius tips, was estimated to be <0.5 nm-rms.

Multi-slice Image Simulations. TEM images of the pores were simulated using the Dr. Probe software package.⁽⁴²⁾ The simulation procedure started by creating an atomistic model of the structure. An approximation to an amorphous Si₃N₄ membrane was created by randomly filling a tetragonal 5×5×10 nm³ (x-y-z) cell with Si and N atoms. The total number of atoms was determined by the volume (250 nm³), the density of stoichiometric Si₃N₄ (3.44 g/cm³) and the molecular weight of Si₃N₄ (140.28 g/mol). Atoms that were closer together than 0.16 nm were removed from the structure. In order to create a bi-conical pore with an elliptical cross-section at the waist, atoms were selectively extracted from the volume within a border defined by the following mathematic model:

{square root over ((x−2.5+a))²+(y−2.5))}²+{square root over ((x−2.5+a))²+(y−2.5)²=2a)}, where

a=aₒ+tan(a) z−5|, b=bₒ+tan(a)z−5|, and c={square root over ((a²−b²)} and where x, y, and z denote the coordinates of each atom (in nm), a is the cone angle, a/b are the major and minor axes of the ellipse and c is the eccentricity.

To prepare the model structures for the calculation of dynamic electron diffraction by means of the multislice algorithm,⁽⁴³⁾ the input cells were partitioned into 40 equidistant slices along z. Phase gratings of the slices were calculated on grids with 512×512 pixels in x and y for 300 keV incident electrons using the elastic and absorptive form factors and Debye-Waller factors to account for the thermal motion of the atoms.⁽⁴⁴⁾ The multislice calculations yielded an exit-plane wavefunction consistent with the specified model of the structure. Based on the exit-plane wave functions, TEM images were constructed using a phase contrast transfer function consistent with the microscope and defocus, assuming instrumental parameters such the aberration coefficient (Cₛ=0.9 mm) and the aperture size of the objective (150 μm) at an acceleration voltage of 300 kV. Because the defocus was uncertain, a defocus series from −50 to 50 nm was calculated for comparison to the actual images. The TEM image calculations account for the partial temporal coherence with a focus-spread of about 4 nm and for the partial spatial coherence with a 0.4 mrad semi-angle of convergence.

Microfluidics. A silicon chip supporting a single membrane with a single pore through it was bonded to a polydimethylsiloxane (PDMS, Sylgard 184, Dow Corning) microfluidic device formed using a mold-casting technique. The PDMS microfluidic was formed from a thoroughly stirred 10:1 mixture of elastomer(siloxane) with a curing agent (cross-linker) cast in a mold formed from DSM Somos ProtoTherm 12120 plastic (Fineline Prototyping, Raleigh, N.C.) and then degassed and cross-linked at 75° C. for 2 hr. The microfluidic device consisted of two microchannels (each 250×75 μm2 in cross-section) connected by a via 75 μm in diameter. The small via was created using a fine needle to penetrate a thin PD MS layer immediately above the pore. The diameter of the via was measured relative to a micrometer calibration grid (Ted Pella, inc) in an inverted optical microscope (Zeiss Observer Z1). The small via has the benefit of reducing the parasitic capacitance due to the silicon handle wafer supporting the silicon nitride membrane and thereby diminishing the dielectric component of the electrical noise.

A tight seal was formed between the silicon chip containing the silicon nitride membrane with the pore in it and the PDMS trans-microfluidic channel with a plasma-bonding process (PDS-001, Harrick Plasma, Ithaca, N.Y.). The membrane with a pore through it was plasma-bonded to the trans-side of the PDMS microfluidic using a (blue-white) 25 W oxygen plasma (PDS-001, Harrick Plasma, Ithaca, N.Y.) for 30 sec. The cis-channel was likewise sealed to a clean 75×25 mm² glass slide, 1 mm-thick (VWR, Radnor, Pa.) using the same bonding strategy. To ensure a >100 GΩ seal to the PDMS, the backside of the silicon chip was painted with PDMS, and then the ensemble was heat-treated at a temperature of 75° C. for 30-60 min. Subsequently, two separate Ag/AgCl electrodes (Warner Instruments, Hamden, Conn.) were embedded in each channel to independently electrically address the cis and trans-sides of the membrane. Likewise, the two microfluidic channels were also connected to external pressure and fluid reservoirs through polyethylene tubing at the input and output ports. The port on the cis-side was used to convey proteins to the pore. Finally, the sealing protocol was tested against a nominally 10 nm thick silicon nitride membrane without a pore in 200 mM NaCl pH 7.5 for >4 weeks without failure; the leakage current was <12 pA at 1V.

Low-noise electrical measurements. To perform current measurements, a pore with a sub-nanometer cross-section was immersed in 200-300 mM NaCl electrolyte and allowed to stand for 1 da (typically) to wet the pore, a transmembrane voltage was applied using Ag/AgCl electrodes and the corresponding pore current was measured at 22±0.1° C. using an Axopatch 200B amplifier with the output digitized with DigiData 1440 data acquisition system (DAQ, Molecular Devices, Sunnyvale, Calif.) at a sampling rate of 100-250 kHz. Clampex 10.2 (Molecular Devices, Sunnyvale, Calif.) software was used for data acquisition and analysis.

To measure a blockade current, a bias ranging from −0.3 V to −1 V was applied to the reservoir containing 200 μL of electrolytic solution and 5 μL of 2×10⁻⁴% (v/v) SDS (denaturant) along with 32 nM protein relative to ground in the channel. The background noise level was typical 1 y 12 pA-rms i n 250 mM NaCl solution at −0.7 V. Recombinant, carrier-free protein was reconstituted according to the protocols offered by the manufacturer (R& D Systems). Typically, the protein was reconstituted at high (10 μg/ml) concentration in PBS without adding BSA to avoid false readings. From this solution, aliquots diluted to 10× the concentration of denaturant with 32 nM protein in 250 mM of NaCl electrolyte, 100 μM BME, and 0.01% SDS were vortexed and heated to 75° C. for 15-60 min. The solution was allowed to cool and then the AFM tips, decorated with BSA-SA, were immersed in 100 μL of the denatured protein solution for 45 min before use.

Force Spectroscopy with an Atomic Force Microscope: The force and current data were obtained on a customized AFM (MFP-3D-BIO, Asylum Research, Santa Barbara, Calif.) interfaced to an inverted optical microscope (Axio-Observer Z1, Zeiss). In particular, the AFM employed a narrow bandwidth filter (850 nm-center±30 nm pass-band with >OD 6 out-of-band) for the superluminescent diode in the head and a low noise Z-sensor coupled with an ultra-quiet Z-drive to produce noise in the tip-sample distance <30 pm at 1 kHz bandwidth. To minimize drift and reduce acoustic noise, the inverted optical microscope was mounted on an optical air table with active piezoelectric vibration control (Stacis, TMC, Peabody, Mass.), housed in an acoustically isolated, NC-25 (Noise criterion) rated room in which the temperature was stabilized to less than ±0.1° C. over 24 h through radiative cooling. Temperature fluctuations appear to be the dominant source of long-term drift, and with temperature regulation the drift of the system was reduced to 600 pm/min. Sound couples strongly into the microscope and is another potential source of instrument noise. Therefore, acoustically loud devices, especially those with cooling fans such as power supplies, amplifiers, and computers, were placed outside of the room. With these precautions, force detector noise is <10 pm/A/Hz for frequencies above 1 Hz; the on-surface positional noise measured <45 pm A-dev.

The Z-piezo sensor (Z-sensor) was calibrated using a standard calibration grating (NT-MDT, Moscow, Russia). The deflection sensitivity was calibrated by pressing the tip against a freshly cleaved mica surface and correlating the cantilever deflection to the Z-sensor reading. The spring constant was determined by measuring the thermal noise spectra and fitting the response to a simple harmonic oscillator.⁽²⁸⁾

The topography of the silicon nitride membrane and the location of the pore relative to the edges of the membrane were determined in air in non-contact (tapping) mode using silicon cantilever (SSS-FM, Nanosensors, Neuchatel, Switzerland) with a 2 nm nominal radius, and a spring constant ranging from 0.5-9.5 nN/nm and a 45-115 kHz resonant frequency (in air). Force spectroscopy was performed in 250 mM NaCl, using either contact mode cantilevers (PPP-CONT, Nanosensors) with a 7 nm nominal tip radius, a 0.02-0.8 nN/nm spring constant and a 6-21 kHz resonance frequency, or custom MSNL silicon cantilever (Bruker, Camarillo, Calif.) without metal reflex with a 2 nm tip radius, 0.005-0.3 nN/nm spring constant, and a 4-100 kHz resonant frequency. Considering only the off-resonance thermal noise of the cantilever ΔFmin=\sqrt{4 k_{B}TΔf kₛₚᵣ/ωₒQ<3 pN}, where typically kₛₚᵣ=10-30 pN/nm, Δf=100-250 Hz is the measurement bandwidth, ωₒ=2π×1.2-22.7 kHz is the angular resonance frequency of the cantilever, and Q=1-1.5 is the quality factor.

To functionalize an AFM tip, the cantilever was first conditioned in a 20% oxygen plasma at 25 W (Harrick Plasma) for 1 min and then immersed in a 0.1% (v/v) solution of 3-aminopropyltriethoxysilane (APTES, Sigma) and deionized water (18.2 Mn Millipore, Billerica, Mass.) for 5 min followed by a rinse in deionized water. The cantilever was then exposed to biotin labeled bovine serum albumin (BSA, 1 μg/ml, Sigma) in a phosphate buffer saline solution (PBS, pH 7.4) for 45 min, rinsed with PBS and stored at −20° C. for up to 7 days until used. Prior to force spectroscopy measurements, the tips were placed in 100 μl of streptavidin (0.1 μg/ml, S4762, Sigma-Aldrich) in PBS for 45 min at 20° C., rinsed in PBS and then immersed in denatured 100 nM H3.2 in PBS and incubated for another 45 min at 20° C. followed by a final rinse in 250 mM NaCl electrolyte before mounting on the cantilever holder. The force on the frictionless plateaus and rupture force associated with the “slip-stick” transitions are smaller than that required to rupture either the streptavidin-biotin⁽²⁹⁾ or the non-specific bond between BSA to silicon.⁽³⁰⁾

After a topographical scan, with the membrane immersed in electrolyte, the location of the pore was reacquired in either constant force mode (contact mode) or in tapping mode by triangulation using the corners of the membrane and high-resolution topology maps imaged with an unfunctionalized tip. In this way, the pore can be located with a functionalized tip with minimal scanning thus preserving the protein on the tip. To measure the force between the pore and protein, the functionalized tip was positioned 30-150 nm above the pore and extended toward and retracted from the membrane at 10-20 nm/s and 4 nm/s respectively, with an applied voltage bias while the current, tip deflection and Z-position were recorded. Contact with the surface resulted in typical tip deflections of 5-10 nm representing applied forces of 50-500 pN. In addition, to increase the sensitively, lock-in detection was also used to measure the deflection (5210, Signal Recovery, Oak Ridge, Tenn.) in response to a 100 μVₚₚ reference signal ac-coupled to the dc trans-membrane bias voltage.

Measurements of the Current Blockade with Protein Tethered to the AFM: The ionic current through a nanopore was measured several ways using either: 1. a current-sensing trans-impedance amplifier with a gain of 1×10⁶ V/A connected directly to the cantilever holder (Orca, Asylum Research); 2. a patch-clamp amplifier (Axopatch 200B, Molecular Devices) in whole-cell mode; and 3. phase-sensitive lock-in detection (Signal Recovery 5210—Stanford Research) in response to minute periodic changes in the electric field in the nanopore. In each case, A g/AgCl electrodes embedded in the microfluidic device were used to establish a trans-membrane potential and monitor the pore current. For lock-in detection, the applied dc bias was combined with an ac-signal (100 μVpp) voltage that was used as a reference signal. Each data channel was subsequently digitally filtered at 5 kHz and sampled at 10 kHz and then digitally filtered again using a 100 Hz eight pole Bessel filter (MATLAB).

Signal autocorrelation function: Noise in the z-positional sensor results in multiple measurements for each unique position. Thus, all time series were binned at unique Z-positions spaced every 25 pm and the mean within each bin was calculated. The spatial autocorrelation (ACF) of the signal S_{z}={S₁, S₂, . . . , S_{N}} at lag k was calculated from: ACFₖ, =1/N Σ (S_{z}−S)(S_{z+k}−S) where S represents the mean signal.

Signal estimation. Data handling has been described elsewhere.⁽³⁾ Briefly, current blockades were manually cropped from the traces based on the start and end positions (in time) of the force plateau. As the ACF analysis already determined the AA periodicity (p), it was possible to infer the total number of AAs (N) for a blockade of duration, t, as equal to N=Vt/p. Further, because the end of the force blockade was associated with the free end of the molecule (the C-terminus—i.e. the opposite of the N-terminal biotin tip-side bond), the blockade ranged from the AA position (AAₘₐₓ−N) to the C terminus itself.

Assuming the peaks were periodic in time, the blockade was linearly re-sampled into N bins. The model developed for occluded volume shows variations in nm³ as a function of position. However, the events were recorded in units of pA. The scaling of pA to nm³ was necessary to compare the model and the consensus events, and can be directly inferred using both the pore geometry and open pore current. However, we chose to linearly scale the ordinate of events to the volume model using a Nelder-Mead method search. Errors were measured as the percentage difference in these two normalized traces. Regions of error were coded with different gray-tones. If the error was greater than a given threshold, E=|(C_{1 . . . n}−V_{1 . . . n})|V_{1 . . . n}≥T, it was indicated as black and elsewhere it was indicated as gray when considered consistent with the model. Similarly, errors as a function of read position were found by contributing the vector of errors (as described above) at each site to all possible AAs recorded at that read position. For example, consider a single event where an error of 6% on read position 5 could have arisen from any part of the position trimer {4, 5, 6}. After these assignments, all possible errors on every AA were then summed and normalized to the total observed error on the event and plotted as a function of AA and AA volume for each protein.

Finite Element Simulations: Finite element simulations (FESs) of vacated (open) pores, which ignored the atomistic details of the structure and electrolyte, were used to examine the distribution of the electrostatic potential and current. They were described elsewhere.⁽³⁾ Briefly, FESs of the electric field and the electro-osmotic flow were performed using COMSOL (v4.2a, COMSOL Inc., Palo Alto, Calif.), following a Poisson-Boltzmann formalism described elsewhere.⁽³¹⁾ Briefly, the applied potential ϕ and the potential ψ due to charges in the pore are decoupled from one another and solved independently. The relationship between φ and the charge carriers, Na⁺ and Cl⁻, is given by the Poisson equation, ∇φ6−ρ/εεₒ, where ρ, ε, and εₒ are the volume charge density, and the relative and vacuum permittivities respectively. The charge density is given by ρ=FΣzᵢcᵢ, where F=96,485 C/mol is the Faraday constant, zᵢ is the valence and cᵢ is the molar concentrations of ionic species i. The distribution of ions close to charged surfaces satisfies the Boltzmann distribution; thus, the charge density is given by cᵢ=c_{o,i} exp(−zᵢeψ/k_{B}T), where cₒ is the molar concentration far from the sub-nanopore (i.e. bulk concentration), e is the electric charge, k_{B}=1.38×10⁻²³ J/K is the Boltzmann constant, and T=298 OK is the temperature.

Electro-osmotic flow is expressed by the Navier-Stokes equation, n∇2u−∇p−FΣzᵢcᵢ ∇V=0, where V=φ+ϕ, n, p is the pressure, and u is the velocity. The transport of ionic species is described by the Nernst-Planck equation given by Dᵢ∇²cᵢ+zᵢ, μᵢcᵢ∇²V=u∇cᵢ, where Dᵢ is the diffusion coefficient and μᵢ is the ionic mobility of the i^{th} species. In this treatment, u, V and ci are coupled between equations. The relationship between the surface charges σₛ and the zeta potential ζ is given by the Grahame equation:⁽³²⁾

σ̅ₛ̅(̅ζ̅)̅=̅√̅8cₒεεₒk_{B}T sin h(eζ/2k_{B}T).

Table 1 A and 1B. Components of the models for protein sequencing based on residue volumes. AA volumes taken from S. J. Perkins, Eur. J. Biochem., 157, 169-180 (1986) and the sequences for H3.2N and IgG4 taken from publically available uniprot genetic data bases (P13501—CCL Human)(P02769—Albumin-Bovine)(P09341—GROA Human)(P69432—(H3 Human).

```
TABLE 1A-H3.2
                10 20 30 40
MARTKQTARK STGGKAPRKQ LATKAARKSA PATGGVKKPH
                50 60 70 80
RYRPGTVALR EIRRYQKSTE LLIRKLPFQR LVREIAQDFK
                90 100 110 120
TDLRFQSSAV MALQEASEAY LVGLFEDTNL CAIHAKRVTI
              130
MPKDIQLARR IRGERA
```

TABLE 1B

| | AA volume | |
|---|---|---|
| AA | (nm³) | |
| I | 0.1688 | |
| F | 0.2034 | |
| V | 0.1417 | |
| L | 0.1679 | |
| W | 0.2376 | |
| M | 0.1708 | |
| A | 0.0915 | |
| G | 0.0664 | |
| C | 0.1056 | |
| Y | 0.2036 | |
| P | 0.1293 | |
| T | 0.1221 | |
| S | 0.0991 | |
| H | 0.1673 | |
| E | 0.1551 | |
| N | 0.1352 | |
| Q | 0.1611 | |
| D | 0.1245 | |
| K | 0.1713 | |
| R | 0.2021 | |
| CCL5: | 1 | SPYSSDTTPC |
| | 11 | CFAYIARPLP |
| | 21 | RAHIKEYFYT |
| | 31 | SGKCSNPAVV |
| | 41 | FVTRKNRQVC |
| | 51 | ANPEKKWVRE |
| | 61 | YINSLEMS |
| CXCL1: | 1 | ASVATELRCQ |
| | 11 | CKQTKQGIHP |
| | 21 | KNIQSVNVKS |
| | 31 | PGPHCAQTEV |
| | 41 | IATLKNGRKA |
| | 51 | CLNPASPIVK |
| | 61 | KIIEKMLNSD |
| | 71 | KSN |
| BSA: | 1 | DTHKSEIAHR |
| | 11 | FKDLGEEHFK |
| | 21 | GLVLIAFSQY |
| | 31 | LQQCPFDEHV |
| | 41 | KLVNELTEFA |
| | 51 | KTCVADESHA |
| | 61 | GCEKSLHTLF |
| | 71 | GDELCKVASL |
| | 81 | RETYGDMADC |
| | 91 | CEKQEPERNE |
| | 101 | CFLSHKDDSP |
| | 111 | DLPKLKPDPN |
| | 121 | TLCDEFKADE |
| | 131 | KKFWGKYLYE |
| | 141 | IARRHPYFYA |
| | 151 | PELLYYANKY |
| | 161 | NGVFQECCQA |
| | 171 | EDKGACLLPK |
| | 181 | IETMREKVLA |
| | 191 | SSARQRLRCA |
| | 201 | SIQKFGERAL |
| | 211 | KAWSVARLSQ |
| | 221 | KFPKAEFVEV |
| | 231 | TKLVTDLTKV |
| | 241 | HKECCHGDLL |
| | 251 | ECADDRADLA |
| | 261 | KYICDNQDTI |
| | 271 | SSKLKECCDK |
| | 281 | PLLEKSHCIA |
| | 291 | EVEKDAIPEN |
| | 301 | LPPLTADFAE |
| | 311 | KDKVCKNYQE |
| | 321 | AKDAFLGSFL |
| | 331 | YEYSRRHPEY |
| | 341 | AVSVLLRLAK |
| | 351 | EYEATLEECC |
| | 361 | AKDDPHACYS |
| | 371 | TVFDKLKHLV |
| | 381 | DEPQNLIKQN |
| | 391 | CDQFEKLGEY |
| | 401 | GFQNALIVRY |
| | 411 | TRKVPQVSTP |
| | 421 | TLVEVSRSLG |
| | 431 | KVGTRCCTKP |
| | 441 | ESERMPCTED |
| | 451 | YLSLILNRLC |
| | 461 | VLHEKTPVSE |
| | 471 | KVTKCCTESL |
| | 481 | VNRRPCFSAL |
| | 491 | TPDETYVPKA |
| | 501 | FDEKLFTFHA |
| | 511 | DICTLPDTEK |
| | 521 | QIKKQTALVE |
| | 531 | LLKHKPKATE |
| | 541 | EQLKTVMENF |
| | 551 | VAFVDKCCAA |
| | 561 | DDKEACFAVE |
| | 571 | GPKLVVSTQT |
| | 581 | ALA |
| H3N: | 1 | ARTKQTARKS |
| | 11 | TGGKAPRKQL |

```
UniProtKB-P01861 (IGHG4_HUMAN)
                10 20 30 40
ASTKGPSVFP LAPCSRSTSE STAALGCLVK DYFPEPVTVS
                50 60 70 80
WNSGALTSGV HTFPAVLQSS GLYSLSSVVT VPSSSLGTKT
                90 100 110 120
YTCNVDHKPS NTKVDKRVES KYGPPCPSCP APEFLGGPSV
              130 140 150 160
FLFPPKPKDT LMISRTPEVT CVVVDVSQED PEVQFNWYVD
              170 180 190 200
GVEVHNAKTK PREEQFNSTY RVVSVLTVLH QDWLNGKEYK
              210 220 230 240
CKVSNKGLPS SIEKTISKAK GQPREPQVYT LPPSQEEMTK
              250 260 270 280
NQVSLTCLVK GFYPSDIAVE WESNGQPENN YKTTPPVLDS
              290 300 310 320
DGSFFLYSRL TVDKSRWQEG NVFSCSVMHE ALHNHYTQKS
LSLSLGK
MGSSHHHHHH SSGLVPRGSH MASMTGGQQM GRGSEF-
QPREPQVYT LPPSQEEMTK NQVLSTCLVK GFYPSDIAVE
WESNGQPENN YKTTPPVLDS DGSFFLYSRL TVDKSRWQEG
NVFSCSVMHE ALHNHYTQKS LSLSLGK
```

### Example 2—Detecting the Sequence of Amino Acid Quadromers in Protein Molecules Using a Sub-Nanometer-Diameter Pore

The primary structure of a protein consists of a sequence of amino acids (AAs) that essentially dictates how the protein folds and functions. Here, it is shown that the sequence of AA quadromers in a denatured protein molecule can be determined using a pore with a sub-nanometer diameter (a sub-nanopore) in a thin inorganic membrane. When a sub-nanopore is immersed in electrolyte and a voltage is applied across it, measurements of a blockade in the current, associated with the translocation of a protein molecule, reveal nearly regular fluctuations, the number of which coincides with the number of residues in the protein. Furthermore, the amplitudes of the fluctuations are highly correlated with the volumes occluded by quadromers (four AA residues) in the protein sequence. Scrutiny of the fluctuations reveal that a sub-nanopore is sensitive enough to detect the occluded volume related to chemical modifications at a single residue within a quadromer. Thus, each fluctuation represents a read of a quadromer. Although the read fidelity is low, it is more than double the accuracy of electrical noise. Thus, with sufficient coverage, this methodology could augment the short reads offered by techniques such as mass spectrometry with long reads for protein quantitation.

Here, it is shown that the sequence of AA quadromers in a denatured protein amino acid sequence can be determined by measuring the electrolytic current through a pore with a sub-nanometer cross-section. When the pore is immersed in electrolyte containing denaturant and an electric field is applied across it, measurements of a blockade in the current, associated with the translocation of a single protein molecule, reveal nearly regular fluctuations, the number of which coincides with the number of amino acid residues in the protein. Each fluctuation represents a read of a quadromer (four AA residues) located in the waist of the pore near the center of the membrane. Furthermore, it is shown that the amplitude of each fluctuation is highly correlated with the volume occluded by a quadromer in the protein sequence, which means that the sequence could be identified by measurements of the blockade current.

Nanopores have not been used for sequencing proteins. Studies of unfolded polymers translocating through the pore lumen have been reported. However, these reports fail to provide acceptable approaches to sequencing of proteins because, among other reasons, the secondary and higher order structure of the protein confounds the interpretation of the blockade current and overwhelms the chemical specificity. To recover the signal-to-noise ratio, a technique using smaller pores and denatured amino acid sequences of a protein is needed. Moreover, because the charge distribution along the native protein is not uniform, the systematic control of the translocation kinetics by the electric field in the pore is frustrated.⁽⁴⁹⁾ Instead, in conjunction with an electric field, enzymatic motors have been used to drive proteins stochastically through a pore by repeatedly pulling on the substrate protein to unfold it. While protein domains have been identified this way, this approach fails to identify AA residues.

The development of nanopores for sequencing has focused mainly on DNA.^{(50, 51)} Nanopore sequencing of DNA is distinguished from all the other methodologies by kilo-base long reads of single molecules.⁽⁵²⁾ However, single nucleotide resolution demands sub-nanometer control over both the molecular configuration in the pore and the translocation kinetics because the equilibrium distance between nucleotides is only 0.35 nm. Biological nanopores satisfy these criteria. In particular, the biological pore, MspA, conjugated with a polymerase (phi29) that steps the DNA through it, has been used to sequence with 4.5 kb long reads in which 4 nucleotides affect the ion current of each blockade level. Similarly, MinION™ commercialized by Oxford Nanopore, uses a motor enzyme, in combination with an electric field, to drive a single DNA molecule through a variant of a-hemolysin biological pore to sequence with 8-10 kb long reads in which 5 nucleotides affect the ion current of each blockade level. Although the fidelity of the reads is low—the Oxford v7 chips show only about a 68% correct per-read average—with high coverage (30×) MinION is a practicable DNA sequencer. However, these methodologies for sequencing DNA cannot be easily extended to protein because the pores are too large—lacking chemical specificity—and the chemical agents needed for denaturation would adversely affect a biological nanopore.

Results and Discussion: To sequence protein with a nanopore, several technical hurdles have to be overcome. First, the protein has to be denatured to eliminate the higher-order structure and facilitate the interpretation of the blockade current associated with AAs.⁽⁵³⁾ Second, the deficient chemical sensitivity of a pore (which may be related to the volume occluded by the molecule, the charge distribution and the dependence of the monomer mobility)⁽⁵⁴⁾ has to be improved. Third, if an electric force field in the pore is to be used to drive the molecule through the pore systematically, the charge distribution along the protein has to be uniform.

To overcome these hurdles, sub-nanopores sputtered through thin inorganic silicon nitride membranes were used to analyze single proteins denatured by heat, sodium dodecyl sulfate (SDS) and β-mercaptoethanol (BME). The precise control exercised over the pore topography by electron beam-induced sputtering⁽⁵⁵⁾ in a scanning transmission electron microscope (STEM) was the linchpin affording us the opportunity to make pores smaller than the size of an a-helix (which has a diameter <0.5 nm and a rise of 0.56 nm), a common secondary structure found in a protein, and comparable to the size of a hydrated ion⁽⁵⁶⁾ (FIGS. 5a-c,i). The small size was the key to improved chemical specificity.

The topographies of the sub-nanopores were inferred from TEM. Since the information limit of the microscope was 0.11 nm, to accurately assess the topography, each micrograph was imitated by multi-slice simulations (FIGS. 5a-c,i i). The simulations reproduced the actual imaging conditions, while accounting for dynamic scattering of the electron beam by the membrane. The close correspondence between the images and simulations signified that the models (FIG. 5a-c,iii-iv) were realistic representations of the actual pores. From the simulations, it was inferred that the pores were bi-conical, with cone-angles that ranged around θ=15±5°, and irregular, with cross-sections at the waist that ranged from d=(0.4±0.1)×(0.5±0.1) nm² to (0.7±0.1)×(0.8±0.1) nm². Measurements of the transmission through a pore as a function of the tilt angle, i.e. the “wink-out” angles, were used to confirm the cone angles (FIG. 5).

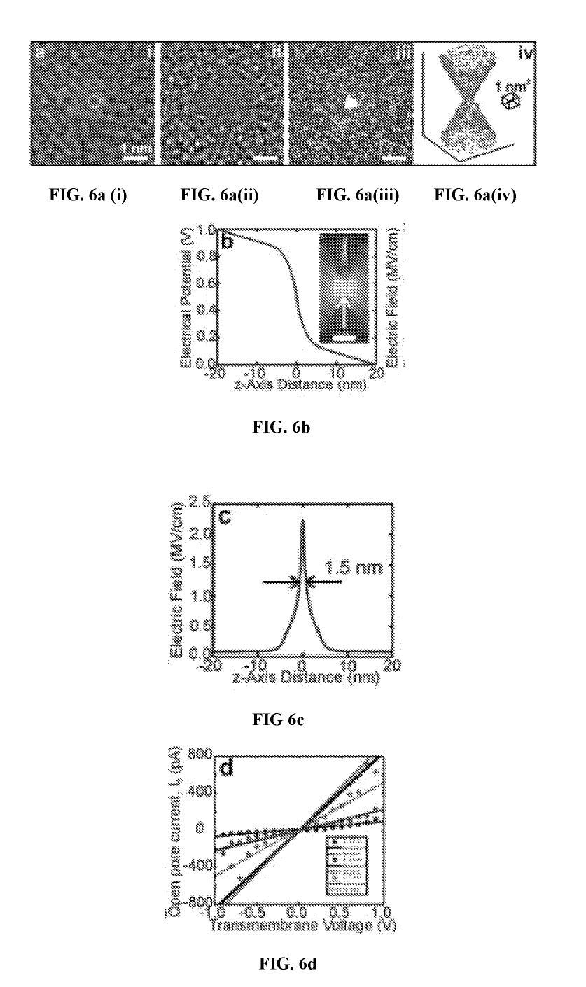

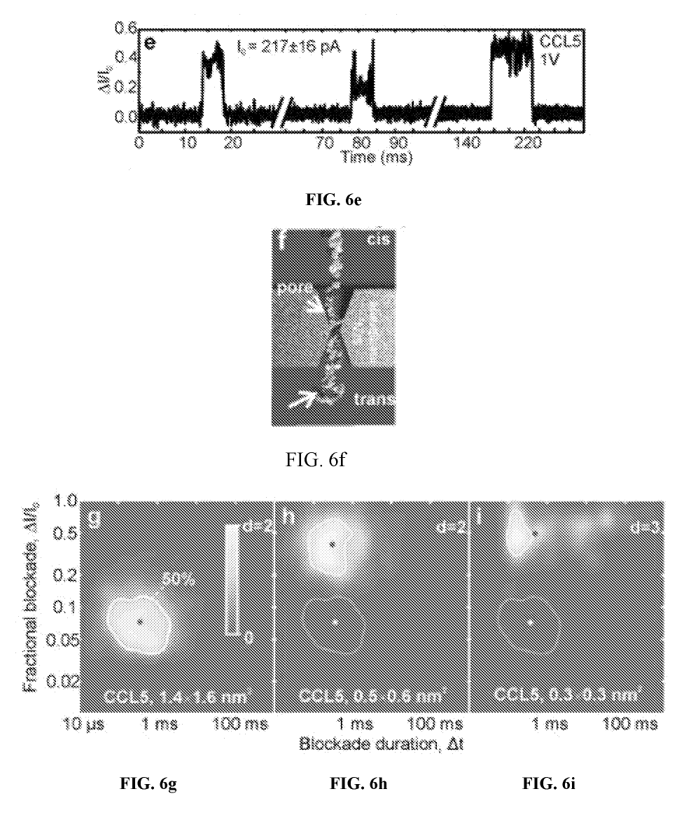

The electrolytic conductance measurements along with finite element simulations (FES) of them provided additional corroborative evidence of the pore size (FIGS. 6a-d), after accounting for pore charge (FIG. 6). The accuracy with which FES captured the measured conductances of sub-nanopores supports the models derived from TEM images (FIG. 6d). Furthermore, FES of the pores revealed that the bi-conical topography crowds the current and focuses the electric field at the waist into a region about 1.5 nm in extent (FIG. 6c), which is approximately four AA residues long (since there is about 0.36 nm per AA on the peptide chain.)

Another aspect of this method for analyzing protein involves the use of thin, silicon nitride membranes that are resistant to chemical agents like SDS and BME, and the high temperature used for denaturation. SDS is an anionic detergent that works, in combination with heat (45-100° C.) and reducing agents like BME, to impart a nearly uniform negative charge to the protein that stabilizes denaturation. Although the aggregates formed by SDS and proteins have been investigated extensively, the exact structure remains unsolved.⁽⁵⁷⁻⁶³⁾ It is likely dependent on the protein and the SDS concentration. Several models have been proposed for the aggregate.⁽⁶²⁾ A “rod-like” model in which the SDS molecules form a shell along the length of the protein backbone was adopted here.⁽⁵⁷⁾ The resulting uniform charge on the protein offered the benefit of facilitating electrical control of the translocation kinetics.

Six types of protein were analyzed using sub-nanopores: two recombinant human chemokines with a similar molecular weight, RANTES (CCL5, 7.8 kDa MW) and CXCL1 (8 kDa MW); bovine serum albumin (BSA, 66.5 kDa MW) with a much higher molecular weight; and three biotinylated, subtly different variants of the tail peptides (residues 1-20) of hi stone H3. These variants of H3 are involved in the chromatin structure in eukaryotes. One of the peptides was native (denoted by H3N, 2.5 kDa) and the other two were chemically modified at a single position 9 (lysine) either by acetylation (H3A) or trimethylation (H3M). Typically, when a dilute concentration (300 pM) of denatured protein with SDS and BME was introduced on the cis-side of a pore, blockades were observed in the open pore current (FIG. 5d), which were attributed to the translocation of single molecules (FIG. 5e). Generally, no blockades were observed beyond the noise in controls that comprised the electrolyte and the denaturants (SDS and BME), which were heated to 75° C. and then cooled without protein. Infrequently, short duration events were observed in the controls, but these were easily culled due to the band-limited duration of the blockade. To facilitate comparisons and diminish the dependence of the pore current on voltage and electrolytic conductance, the distribution of blockades was classified by the fractional change in the pore current relative to the open pore value (ΔI/I₀) and the duration of the blockade (Δt).

Naively, the fractional change in current can be related to the ratio of the molecular volume to the pore volume: i.e. ΔVₘₒₗ/Vₚₒᵣₑ·(7) For example, native CCL5 has 68 AA residues, so if the denatured protein has a rod-like shape, about 22 AAs would span the entire membrane and the occluded volume would be about Vₘₒₗ=3.5 nm³. A pore 1.5 nm in diameter with θ=15° in an 8 nm thick membrane has an estimated open volume of Vₚₒᵣₑ=43.8 nm³ so that for a denatured protein, ΔI/I₀=0.0698. This estimate is a lower bound since it neglects features such as the hydration shell and a persistent, native-like topology. Moreover, if the effective thickness of the membrane is defined by the current crowding associated with the bi-conical topography, only four AAs would span a thickness of 1.5 nm, and so the occluded volume would be only about ΔVₘₒₗ/V^{eff}ₚₒᵣₑ=0.64 nm³/3.4 nm³=0.19˜ΔI/I₀. The expectations improve with a 0.5 nm-diameter pore with a cone angle of 15° in an 8-nm thick membrane, which has an estimated volume of only Vₚₒᵣₑ=18.1 nm³, so that ΔI/I₀˜0.169 if the protein spans the entire membrane. On the other hand, if only that portion of the membrane where the current is crowded is taken into account, then ΔI/I₀˜0.94 instead. Furthermore, for a 0.3-nm-diameter pore, the estimated volume would be Vₚₒᵣₑ=14.2 nm³ if the protein spans an 8 nm thick membrane, so that the fractional blockade should improve to ΔI/I₀˜0.216. However, the estimated volume would be only Vₚₒᵣₑ=0.31 nm³ if the signal develops mainly in the waist of the pore, so that the fractional blockade would be maximal ΔI/I₀). In summary, 0.07<ΔI/I₀<0.19, 0.17<ΔI/I₀<0.94, and 0.22<ΔI/I₀<1 for pores with a 1.5-, 0.5- and 0.3-nm-diameter.

These expectations were borne out in heat maps derived from the ionic blockade distributions associated with CCL5 translocations collected from different pores: one with a (1.4±0.1)×(1.6±0.1) nm² cross-section; another with (0.5±0.1)×(0.6±0.1) nm² cross-section; and a third with a (0.3±0.1)×(0.3±0.1) nm² cross-section (FIGS. 5f,g). For a 1V bias, the median fraction ΔI/I₀=0.07 occurs at a median duration of about Δt=400 μs for the 1.4×1.6 nm² pore. On the other hand, for the same protein, the median blockade in the 0.5×0.6 nm² cross-section pore improves substantially to: ΔI/I₀=0.38, but occurs at nearly the same median duration of about Δt=330 μs. Moreover, although the distributions broaden, the median duration hardly changed with diameter. In particular, for the 0.3 nm-diameter pore, the blockade distribution extended over a range from 100 μs<Δt<70 ms and 0.25<ΔI/I₀<1. The median fraction improved to ΔI/I₀=0.47 with a median duration of about Δt=520 μs that was comparable to that measured in the other pores.

Since it was not affected by the dilute concentration of protein, the extent of the blockade distribution was not attributed to multiple molecules competing for the same pore. However, the distribution was affected by the denaturation conditions (e.g. if the exposure to heat was too short, i.e. ≤30 min) To account for the differences in the duration and fractional blockades, it was assumed that each protein translocation explored different aspects of the pore topography through: 1. different alignments of a rigid, rod-like protein relative to an irregular pore with a sub-nanometer-cross-section; and 2. different trajectories for hydrated ions moving through a blockaded pore of comparable size. Thus, the blockade distribution was attributed to factors relating to conformational noise such as a persistent, native-like topology in the denatured protein unraveling in the pore,(33) or the initial configuration of the molecular termini relative to the pore, or different orientations (N-terminus versus C-terminus or yaw/twist about the vertical axis) of the rigid, rod-like molecule relative to the pore topography.

Taken together, these observations support the assertion that ΔI/I₀ was a measure of the molecular volume occluding the pore. It was reasoned that the sensitivity of a sub-nanopore to changes in the molecular volume would lead to chemical specificity that could be used to discriminate between proteins and possibly even amino acid residues based on their volume. Following up on this notion, two pure denatured protein solutions, one containing 300 pM denatured CXCL1 and another 300 pM denatured BSA, were analyzed by stochastic sensing⁽⁶⁶⁾ using a pore with a 1.4×1.6 nm² cross-section using an applied bias of 1V to drive molecules through the pore. The compiled blockade current distributions were evidently multivariate. The aggregate distributions were represented by normalized heat maps of the probability density functions (PDF) reflecting the number and distribution of events (FIG. 5h,i). A contour representing the PDF from CCL5 (FIG. 5f) was juxtaposed on the PDF heat map corresponding to BSA (FIG. 5i) to illustrate that the PDFs are distinct. The point-by-point difference between the PDFs, i.e. (PDFₚᵣₒₜᵢₑₙ₁−PDFₚᵣₒₜₑᵢₙ₂)² revealed dissimilarities. By integrating these differences over the entire blockade current space, a metric of the statistical distance between the two PDFs was obtained—namely, Δ, which is related to the energy distance and also the Cramer's distance.⁽⁶⁷⁾ The energy distance between the PDFs representing the BSA and CCL5 measured with the same pore was a Δ=1.6×10⁻⁴, indicating very different distributions. In contrast, two peptides (such as H3N and H3A) that differ only by a single chemical modification to a single residue were ostensibly indiscernible, even with sub-nanopore. Specifically, two pure denatured protein solutions, one containing 250 pM H3N and another 250 pM H3A, were analyzed separately using the same pore with a 0.5×0.6 nm² cross-section at a 0.7 V bias to find that Δ=3.9×10⁻⁵, which shows the similarity between the two peptides (FIG. 5j,k). These simple tests indicated that stochastic sensing could discriminate proteins, but it was not specific enough to differentiate between species that were very similar, which differ by a single post-translational modification, for example.

Strikingly, scrutiny of fluctuations observed within each blockade exposed signatures of the protein sequence. Each blockade within a subset of the distribution from ΔI/I₀>0.30 for 1<Δt<70 ms comprising the majority of blockades, revealed nearly regular fluctuations beyond the noise level, the number of which corresponded closely to the number residues in each type of protein (FIGS. 6a-d and FIG. 6e). The Fourier amplitude associated with the fluctuations varied by more than an order of magnitude, and it was frequently more than four standard deviations beyond the noise found in a trace of the open pore current of comparable duration. In particular, a tally of the number of fluctuations in blockades associated with translocations of denatured CCL5 yielded N_{CCL5}=65.0±3.3 regardless of the duration of the blockade (FIG. 6e), which coincided with the 68 AA residues in the mature protein. Likewise, a protein with a similar length, CXCL1, tallied a similar number of fluctuations, N_{CXCL1}=62.6±9.3, corresponding to the 71 residues in the protein. In contrast, a much larger number of fluctuations, N_{BSA}=602.0±64, was observed under the same conditions when denatured BSA blockaded the pore, which agreed within the error with 583 AAs in the mature protein. Finally, far fewer fluctuations, N_{H3N}=20.5±1.3, were observed when denatured H3N (or the related conjugates) was impelled through a sub-nanopore, corresponding to a peptide with 21 residues.

Although Fourier analysis was the primary means for counting the number of fluctuations in a blockade, another method that used a Gaussian fit to the peaks to identify and tally them gave similar results for each type of protein. For example, for the traces depicted in FIG. 6a-d, the counts (orange circles) were N_{CCL5}=67, N_{CXCL1}=69, N_{BSA}=593, and N_{H3N}=21. When the same algorithm settings were applied generally, this approach yielded on average N_{CCL5}=60±29 peaks for CCL5, N_{CXCL1}=62±26 for CXCL1, and N_{H3N}=22±12 peaks for H3, which are all consistent with the number of AAs in the mature proteins within the error. BSA required changes to the settings, but still yielded N_{BSA}=601±152 peaks in agreement with the residues constituting the mature protein. Since fluctuations like these were not observed in pores with cross-sections >(0.7±0.1)×(0.8±0.1) nm² or in the absence of SDS, it was inferred that the pore topography and the denaturation agents SDS and BME were causative factors. However, whereas the fractional blockade improved, when the pore diameter was reduced to a nominal value of 0.3 nm, the amplitude of the fluctuations relative to the noise did not. Thus, the smallest volume pores under the conditions tested here were not optimal for detecting fluctuations.

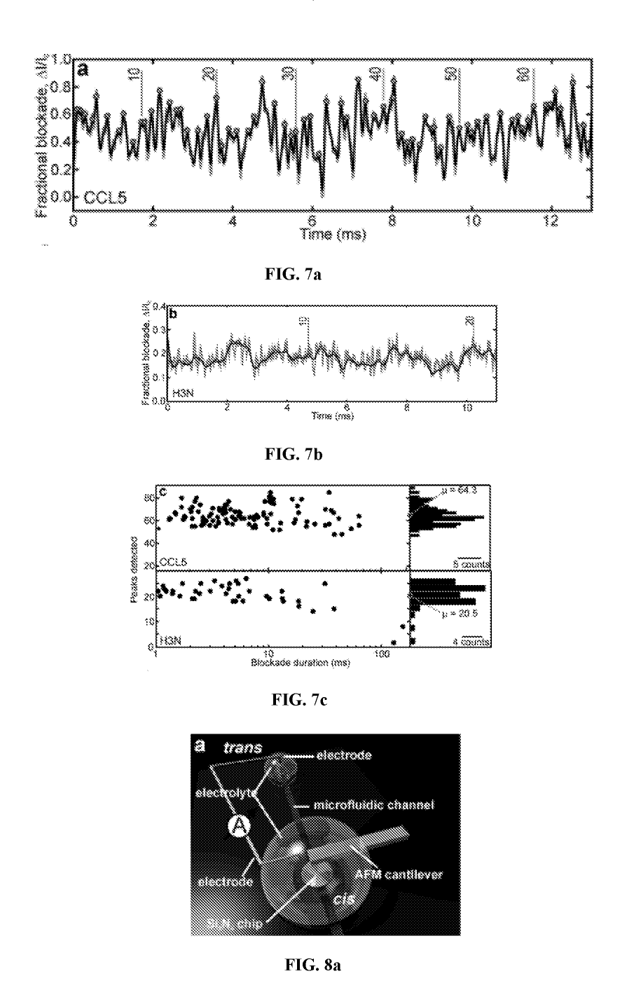

Because of the correspondence between the number of fluctuations and AA residues in the protein, it was asserted that each fluctuation reflected a read of the AA sequence of a single protein molecule. Since all the blockades were recorded using the same bandwidth, the observation that both the number of fluctuations and the patterns within a blockade persisted over a range of durations and fractional currents for most of the blockades (FIGS. 7a,b), precludes random noise as the sole explanation for the fluctuations. The analysis of the fluctuation patterns indicated two distinct groups, which showed similar peak maxima under temporal inversion (FIG. 7c). This observation was interpreted as evidence of two nearly equivalent but opposite translocation directions—either N-terminus or C-terminus first. Therefore, all the blockades were sorted into two groups and the second was inverted in (normalized) time, depending on the relative correlation of their observed peaks. (72% of CCL5 and 69% of CXCL1 events remained unflipped, indicating a preferential translocation direction, whereas 54% of BSA and 62% of the H3N events remained unflipped.) A simple binomial t-test, assuming a null of 50% of events flipped for random noise, indicated that the number of flipped events observed was p<10⁻⁶ for all proteins, given the sample sizes used. Therefore, the data consistently showed a preferential direction for the translocation (N terminus first). Other discrepancies between the patterns observed in the majority of blockades were attributed to misreads such as a skip or multiple reads of the same AA. Misreads give rise to a different tally of fluctuations or gross irregularities, which may be associated conformational noise. For example, lags were also observed in the fluctuation pattern (FIG. 7d). These accounted for 5 to 25% of the blockades, depending on the bias and the protein), and were ascribed to time-consuming reconfiguration of the molecular termini just prior to insertion into the pore. Lags were culled from the distribution. Finally, multilevel events were also observed, especially if the concentration of SDS was <0.0001% (1-10 μM for 100-300 pM of protein), but only rarely, accounting for <5% of blockades typically, which may be associated with the protein unfolding in the pore (FIG. 7e). These were also culled.

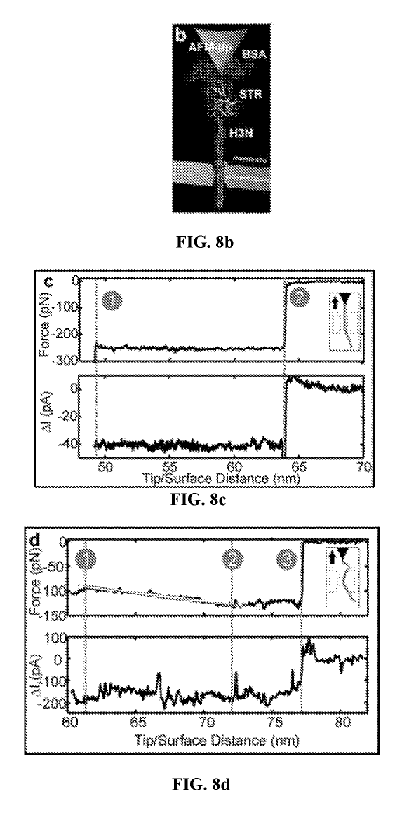

The amplitudes of the fluctuations observed in different blockades (of varying duration and fractional blockade current) from the same protein were highly correlated to each other. This assertion was rigorously tested by comparing the fluctuations in each blockade. Since neither the duration or the fractional current were perfectly uniform, each blockade was normalized in time and the average fractional current was zeroed for comparison (FIGS. 7a-c, and 5 top, blue lines). A consensus was then formed from the average of a number of blockades (red lines)—each associated with the translocation of a single molecule. The mean Pearson product-moment correlation coefficient between the consensus and an individual blockade was 0.42 for CCL5 (FIG. 8a), 0.55 for CXCL1 (FIG. 7b), 0.67 for H3N (FIGS. 7c) and 0.23 for BSA (FIG. 5) Thus, the fluctuations persisted even after averaging, unlike the open pore current noise (FIG. 6).

Since the fluctuations were correlated and nearly regular, it was inferred that the SDS must impart a nearly uniform negative charge density along the polypeptide resulting in a consistent electric force working to impel the molecule through the pore. It is likely that counter-ions moved along with a protein molecule decorated by SDS to minimize Coulomb repulsion, but since a pore has a diameter comparable to a hydrated ion, the motion of the ensemble through it would be impeded by steric hindrances. Thus, the coincidence between the number of fluctuations and AAs in the protein, and the near-regularity of the patterns were consistent with a tightly choreographed, turnstile motion of AAs through the pore in which a single AA stalls repeatedly in a well-defined conformation within the pore and then eventually proceeds through the pore due to the electric force on the molecule. This type of motion has also been observed when single stranded DNA is forced through a 1-nm-diameter pore.⁽⁶⁸⁾

If each fluctuation within a blockade reflects an event in which one AA enters the pore while another leaves, then it was reasoned that the amplitude of the fluctuation should be attributed to the occluded volume associated with the AA residues in the pore. Because the pore current was crowded and most of the potential dropped near the waist, then each fluctuation would measure the occluded volume there due to 3-5 AAs, with the exception of the first and last fluctuations at the inception and termination of a blockade. These were interpreted as a reduced sum of AAs, i.e. <3-5. Consistent with this reasoning, consensuses formed by averaging together normalized and binned blockades were found to be highly correlated to models for the peptide chains in which each residue was represented by its volume. The volumes used in the models depended on the empirical approach used to estimate them and each has attendant uncertainties (e.g. some are more affected by hydration), but all the models based on different types of volume measurements were all highly correlated with each other (0.96-1.00).⁽⁶⁹⁾ Taking estimates obtained from crystallography data, the primary structure of the protein was translated into a sequence of AA volumes. To account for the current crowding in the pore waist as the molecule steps successively, one AA at a time, through the peptide chain, a moving average with a window size ranging from k=3 to 5 AA volumes (depending on the pore topography) was performed on the sequence of volumes.

The models based on AA volumes were found to be correlated to the empirical consensuses, and the agreement improved as the number of blockades included in the consensuses increased. For example, error maps were produced by partitioning 400 CCL5 blockades into seventeen consensuses (FIG. 7a, bottom), each of which was then compared to the model. The agreement for each read was expressed as a percentage, and subsequently identified as either a correct (gray) or incorrect (black) call, depending on whether the agreement was greater or less than 20%, respectively. In this way, the seventeen consensuses for CCL5 exhibited an average percentage read accuracy of 59.4%. In contrast, the entire 400-element consensus produced a mean percentage read accuracy of 65.2% for the same 20% threshold tolerance. Therefore, increasing the number of blockades in the consensus improved the agreement with the model. For CCL5, the consensus correlation to a k=3 model was 0.75. Likewise for CXCL1, the correlation of the k=5 model (for a pore with a longer waist) with the 45-blockade consensus was 0.51, with an associated mean percentage read accuracy of 84.7% for a 20% threshold tolerance, and 50% for a more stringent 10% threshold on the triplet volume measurement. The performance on the shortest peptide, H3N, was similar. The correlation of a k=3 model with a single event was 68%, but a 52-blockade consensus showed only two positions out of 21 outside the 20% threshold tolerance—a read accuracy of 90%. All of these compare favorably to BSA (Fig. S5) for which the correlation of the k=5 model with the 41-blockade consensus was 0.35, with an associated mean percentage read accuracy of 68.4% for a 20% threshold tolerance, and 38.9% for a more stringent 10% threshold on the triplet volume measurement, which is nearly double the random noise at this threshold (FIG. 6). The lower correlation for BSA likely reflects more misreads, i.e. skips and repeats.

There are several qualifications required on the read accuracy. First, due to the current crowding at the pore waist, each read likely reflects the occluded volumes associated with multiple AA residues. Thus, the number of correct reads obtained for each protein (CCL5: 65.2%, BSA: 68.4% CXCL1: 84.7%, H3: 90%) does not reflect the accuracy with which single residues can be called. The threshold for a correct read was chosen to be 20%, which means that, on average, ±20% of optimally ranged and fitted random noise would fit the model because 40% of all data will fall within its threshold boundary. So, to what extent is the read accuracy (77% on average) statistically significant? To establish a null dataset for comparison to this value, regions of open pore current recorded from pure electrolyte (250 mM NaCl) were sampled with a distribution of blockade durations reflecting the measured distributions. These false events were then optimally flipped, ranged and fitted to the model for CCL5 and their read accuracies were found to have a Gaussian distribution with mean of μ=38.6% and a standard deviation of σ=5% for 20 runs. According to this mean and standard deviation, the true read accuracies found for each protein were more than 6σ occurrences with respect to the noise. Thus, from this significance level, each fluctuation represents a low fidelity read that measures the occluded volume associated with 3-5 AA residues in the waist of the pore.

Low fidelity reads with multiple monomers affecting the blockade current like this should not pose a problem for sequencing protein, provided that the translocation rate is controlled and the coverage is high. Although it is cumbersome to accommodate protein, Viterbi algorithms⁽⁷⁰⁾ or L1-penalized logistic regression⁽⁷¹⁾ might be adapted to decode the sequence from these reads with single AA resolution. However, regardless of the coverage, read fidelity could still be compromised by systemic errors. A further analysis of the read fidelity and cross-correlation between proteins exposed several interesting trends. For example, it was apparent from the error maps (FIGS. 7a-c, S5, bottom) that the correlations to the model do not accumulate randomly; e.g. discrepancies were consistently found near positions 1, 11, 19, 21, 31, 53, 58 for CCL5, and positions 6, 12, 29 and 30 for CXCL1 and positions 11, 12 for H3N (although these were suppressed in the consensus). By assigning errors to the a-priori sequence of cumulative AA volumes present at each position, the volume error for each AA was calculated (FIGS. 7d-g; FIGS. 5b,c) that, taken together, indicated the source of the errors. In particular, negatively charged AAs (D, E) repeatedly showed the highest read errors for these three proteins. On the other hand, the two (positively charged) lysines (K) at position 54-55 both exhibited a mean read accuracy of 92% for the seventeen separate runs for CCL5 (FIG. 7a, bottom). Finally, AAs with small volumes (A, C, G, and S) were frequently misread, which can be rationalized because the volume associated with the error (0.025 nm³) is only <10% of the effective pore volume.

Thus, it was possible to discriminate proteins by reading the sequence of AA residues through measurements of fluctuations in the blockade current. However, the relative insensitivity to AA residues with small volume prompted the question: Short homopolymer constructs are not good candidates to test the sensitivity of a pore with a sub-nanometer cross-section because homopolymeric amino acid tracts are involved in protein-protein interactions and have intrinsic polymerization properties that might confound the interpretation of the blockade current.⁽⁷²⁾ Instead of homopolymers, the sensitivity of a sub-nanopore was tested using post-translational modifications of specific residues. Post-translational modifications (PTMs) such as acetylation, methylation, and phosphorylation introduce new functional groups into the peptide chain that extend protein chemistry beyond the twenty-two proteinogenic AAs. To measure the sensitivity to PTMs, the three variants of the tail histone H3, H3N, H3A, and H3M, were analyzed and compared. The epigenetic control of chromatin structures have been linked to the covalent modifications of histone tails like these; H3A is an activated promoter, while H3M is a repressor.^{(72, 73)} However, these modifications were especially interesting in the context of a blockade current measurement because the changes associated with the occluded volume were expected to be like that associated with glycine due to the similarities in molecular weight. For comparison, three consensuses were formed: one associated with 304 blockades of H3; and another with 231 blockades from H3A and a third with 958 blockades from H3M (FIGS. 8a,b, top), each was acquired from nominally 0.5-nm-diameter pores at 0.7 V using protein concentrations of 250 pM in 250 mM NaCl. Subsequently, the consensuses for the conjugates were optimally fit and ranged to the mean fractional blockade of the native protein.

The juxtaposition of consensuses clearly showed the positional sensitivity of the fractional blockade current (FIGS. 8a,b, top). The fractional blockade associated with both chemical modifications was enhanced between read positions 6 and 11. In addition, a prominent feature was observed near read position 9 in H3A measured relative to H3N, in correspondence with the expected change on K9 due to acetylation. In contrast, a depression appears near read position K9 in H3M measured relative to H3N. Fitting the difference in the fractional blockade between the chemically modified and native traces to a simple top-hat form revealed differences beyond the noise over a range of 3.9 positions (FIG. 8a, bottom), which corresponds closely to the FES estimate obtained for the number of AAs in the waist and substantiates the claim that each read reflects about four AAs. Likewise, the difference in the fractional blockade between the H3M and H3N traces extends over a range of 4.2 read positions (FIG. 8b, bottom). Therefore, based on the sensitivity to single modifications of single residues, the fluctuations in the fractional blockade current are a measure of the moving average of the occluded volumes associated with a quadromer (about four AAs residues).

A new method for the detection of AA quadromers in the sequence of a protein molecule that uses a sub-nanopore through a thin inorganic membrane was demonstrated in the present example. When a protein, denatured by heat, SDS and BME, was impelled by an applied electric field through a sub-nanopore, nearly regular current fluctuations were observed in a majority of current blockades that coincided with the number of residues in the protein, regardless of the duration or fractional blockade current. The amplitudes of the fluctuations were highly correlated with the volume occluded by quadromers (four AAs) in the protein sequence located in the waist of the pore near the center of the membrane. Moreover, if the consensus was large enough, this method was sensitive enough to detect chemical modifications to a single residue. Thus, this method can be used to discriminate proteins with a similar number of AA residues that differ by post-translational modifications. If each fluctuation represented a read of the quadromer, then the read fidelity was low, but it is still more than double the accuracy of electrical noise. With sufficient coverage, this methodology might be useful for sequencing protein if dynamic programming algorithms can be adapted to untangle the sequence with single AA resolution. The extreme sensitivity and the prospects for long reads with a sub-nanopore offer compelling advantages that, with further development, may someday transform molecular diagnostics. However, initially this method is likely to be used to augment the short reads offered by techniques such as mass spectrometry with long reads for aligning and quantitation.

Methods: Sub-nanopore Fabrication. Pores with sub-nanometer cross-sections were sputtered through thin silicon nitride membranes using a tightly focused, high-energy electron beam carrying a current ranging from 300-500 pA (post-alignment) in a scanning transmission electron microscope (STEM, FEI Titan 80-300, Hilldooro, Oreg.) with a Super-TWIN pole piece and a convergence angle of 10 mrad. For example, a 398 pA beam was used to sputter a nominally 0.3-nm-diameter pore in 50 sec. The silicon nitride was deposited by LPCVD directly on the top surface of a polished silicon handle wafer and a membrane was revealed using an EDP (an aqueous solution of ethylene diamine and pyrocatechol) chemical etch through a window on the polished back-side of the handle. The thickness of the membranes, which ranged from t=8 to 12 nm, was measured in situ using electron energy loss spectroscopy (EELS) prior to sputtering. The roughness of the membrane, measured with custom-built silicon cantilevers (Bruker, Fremont, Calif.) with 2 nm radius tips, was estimated to be <0.5 nm-rms.

Multi-slice Image Simulations. The details of this methodology is provided in Example 1.

Microfluidics of this procedure are provided in Example 1.

Low-noise electrical measurements: To perform current measurements, a sub-nanopore was immersed in 200-300 mM NaCl electrolyte and allowed to stand for 1 da (typically) to wet the pore, a transmembrane voltage was applied using Ag/AgCl electrodes and the corresponding pore current was measured at 22±0.1° C. using an Axopatch 200B amplifier with the output digitized with DigiData 1440 data acquisition system (DAQ, Molecular Devices, Sunnyvale, Calif.) at a sampling rate of 100-250 kHz. Clampex 10.2 (Molecular Devices, Sunnyvale, Calif.) software was used for data acquisition and analysis.

To measure a blockade current, a bias ranging from −0.3 V to −1 V was applied to the reservoir (containing 75 μL of electrolytic solution and 75 μL of 2× concentrated solution of protein and denaturant) relative to ground in the channel. The background noise level was typically 12 pA-rms in 250 mM NaCl solution at −0.7 V. Recombinant, carrier-free protein was reconstituted according to the protocols offered by the manufacturer (R& D Systems). Typically, the protein was reconstituted at high (100 μg/ml) concentration in PBS without adding BSA to avoid false readings. From this solution, aliquots diluted to 2× the concentration of denaturant with 200-500 pM protein, 20-100 μM BME, 400 mM NaCl with 2-5×10⁻³% SDS were vortexed and heated to 75° C. for 15-60 min. The solution was allowed to cool and added in 1:1 proportion with the (75 μL) electrolyte in the reservoir and allowed to sit for >30 min. Data was recorded in 3 minute-long acquisition windows. If a pore became clogged with protein, the data set was cropped using Clampfit (Axon). When a clog occurred, both the channel and reservoir were flushed with 18 MΩ de-ionized water for at least 5 min. to clear the pore. If this procedure failed to clear it, although failure of the pore was usually indicated, 0.1% SDS solution was flushed through the channel to disperse latent aggregated protein in an attempt to recover the pore. In this way, sequencing data was acquired from one sub-nanopore for >28 da.

Signal estimation. Data handling involved five steps: 1. selection of events of sufficient duration from the raw current trace; 2. fitting of fluctuations within events to peaks; 3. rescaling of events in time to the same number of datapoints and current level for averaging; 4. alignment of event translocation directions; and 5. renormalization of the consensus traces for comparison to the model occluded volume.

1. Blockades were initially extracted from current traces recorded with a 10 kHz eight-pole Bessel filter using OpenNanopore—but not always reliably,⁽⁷⁸⁾ and so we resorted to custom MATLAB code. These codes allowed for the manual removal of multilevel events and open pore regions incorrectly categorized as true events. The settings for OpenNanopore were optimized by manual inspection of the open pore noise and the blockades. The magnitude of the blockade, ΔI, local open pore current, I₀ and blockade duration, Δt, were calculated for each event. Events with sufficient duration to detect single AAs (assuming linear velocity) were selected according to the average number of intra-event peaks observed for a given protein, C, and the acquisition bandwidth cutoff, D, i.e. τ>2C/D. Within this subset, blockades exhibiting a mean amplitude that was both five standard deviations (5σ) above the noise and within 10% of the mean expected percent blockade were selected.

2. Custom MATLAB code was written in order to interrogate events for an initial number of fluctuations. Fourier analysis of the event allowed for the detection of peak frequencies within an event, which were then compared to the peaks present in equivalently long duration regions of open pore current (FIG. 8). Within a broad window, covering at minimum ±50% of the number of peaks expected (e.g. 300-900 peaks for BSA), the maximum peak difference between the open pore and a blockade was determined. This value (in Hz) was then converted to an estimated number of peaks given the event duration. The average number of peaks observed for a given protein was typically less than 10% from the known number of AAs for each protein.

To validate these results, a custom algorithm was develop in MATLAB to automatically count peaks. The algorithm worked by fitting the data to an array of Gaussian peaks. First, all the local maxima were identified and categorized depending on whether or not they conformed to a Gaussian peak profile. Peak positions not close to a Gaussian maxima were discarded. Second, if two or more peaks were too close together (assigned within the boundary of the same Gaussian), all but one was discarded. The average number of peaks was then determined from all events for each protein. However, it was observed that the number of fluctuations found for this approach scaled with the event duration due to the increased number of noise-related peaks in longer dwell-time events, which gave rise to the large range of the values obtained, evident in their larger standard deviations. For visualization of the fluctuations, events were smoothed using a smoothing spline algorithm included in the MATLAB Curve Fitting Toolbox. The stiffness of the spline was adjusted until the number of fluctuations in the smoothed event equaled the number of AAs in the protein.

3. Assuming the peaks were periodic in time, all events were linearly re-sampled either into N bins, where N is the average number of peaks observed per event or 10,000 data points. For the purpose of averaging, all events were scaled to contribute equally to the final consensus traces.

4. Before averaging was performed, it was noted that blockades comprised two distinct groups, which showed similar peak maxima under temporal inversion. This observation was interpreted as evidence of two equivalent translocation directions and so all events were sorted into two groups and the second was inverted in (normalized) time. The event blockades were then renormalized according to the median blockade percent and averaged.

5. The model developed for occluded volume shows variations in nm as a function of position. However, the events were recorded in units of pA. The scaling of pA to nm³ was necessary to compare the model and the consensus events, and can be directly inferred using both the pore geometry and open pore current. However, the ordinate of events was lineally scaled to the volume model using a Nelder-Mead method search.

Contours, maps and error assignments. Contours were created according to the density of data points in logarithmic duration-fractional blockade-space, based on a kernel density function whereby every data point contributes a 2D Gaussian to the cumulative contour, which was then normalized in ‘z’ such that the entire volume of all contributing data integrated to one. The measure of energy distance for two such contours was calculated from the net sum of the squared differences between the two normalized density functions.

Error maps (FIG. 7) were used to show regions of agreement and disagreement between the model (V) and a consensus (C) as a function of read position. Regions of error were coded with different gray-tones. If the error was greater than a given threshold, E=|(C_{1 . . . n}−V_{1 . . . n})|/V_{1 . . . n}≥T, it was indicated as black and elsewhere it was indicated as gray when considered consistent with the model. Similarly, errors as a function of read position were found by contributing the vector of errors (as described above) at each site to all possible AAs recorded at that read position. For example, consider a single event where an error of 6% on read position 5 could have aris3n from any part of the position trimer {4, 5, 6}. After these assignments, all possible errors on every AA were then summed and normalized to the total observed error on the event and plotted as a function of AA and AA volume for each protein.

Finite Element Simulations: Finite element simulations (FESs) of vacated (open) pores, which ignored the atomistic details of the structure and electrolyte, were used to examine the distribution of the electrostatic potential and current (Fig. S2). FESs of the electric field and the electro-osmotic flow were performed using COM SOL (v4.2a, COM SOL Inc., Palo Alto, Calif.), following a Poisson-Boltzmann formalism described elsewhere.⁽⁷⁹⁾ Briefly, the applied potential ϕ and the potential Ψ due to charges in the pore are decoupled from one another and solved independently. The relationship between ϕ and the charge carriers, Na+ and Cl−, is given by the Poisson equation, ∇ϕ=−ρ/εε₀, where, and ρ, ε, and ε₀ are the volume charge density, and the relative and vacuum permittivities respectively. The charge density is given by ρ=FΣZᵢCᵢ where F=96,485 C/mol is the Faraday constant, zᵢ is the valence and cᵢ is the molar concentrations of ionic species i. The distribution of ions close to charged surfaces satisfies the Boltzmann distribution; thus, the charge density is given by cᵢ=c_{o,i} exp)−zᵢeΨ/k_{B}T), where c₀ is the molar concentration far from the sub-nanopore (i.e. bulk concentration), e is the electric charge, k_{B}=1.38×10⁻²³ J/K is the Boltzmann constant, and T=298° K is the temperature.

Electro-osmotic flow is expressed by the Navier-Stokes equation, η∇²u−∇p−FΣᵢzᵢcᵢ∇V=0, where V=ϕ+ϕ,η is the viscosity, p is the pressure, and u is the velocity. The transport of ionic species is described by the Nernst-Planck equation given by Dᵢ∇²cᵢ+zᵢuᵢcᵢ∇²V=u·∇V cᵢ where Dᵢ is the diffusion coefficient and μᵢ is the ionic [symbol] i^{th} species. In this treatment, u, V and cᵢ are coupled between equations. The relationship between the surface charges σₛ and the zeta potential ζ is given by the Grahame equation:⁽⁸⁰⁾ σₛ(ζ)=√8c₀εε₀k_{B}T sihnh(eζ/2 k_{B}R). The boundary conditions for the system are given in Table 1.

### Example 3—Sequencing Antibodies with a Synthetic Sub-Nanometer Pore Inorganic Membrane

The specificity of an antibody for an antigen is exquisitely sensitive to the antibody amino acid (AA) sequence, post-translational modifications (PTMs) of it and rearrangements of the structure. Whereas high-throughput DNA sequencing is routinely used to indirectly inform on the primary structure and diversity of antibodies,^{(81, 82)} PTMs and structural rearrangements (as occurs with 1gG4) require direct analysis of the protein itself:⁽⁸³⁾ The methods used prevalently for sequencing protein directly, mass spectrometry (MS) and Edman degradation (ED), suffer limitations associated with short reads and demand concentrated samples.

This example provides a tool that uses a sub-nanometer diameter pore through a thin, charged membrane to directly sequence the AAs and PTMs in a single antibody molecule by measuring fluctuations in the blockade current when the molecule is impelled through the pore. The sequence from whole antibodies, derived from hybridoma supernatant:⁽⁸⁴⁾ will be read through measurements of the blockade current and decoded using large-scale dynamic programming (DP). Concurrently, using DNA/RNA extracted from the same hybridomas, the sequence will be validated with 3^{rd}-generation sequencing.

The tool provides a method by which proteomics for amino acid containing molecules is broadly provided, and thus provides for the transition of molecular diagnostics from the lab into the clinic.

A sub-nanopore offers the prospect of de novo sequencing of a single whole antibody. This is not evolutionary, but disruptive technology. Nanopores have been used to detect and analyze polypeptides and proteins before, but not for sequencing, primarily because: 1. the secondary structure of a protein confounds the interpretation of a current blockade; 2. the translocation kinetics are out of control; and 3. nanopores lack the chemical specificity—detection of an AA even in a pore 1-nm-diameter is unfeasible.⁽⁸⁹⁻¹⁰⁰⁾ These difficulties are overcome with the present methodologies and materials. AA quadromers in the sequence of a denatured protein molecule are shown here to be discriminated using a sub-nanopore. When a sub-nanopore was immersed in electrolyte and a voltage was applied across it, measurements of a blockade in the current through the pore, associated with the translocation of a denatured protein molecule, revealed nearly regular fluctuations, regardless of the blockade duration. The number of fluctuations coincided with the number of residues in the protein and the amplitude of each fluctuation was highly correlated with the volume occluded by quadromers (four AA residues) in the protein sequence. Additionally, close examination of the fluctuation amplitudes revealed that a sub-nanopore was sensitive enough to detect the occluded volume associated with PTMs at a single AA within a quadromer. Thus, each fluctuation in the blockade current represented a read of a quadromer in a single protein molecule. By using DP algorithms (such as Viterbi), the sequence of AAs can be decoded from these fluctuations in the blockade current.

Sequencing protein with a sub-nanopore will reveal not only the primary structure, but also the structural rearrangements, mis-translations and PTMs that account for the phenomenal diversity of the antibody repertoire. Data collected here on the force and blockade current, characterizing the translocation of a single protein molecule tethered to the tip of an atomic force microscope (AFM) cantilever as it was impelled systematically (4 nm/s) through a sub-nanopore, revealed a dichotomy in the translocation kinetics: either the protein slid nearly frictionlessly through the pore or it slipped-and-stuck to the membrane. When the molecule translocated frictionlessly, periodic fluctuations were observed in the force and blockade current with lags that corresponded to the separation between AAs, and with amplitudes (in the current) that correlated with the occluded volume of quadromers in the protein sequence as observed when the molecule was untethered. With the molecule tethered to the tip, the read accuracy improved, likely due to the systematic and slower frictionless translocation kinetics. On the other hand, slip-stick kinetics confounded the interpretation of the blockade current and so, control of the translocation kinetics is essential for sequencing.

The methods presented here for the reading of an amino acid sequence of an antibody with a sub-nanopore demonstrate the following advantages, among others: 1. precision over the pore topography make it smaller than the secondary structure of a protein with a cross-section near the waist comparable in size to a hydrated ion, and also enhances the chemical specificity; 2. the use of charge and an integrated electrode on the membrane to balance the hydrophobicity and electrostatics produces frictionless translocation kinetics; 3. the large-scale DP used to decode the sequence from the fluctuations measured in the current blockades; and 4. validation of the protein structure through the use of 3^{rd} generation sequencing of DNA/RNA coupled with a solution-phase capture methodology to directly correlate the entire antibody transcript to the protein sequence.

Antibodies are used by the immune system to identify and neutralize pathogens and so, as both diagnostic and therapeutic agents, they are indispensable to medicine. By facilitating extensive, inexpensive protein sequencing, the present methods that employ a sub-nanopore thin inorganic membrane methodology provides a method for identifying and creating new naturally occurring and synthetic antibodies. Secreted by B-cells in the adaptive immune system, antibodies are used to identify and neutralize pathogens. The paratope of an antibody binds specifically to the epitope on an antigen, tagging it as a foreign pathogen for attack by the immune system or neutralizing it by inhibiting some facet essential for infection. The specificity of an antibody is exquisitely sensitive to the AA sequence, PTMs and rearrangements of the structure, but the structure of the whole antibody plays a role.

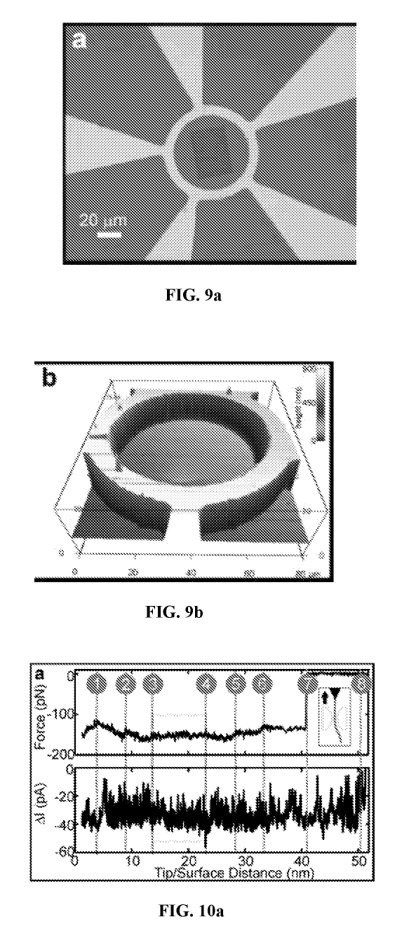

Human 1gG antibodies are large Y-shaped glycoproteins, consisting of two heavy (450-500 AAs) and two light (211-217 AAs) polypeptide chains linked by intra- and inter-chain disulfide bonds (FIG. 1). Each chain is divided into two domains, the variable (V) and constant (C) regions. Complementarity-determining-regions (CDR) that comprise the V-regions form the antigen-binding sites. The fork in the Y is the hinge region, ranging from 12-62 AAs long. Whereas the primary AA sequences of the C-regions of the heavy-chains are greater than 95% homologous between different antibodies, major structural differences, such as the number of residues, are found in the hinge—it is the most diverse structural feature differentiating 1gGs. The hinge links the two Fab arms (top) to the Fc stem (bottom), allowing them to swing and interact with incommensurately spaced epitopes and adopt different conformations. The principal determinate of the specificity is the length and sequence of the CDR-H3 region (FIG. 9) in the heavy-chain of the antibody, but specificity can also be dictated solely by the light-chain.⁽⁸¹⁾ The number of patterns that may be generated for the paratope outnumber the number of cells in a human body (>10⁽⁹³⁾). Most of the diversity in the antibody repertoire evolves from V(D)J recombination, which involves the recombination of a set of variable (V), diversity (D) and joining (J) gene segments from the germ line.

Whereas DNA/RNA sequencing is routinely used to indirectly characterize the primary structure,^{(81, 82)} protein structural rearrangements and PTMs can only be revealed by direct protein-level analysis of the whole antibody.⁽⁸³⁾ For example, IgG4 undergoes a half-antibody exchange in vivo in which the heavy and light chains in the Fab arm are swapped with another molecule resulting in a recombined antibody composed of two different binding specificities—breaking all of the rules that immunologists take for granted.⁽¹⁰⁴⁾ De novo protein sequencing of antibodies can capture these aspects of the structure, and it is also essential when the original cell line is not available, or to assess the scope of polyclonal response. Sequencing antibodies is a formidable task, however. The two methods used prevalently for sequencing protein, MS and ED, suffer limitations associated with short reads. On the one hand, with short (less than 30 residues long) peptide reads, ED has low throughput requiring proteolytic digestion and peptide fractionation. On the other hand, MS can sequence a protein of any size, but it relies on enzymatic digestion so it becomes computationally demanding to reassemble the fragmented sequence as the size increases. In addition, MS requires concentrated samples (>fmole/L-scale) and the machine has a foot-print the size of a room. To remedy these deficiencies, the present technologies may be implemented to remedy the prior limitations associated with long reads of a polypeptide. With the present single molecule sequencing, which extracts the maximum amount of information from minimal material, the development of improved protein sequencing technology may be realized.

A sub-nanopore offers the prospect of de novo sequencing of single protein molecules. Nanopores have not been used for sequencing peptides and proteins before. The secondary and higher order structure of the protein confounds the interpretation of the blockade current and overwhelms the chemical specificity. To recover the signal-to-noise ratio (SNR) required for sequencing, a methodology such as the ones disclosed herein with smaller pores (sub-nanopores) and denatured protein is provided in the present techniques. Moreover, the charge distribution along the native protein is not uniform, which frustrates the systematic control of the translocation kinetics by the electric field in the pore.⁽¹⁰⁰⁾ Instead, in conjunction with a field, enzymatic motors are used to drive amino acids stochastically through the smaller pores by repeatedly pulling on individual amino acids.

Nanopore sequencing of DNA reveals advantages and disadvantages. Nanopore sequencing of DNA is unique among modern sequencing methods in that it does not require PCR or sequencing-by-synthesis—it sequences single molecules of DNA directly and uses long kilo-base reads to do it.⁽¹¹⁰⁾ However, single nucleotide resolution demands sub-nanometer control over both the molecular configuration in the pore and the translocation kinetics because the equilibrium distance between nucleotides is only 0.35 nm.^{(111, 112)} The biological nanopore, MspA, conjugated with a polymerase (phi29) that steps the DNA through it, has been used to sequence with 4.5 kb long reads, but not with single nucleotide resolution.⁽¹⁰⁷⁾ Instead, quadromers (4 nucleotides) affect the ion current of each blockade level. Similarly, MinION™, commercialized by Oxford Nanopore, uses a motor enzyme, in combination with an electric field, to drive a single DNA molecule through a biological pore to sequence with 8-10 kb long reads in which six nucleotides affect the ion current of each blockade level. Although the fidelity of the reads is low—the Oxford v7.3 chips show only 86% correct per-read average—sufficient coverage and the application of recently developed bioinformatic tools⁽¹⁰⁹⁾ make it a practical DNA sequencer. But the nanopore methodologies used for DNA cannot be easily adapted to protein sequencing: the pore diameter (1 nm) is too large—it lacks chemical specificity—and the chemical denaturation agents would adversely affect a biological nanopore. The present methodologies using denaturing agents for the protein, together with thin inorganic membrane and nanopore and subnanopore surface features, are not suitable for use with a biological nanopore, such as MspA.

To sequence protein with a sub-nanopore, several technical hurdles have to be overcome. First, the protein has to be denatured to eliminate the higher-order structure and facilitate the interpretation of the blockade current.⁽¹¹³⁾ Second, the deficient chemical specificity of a nanopore (related to the volume occluded by the molecule, the charge distribution and the monomer mobility) has to be improved to discriminate AAs.⁽¹¹⁴⁾ If the occluded volume is a measure, then glycine (G) with the smallest volume 0.0664 nm³ has to be detectable. Third, if an electric force field is to be used to systematically drive the molecule through the pore, the charge distribution along the protein has to be uniform, which it is not.⁽¹⁰⁰⁾ Fourth, the translocation kinetics have to be stringently controlled. The data provided herein, characterizing the translocation of a single protein molecule tethered to the tip of an AFM cantilever, has revealed a dichotomy in the translocation kinetics: either the molecule slid nearly frictionlessly through the pore or it slipped-and-stuck to the membrane, but only frictionless slides are consistent with sequencing. According to molecular dynamics simulations (MD), the molecular conformation over the membrane, which is affected by the membrane charge and electrostatics, dictates transport through the pore.⁽¹¹¹⁾ Fifth, if more than one AA affects the blockade current, then the sequence has to be decoded. Since there are 20 proteinogenic AAs, the efficient algorithms used to decode the DNA sequence from blockades cannot be easily co-opted. Finally, the sequence has to be validated with long reads extending beyond the CDR.

The present invention will also provide an instrument that uses a sub-nanopore, through a thin, charged solid-state membrane to sequence AA residues and PTMs in single whole antibodies. The use of a sub-nanopore to directly sequence with long reads of denatured antibodies derived from hybridoma supernatant, using large-scale DP to decode the sequence from measurements of the blockade current; and subsequently validate that sequence with 3^{rd}-generation sequencing of DNA/RNA extracted from the same hybridomas, is also within the scope of uses to which the present technologies will be made. Thus, the sub-nanopore materials and methods provided herein will reveal not only the primary structure of the protein, but also the mis-translations, structural rearrangements and PTMs that produce the diversity of the antibody repertoire.

Translocation Kinetics and Chemical Specificity in a Sub-Nanopore.

Sub-Nanopore Topography:

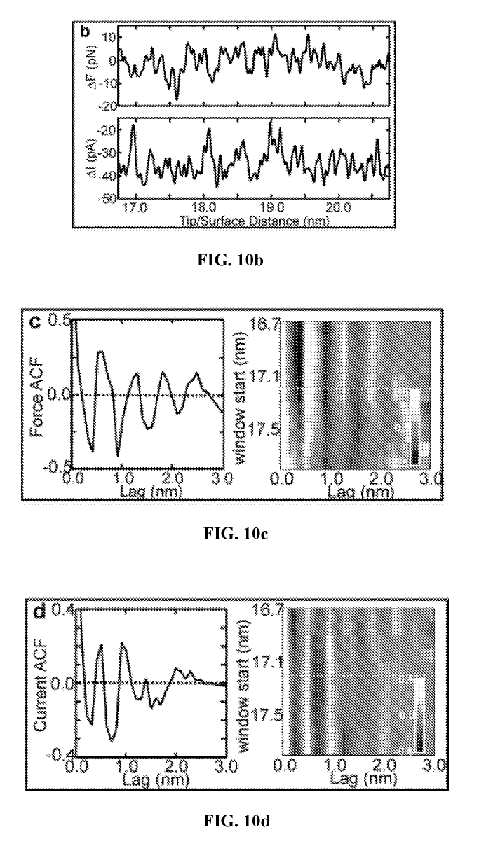

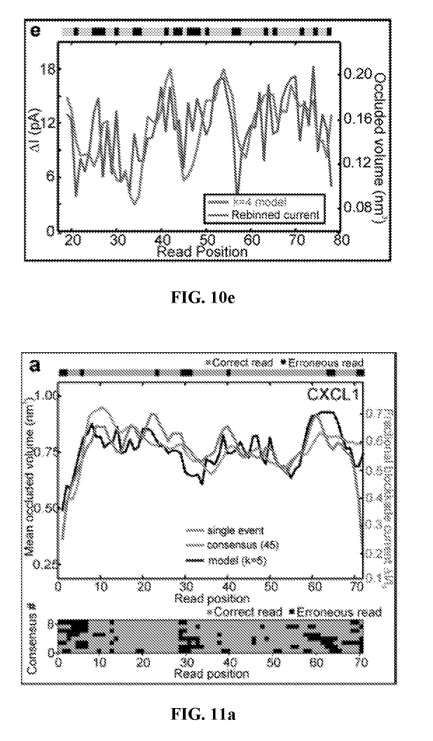

The key to molecular transport through the pore and chemical specificity is control of the electric field distribution in the pore. The precision exercised over the pore topography by electron beam-induced sputtering⁽¹¹⁵⁾ in a scanning transmission electron microscope (STEM) is the linchpin affording us the opportunity to control the electric field (FIG. 10a,i). Sub-nanopores with diameters ranging from 0.3 to 1 nm were sputtered routinely this way. Since the information limit of the microscope was 0.11 nm, to accurately assess the topography, each micrograph was imitated by multislice simulations (FIG. 10a,ii). The simulations reproduced the actual imaging conditions, while accounting for dynamic scattering of the electron beam by the membrane. The close correspondence between the images and simulations signified that the models (FIG. 10a,iii-iv) were realistic representations of the actual pores. From the simulations, it was inferred that the pores were bi-conical, with cone-angles that ranged around θ=15±5°, and irregular. In this example, the cross-section at the waist of (0.4±0.1)×(0.5±0.1) nm². Electrolytic conductance measurements along with finite element simulations (FES) of them provided additional corroborative evidence of the pore size after accounting for pore charge (FIGS. 10b-d). Using the models derived from TEM, FES accurately captured the measured conductances (FIG. 10d). Furthermore, FES revealed that the bi-conical topography crowds the current and focuses the electric field into a region 1.5 nm in extent or k ˜4 AA residues long (FIG. 10c).

The thin inorganic membranes of the present techniques and materials are resistant to high temperature and chemical agents such as sodium dodecyl sulfate (SDS) and β-mercaptoethanol (BME) used for denaturation. SDS is an anionic detergent that works, in combination with heat (45-100° C.) and reducing agents like BME, to impart a nearly uniform negative charge to the protein that stabilizes denaturation. Although the aggregates formed by SDS and proteins have been investigated, the exact structure remains unsolved.⁽¹¹⁶⁻¹²²⁾ Several models have been proposed for it;⁽¹²¹⁾ a “rod-like” model in which SDS forms a shell along the protein backbone was adopted here.⁽¹¹⁶⁾ The resulting uniform charge on the protein facilitated electrical control of the translocation kinetics.

Counting individual Amino Acids: To gauge the electrical signal available for sequencing protein and the electric force required to impel a single molecule through it, the blockade currents through sub-nanopores associated with translocations were measured of eight types of protein: two recombinant human chemokines with a similar molecular weight, RANTES (CCL5, 7.8 kDa MW) and CXCL1 (8 kDa MW); bovine serum albumin (BSA, 66.5 kDa MW) with a much higher molecular weight; four biotinylated, subtly different variants of the N-terminal tail peptides of histone H3—two of these peptides were native (denoted as H3.2, 15.6 kDa and H3N, 2.5 kDa) and the other two were chemically modified at lysine-9 either by acetylation (H3A) or trimethylation (H3M); and the glycosylated Fc fragment of the antibody IgG4.⁽¹²³⁾ The same analysis was applied to every type of these proteins. When a dilute concentration (300 pM) of denatured protein with SDS and BME was introduced on the cis-side of a pore, blockades were observed in the open pore current that were attributed to the translocation of single molecules. Generally, no blockades were observed in controls that comprised only the electrolyte and the denaturants.

The small cross-section of a sub-nanopore was the key to chemical specificity. To facilitate comparisons and diminish the dependence of the pore current on voltage and electrolytic conductance, the distribution of blockades was classified by the fractional change in the pore current relative to the open pore value (Δl/lₒ) and the duration of the blockade (Δt). Naively, Δl/lₒ can be related to the ratio of the molecular volume to the pore volume: i.e. ΔVₘₒₗ/Vₚₒᵣₑ.⁽⁹⁰⁾ For example, native CCL5 has 68 AA residues, so if the denatured protein has a rod-like shape, about 22 AAs would span the entire membrane and the occluded volume would be about Vₘₒₗ=3.5 nm³. A pore 1.5 nm in diameter with θ=15° in an 8 nm thick membrane has an estimated open volume of Vₚₒᵣₑ=43.8 nm³ so that for a denatured protein, Δl/lₒ=0.0698. This estimate is a lower bound since it neglects features such as the hydration shell and a persistent, native-like topology. However, if the effective thickness of the membrane is defined by the current crowding associated with the bi-conical topography, only four AAs would span a thickness of 1.5 nm, and so the occluded volume would be about ΔVₘₒₗ/V^{eff}ₚₒᵣₑ=0.64 nm³/3.4 nm³=0.19˜Δl/lₒ. Following this reasoning, we expect 0.07<Δl/lₒ<0.19, 0.17<Δl/lₒ<0.94, and 0.22<Δl/lₒ<1 for 1.5-, 0.5- and 0.3-nm pores respectively.

Heat maps were prepared derived from the blockade distributions associated with CCL5 translocations collected from different pores: one with a (1.4±0.1)×(1.6±0.1) nm² cross-section; another with (0.5±0.1)×(0.6±0.1) nm² cross-section; and a third with a (0.3±0.1)×(0.3±0.1) nm² cross-section (FIGS. 2g-i). For a 1V bias, the median fraction Δl/lₒ improves substantially from Δl/lₒ=0.07 to Δl/lₒ=0.38 to Δl/lₒ=0.47 as the pore cross-section was reduced, while the median duration hardly changed, ranging from Δt=400 μs for the 1.4×1.6 nm² pore to Δt=520 μs for the 0.3 nm-diameter pore. Taken together, the Δl/lₒ measured the molecular volume occluding the pore and that the molecular velocity is about the same regardless of the cross-section of the pore. To account for the differences in the duration and fractional blockades, it was assumed that each protein translocation explored different aspects of the pore topography through: 1. different alignments of a rigid, rod-like protein relative to an irregular pore with a sub-nanometer-cross-section; and 2. different trajectories for hydrated ions moving through a blockaded pore of comparable size. Thus, the blockade distribution was attributed to factors relating to conformational noise such as a persistent, native-like topology in the denatured protein unraveling in the pore,⁽¹²²⁾ or the initial configuration of the molecular termini relative to the pore, or different orientations (N-terminus versus C-terminus or yaw/twist about the vertical axis) of the rigid, rod-like molecule relative to the pore topography.

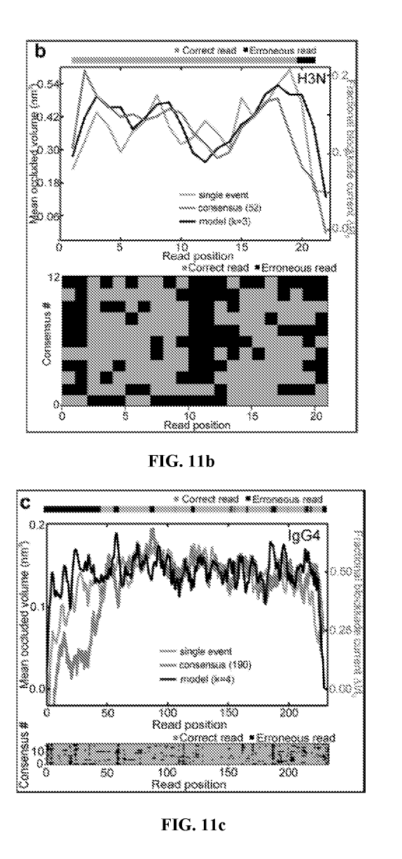

Scrutiny of fluctuations observed within each blockade exposed signatures of the protein sequence. Each blockade within a subset of the distribution from Δl/lₒ>0.30 for 1<Δt<70 ₘₛ comprising the majority of blockades, revealed nearly regular fluctuations beyond the noise, the number of which corresponded closely to the number residues in each type of protein (FIG. 11). The Fourier amplitude associated with the fluctuations varied by more than an order of magnitude, and it was frequently more than four standard deviations beyond the noise found in a trace of the open pore current of comparable duration. In particular, a tally of the number of fluctuations in blockades associated with translocations of denatured CCL5 yielded N_{CCL5}=65.0±3.3, regardless of the duration of the blockade (FIG. 3c), which coincided with the 68 AA residues in the mature protein. Likewise, for a protein with a similar length, CXCL1, we tallied a similar number, N_{CXCL1}=62.6±9.3, corresponding to the 71 residues in the protein. In contrast, a much larger number of fluctuations, N_{BSA}, was 602.0±64, was observed when denatured BSA blockaded the pore, which agreed within the error with 583 AAs in the mature protein. Far fewer fluctuations, N_{H3N}=20.5±1.3, were observed when denatured H3N was impelled through a sub-nanopore, corresponding to a peptide with 21 residues.

Although Fourier analysis was the primary means for counting fluctuations in a blockade, as a check, another method was used to identify and tally them that employed a Gaussian fitting algorithm. It gave similar results. For example, for the traces depicted in FIGS. 11 a,b, the counts (orange circles) were N_{CCL5}=67, and N_{H3N}=21. When the same algorithm was applied generally, it yielded N_{CCL5}=60±29 peaks for CCL5, N_{CWCL1}=62±26 for CSCL1, N_{H3N}=22±12 peaks for H3, and N_{BSA}=601±152 peaks for BSA, which were all consistent with the number of AAs in the proteins within the error. Since fluctuations like these were not observed in pores with cross-sections >0.7×0.8 nm² or in the absence of SDS, the pore topography and the denaturation agents were determined to be causative factors.

Beyond Fourier analysis and Gaussian fits, to account for non-uniformities in translocation velocities, improved, kernel-based methods for detecting fluctuations will be developed to mine the blockade current data. For example, additional information may be gleaned from the jitter associated with the position of the fluctuations in time. Both the flexibility and the size of the AAs comprising the strand likely will affect the translocation velocity causing a jitter in the position of a fluctuation. To account for jitter, first a mean velocity will be estimated for the AA residues to determine the mean delay between fluctuations, τ. Then, starting from the first position, a search will commence for the maximum fractional blockade within the window [t₁+(1−α)t, t₁+(1+α)], where 0<α<1, until a second maximum is located. The location of all subsequent fluctuations will be determined similarly. Larger AAs are expected to have lower mobilities so that the duration Δtᵢ=tᵢ−tᵢ₁ might be correlated with the AA volume. If so, then systematic velocity changes between AAs might be estimated by fitting a regression such as LOESS to Δtᵢ, Δt₂, . . . Δtₙ.⁽¹²⁶⁾

Translocation Kinetics: Because of the correspondence between the number of fluctuations and AA residues in the protein, each fluctuation was determined to reflect a read of the AA sequence of a single protein molecule. Since all the blockades were recorded using the same bandwidth, the observation that both the number of fluctuations and the patterns within a blockade persisted over a range of durations and fractional currents for most of the blockades, precludes random noise as the sole explanation for the fluctuations. Moreover, the analysis of the fluctuation patterns indicated two distinct groups, which showed similar maxima under temporal inversion. This was interpreted as evidence of two nearly equivalent, but opposite, translocation directions—either N-terminus or C-terminus first. Therefore, all the blockades were sorted into two groups and the second was inverted in time. Other discrepancies between the patterns observed in the majority of blockades were attributed to misreads such as a skip or multiple reads of the same AA. Misreads give rise to a different tally of fluctuations, which may be attributed to conformational noise. For example, lags were also observed in the fluctuation pattern. These accounted for 5-25% of the blockades and were ascribed to time-consuming reconfigurations of the molecular termini prior to insertion in the pore. Finally, multi-level events associated with the protein unfolding in the pore were also observed, especially if the SDS was <0.0001%. These accounted for <5% of blockades.

Since the fluctuations were nearly regular, SDS was concluded to impart a nearly uniform negative charge density along the polypeptide resulting in a consistent electric force working to impel the molecule through the pore. It is likely that counter-ions moved along with the protein molecule to minimize Coulomb repulsion, but since a pore has a diameter comparable to a hydrated ion, the motion of the ensemble through it would be impeded by steric hindrance. Thus, the coincidence between the number of fluctuations and AAs in the protein, and the near-regularity of the patterns were consistent with a tightly choreographed, turnstile motion of AAs through the pore in which a single AA stalls repeatedly in a well-defined conformation within the pore and then eventually proceeds through it due to the electric force. This type of motion has actually been observed in experiments that simultaneously measure the force and blockade current characterizing the translocation of a single H3.2 molecule through a sub-nanopore (FIGS. 12a,b).

With a single H3.2 molecule tethered to the tip of an AFM cantilever, the force and current were recorded as the AFM tip was systematically retracted a constant velocity (4.00 nm/s toward the N-terminus) from the membrane until the molecule vacated the pore. Infrequently, both the force and the current measurements reflected the capture and evacuation of the molecule by the pore. A dichotomy was observed in the translocation kinetics: the protein either slid frictionlessly through the pore or it stuck-and-slipped (FIGS. 12 c,d). For example, as the cantilever was retracted from a (0.8±0.1)×(1.3±0.1) nm² pore against a potential of 0.85 V, occasionally force plateaus were observed during the translocation of a single molecule (FIG. 12c). As illustrated in the figure, as the tip was retracted from the pore, adhesion to the silicon nitride membrane predominated for tip-surface separations <48 nm. However, when the tip was released from the surface at position (1), a force plateau was observed at 248±4 pN as a single H3.2 molecule translocated through the pore, as indicated by the blockade current of Δ/=40.9±2.0 pA.

Constant force plateaus like these were interpreted as the molecule sliding relatively “frictionlessly” through the pore. Plateaus in the force were also observed without the protein in the pore as the molecule was peeled from the surface of the membrane without any indication of a blockade in the current. Such plateaus have also been reported when single-stranded DNA (ssDNA) was impelled through a nanopore in a silicon nitride membrane,⁽¹²⁷⁾ but not for protein. Using an AFM, native proteins tethered to an AFM cantilever have been unraveled to inform on protein folding and corresponding function,⁽¹²⁸⁻¹³⁰⁾ but a frictionless slide has not been observed before possibly because prior work has not focused on a denatured protein-SDS aggregate. Lacking any secondary structure, it seems likely that a denatured protein, aggregated with SDS, would produce a featureless force-extension characteristic. On the plateau, the residual forces may be associated with a combination of a relatively weak (W<F·Δl=2.5×10⁻²⁰ J≈3.6 kJ mol⁻¹AA⁻¹) hydrophobic adhesion between the aggregate structure and the membrane, the electrophoretic force and the electroosmotic flow impelling the molecule through the pore.

A second category of translocation kinetics was observed under similar conditions as the cantilever was retracted from another pore with a (0.8±0.1)□(1.0±0.1) nm² cross-section (FIG. 12d). After the tip was released from the surface at position (1), a single H3.2 molecule was pulled with an initial force of 94.7±10.9 pN at a constant velocity of 4.00±0.02 nm/s against a potential of 0.7 V while occluding the pore, giving rise to a blockade Δ/=169±26 pA. The blockade current indicates an occluded volume larger than a denatured protein, which may be attributed to a persistent, native-like topology of the protein either clogging the pore or sticking to the membrane. The protein was subsequently stretched by the differential force ΔF=37 pN over a distance of 11.5 nm until the bond ruptured at position (2), after which it slid frictionlessly until it vacated the pore at position (3) and the current recovered to the open pore value. The associated kinetics through the pore resembled a “slip-stick” motion in which the polymer rapidly slips as soon as the applied force exceeds the threshold for rupturing the adhesive bond between the aggregate and the membrane. Usually, the loading of a single molecule like this produces a force-extension curve that reflects the elasticity of the molecule. Stretching events like these in unstructured protein can be described by statistical mechanical polymer elasticity models, of which the most commonly used are the freely jointed chain (FJC) model.⁽¹³¹⁾ Using the FJC, with a Kuhn length of b ˜0.31-0.56 nm, the effective spring constant associated with each stretching event was estimated from k_{eff}=3 k_{B}T/bX, where k_{B}T represents the thermal energy at 293° K and X is the extension of the protein relative to its total length to find k_{eff}=3.2±1.6 pN/nm, which is consistent with prior estimates.⁽¹³²⁾

These results on molecular transport through the pore are consistent with MD simulations of the translocations of unfolded proteins. MD reveals that an unfolded protein collapses to the surface of the membrane (from an initially extended conformation over the pore) almost immediately (within 30 ns) as it equilibrates.⁽¹³³⁾ Nevertheless, despite being absorbed, the peptide chain continues to diffuse along the surface. However, electric charge on the membrane affects the transport and can force a charged polymer to adopt a range of different conformations on the surface. Similar effects have also been observed in MD when other charged biopolymers like DNA interact with smooth hydrophobic surfaces such as graphene when the electric charge density is altered.⁽¹¹¹⁾ In particular, on a charge-neutral membrane, the nucleobases of ssDNA adhered to the surface, but the negatively charged phosphate backbone does not. For a negatively charged surface, ssDNA was observed to unbind from the surface, indicating the predominance of electrostatic repulsion between the electronegative phosphate groups of DNA and the negatively charged surface, promoting frictionless slides. Since the negatively charged protein-SDS aggregate has similar properties as ssDNA, such as regularly spaced negative charges and aromatic rings, the transport properties should be similar too. Thus, the balance of hydrophobic and electrostatic forces on the membrane will be susceptible to fine-tuning affecting transport through the sub-nanopore.

Controlling Translocation Kinetics with the Membrane. To balance the forces, the hydrophobicity will be controlled using a combination of different membrane materials and surface treatments, and manipulate the electrostatics over the pore using an embedded electrode. Graphene is a 2D material with a thickness of 0.34 nm that is supposed to be strongly hydrophobic, since the carbon lattice appears perfectly nonpolar.⁽¹³⁴⁻¹³⁶⁾ However, recent work has indicated otherwise perhaps illustrating the role defects and contaminates may play.⁽¹³⁷⁾ Silicon nitride is also hydrophobic, but less so, whereas silicon dioxide is hydrophillic. Silicon dioxide acquires a negative surface charge density through the dissociation of terminal silanol group that makes it hydrophillic. Hydrophillic surfaces typically lead to poor adhesion, but the surfaces of these materials are often chemically modified to affect the hydrophobicity, electrical charge and solvation. For example, a combination of hexamethyldisilazane (HMDS) and trimethylchlorosilane (TMCS) in a range of concentration from 3-12% demonstrated a control the hydrophobicity of silicon nitride membranes. In addition, organosilane agents such as (3-aminopropyl) triethoxysilane (APTES), may be used to prepare positively-charged amino-terminated films. Following standard protocols, silanization can be used to enhance the hydrophobicity of the surface to an extent depending on the monolayer coverage. To relieve non-specific binding of protein, pegylation (PEGylate) of the surface of the microfluidic channels, and covering the glass and silicon chip containing the pore using mPEG-silane (mPEG-Si), may be implemented. The PEG forms a stable interface layer that inhibits interactions between the surface and proteins, while maintaining relatively high surface hydrophilicity. All of these surface treatments may be provided in various embodiments of the invention to fine-tune hydrophobicity.

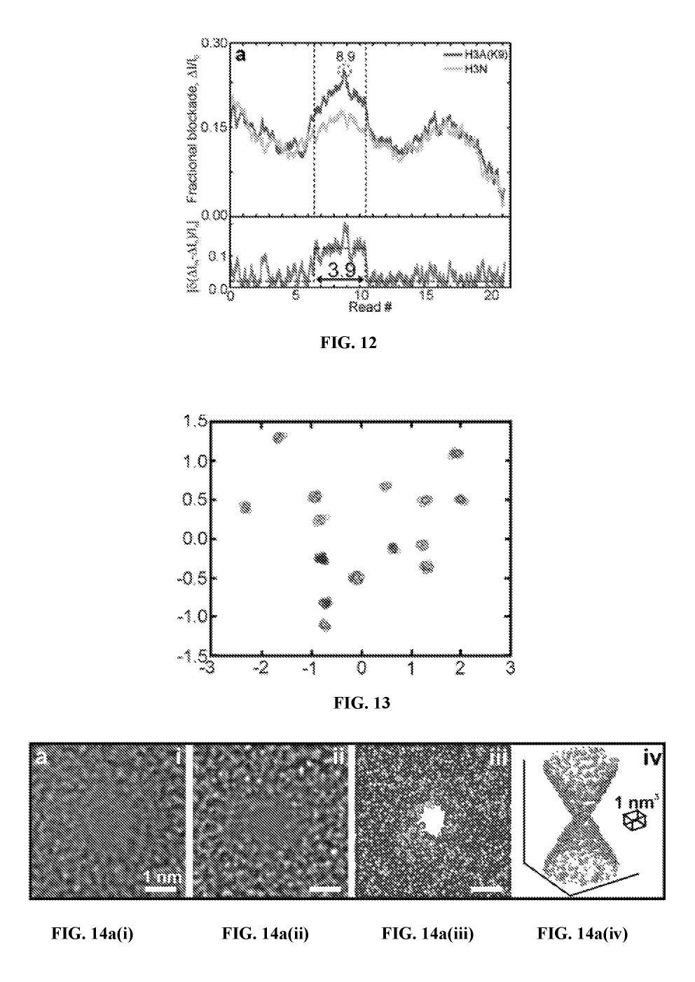

Because of the limited extent of the electric field over a sub-nanopore, to more effectively control the electrostatics silicon microfabrication techniques will be used to produce Ag/AgCl annular electrodes encircling the membrane/pore (FIG. 13). The electrodes allow for control of the electric field and, at the same time, improve high-speed and noise performance by reducing the series resistance. Since the resistance scales like (¼r)(1/t), where r is the radius and t the exposed thickness, an electrode 1 μm thick, encircling a 2×2 μm² membrane, should lower the resistance 200-fold.

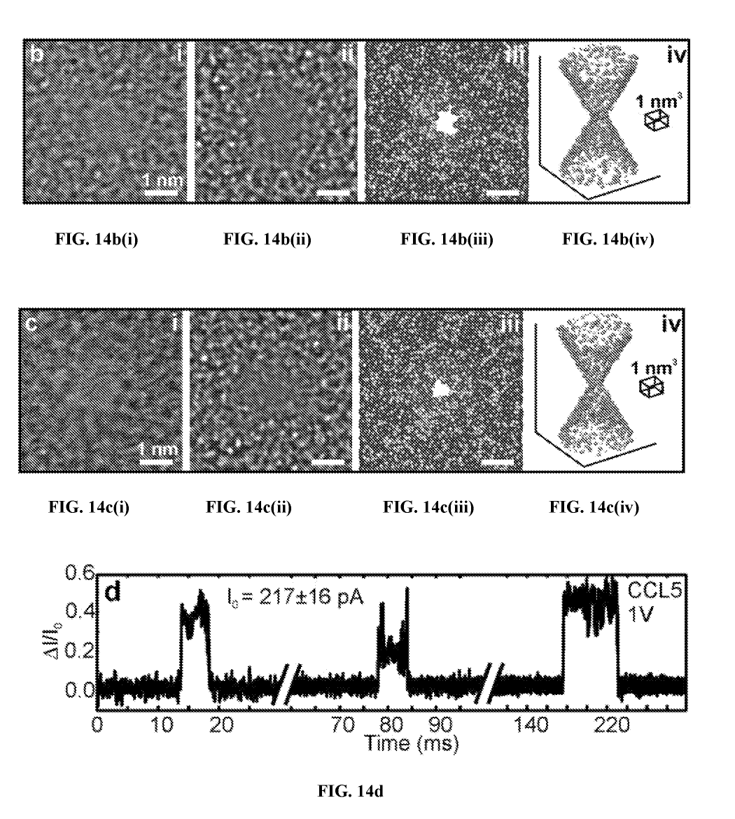

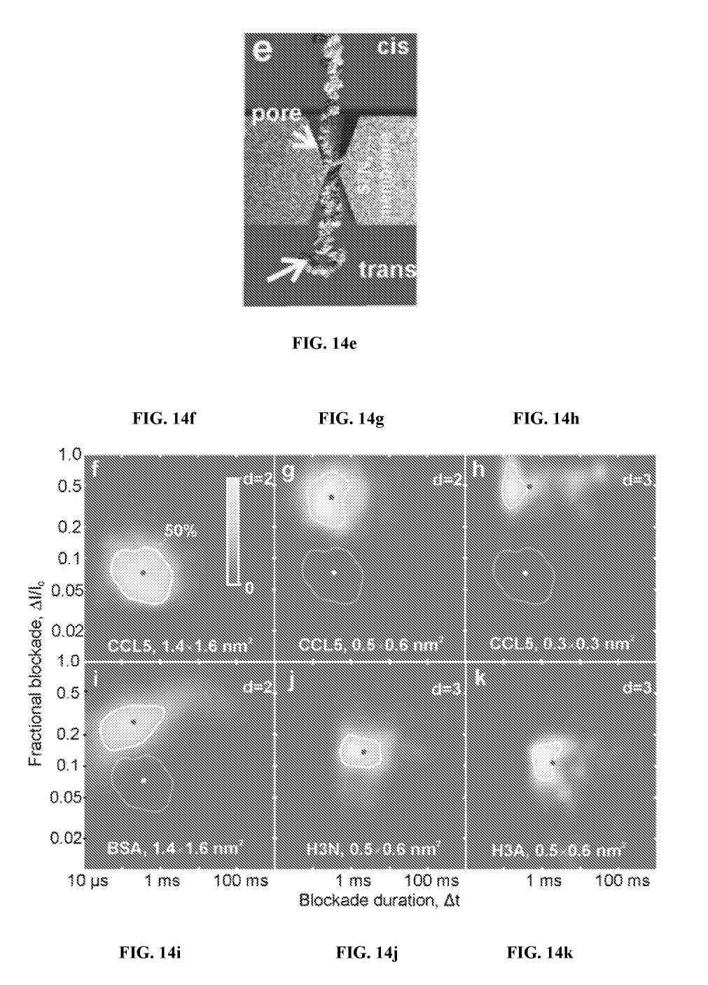

Detecting Quadromers: In the subset of data for which the translocations were frictionless, scrutiny of the force and current fluctuations revealed regular correlated patterns intermittently (FIG. 14), but exclusively in sub-nanopores with diameters <0.8 nm. Focusing exclusively on the subset associated with relatively frictionless kinetics through a sub-nanopore, FIG. 14 represents data acquired when a molecule was retracted at a constant velocity of 4.0 nm/s from a (0.6±0.1)×(0.7±0.1) nm² against 0.7 V. After the tip was released from the surface, the molecule stuck to the membrane at position (1), at a tip height of about 3.8 nm, corresponding to a blockade current of Δ/=33.5±6.4 pA, indicating that a single molecule blockdaded pore. Starting at a force of 120.7±9.8 pN, as it translocated through the pore, the protein slipped-and-stuck twice, stretching between positions (1) and (2) and then again between (2) and (3). Subsequently, the force was relatively constant until position (4) where the molecule slipped-stuck three more times. Finally, at position (7), the force on the molecule was relieved abruptly while the current remained blockaded before returning to the open pore value at position (8). Since the current returned to the open pore value after the force was relieved, it was inferred that the SA-biotin bond ruptured prematurely and the molecule remained for an additional ˜1.5 s before vacating the pore.

The molecule slid relatively frictionlessly through the pore in the gap between 15.5 and 23.5 nm. In that interval, periodicities were discernible in magnified views of the force and current (FIG. 14b), but they were even more apparent in the autocorrelation functions (ACFs) of the data (FIGS. 14 c,d). The force exhibited intermittent regular oscillations (FIG. 14c; left) at a mean lag of 0.58±0.06 nm, while the current showed a mean lag of 0.47±0.07 (FIG. 14d; left), which corresponds closely with the distance between stretched residues. With 135±5 pN of tension applied, corresponding to the modulus of the denatured protein, the distance between residues was stretched from the equilibrium distance of 0.38 nm to about 0.5 nm. The separation between peaks in the force and current ACFs were nearly equidistant over the 10 nm range, as evident in the kymographs (FIGS. 14c,d; right) that represent a compilation of ACFs using a 3.0 nm moving window with a starting position that is staggered by 0.02 nm. The regularity suggests a correlation between the orientation and topology of the residues in the pore. Taken altogether, these data support the idea that the frictionless translocation of protein through a sub-nanopore occurs in steps that are subject to control by the applied force. On the other hand, pores with larger cross-sections exhibiting force plateaus did not show evidence of correlations in the force or blockade current beyond the noise. Moreover, even after the baseline stretching force was subtracted, the residual force and the blockade associated with a single protein stretching in a pore did not exhibit regular fluctuations like those observed on frictionless plateaus.

If each fluctuation in a blockade reflects an event in which one AA enters the pore while another leaves, then it was reasoned that the amplitude of the fluctuation should be attributed to the occluded volume associated with the AA residues in the pore. Because the pore current was crowded and most of the potential dropped near the waist, each fluctuation should measure the occluded volume due to 3-5 AAs (depending on the pore topography), with the exception of the first and last fluctuations at the inception and termination of a blockade. These were interpreted as a reduced sum of AAs. This was examined by correlating the blockade current, measured when the molecule was impelled systematically by AFM through a sub-nanopore, to a model for the protein in which each AA was represented by its volume estimated from crystallography data.⁽¹⁴¹⁾ To account for the current crowding as the molecule steps successively one AA at a time through the pore, a moving average with a window size spanning from k=3 to 5 AAs long (depending on the pore) was performed on the sequence of volumes (FIG. 14e).

The model based on AA volumes was found to be strongly correlated to the empirical data on H3.2. In particular, for the data acquired from a 0.6×0.7 nm₂ pore, the Pearson correlation coefficient C=0.52 for k=4, where k denotes the number of AAs in the pore waist. The band above the plot in FIG. 14e represents the agreement between the empirical data and the model for each read expressed as gray (black) for correct (incorrect) calls, depending on whether the agreement was greater or less than 20%, respectively. The read accuracy from a single molecule was about 75%, more than 56 (standard deviations) above the reads acquired by fitting noise. There are several qualifications required on the read accuracy. First, the number of correct reads obtained for each blockade does not reflect the accuracy with which single residues can be called. The threshold for a correct read was chosen to be 20%, which means that, on average, ±20% of optimally ranged and fitted random noise would fit the model because 40% of all data will fall within its threshold boundary. Nevertheless, the read accuracy is statistically significant as per the null sets determined in prior work.⁽¹⁰¹⁾ Thus, each fluctuation represents a (low fidelity) read of the volume of a quadromer in the waist of the pore.

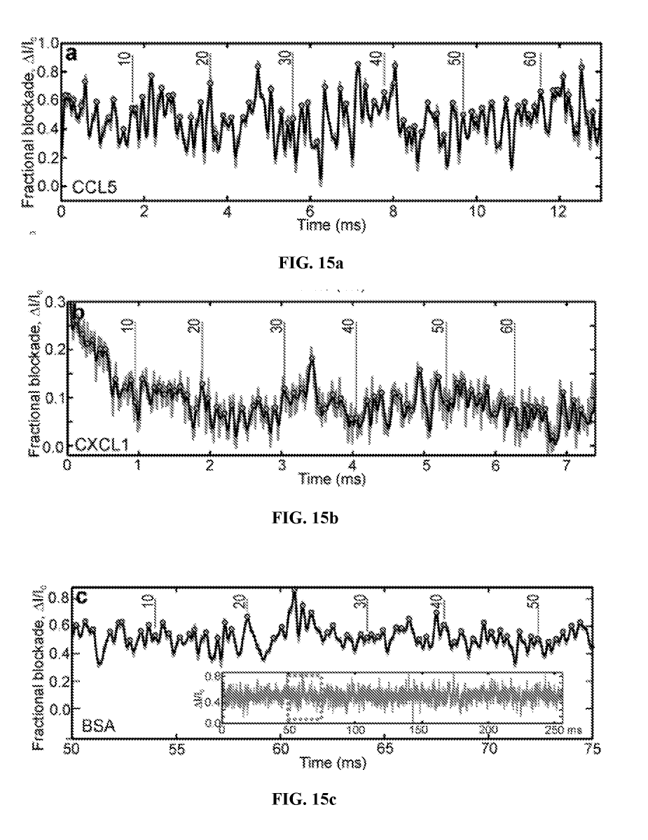

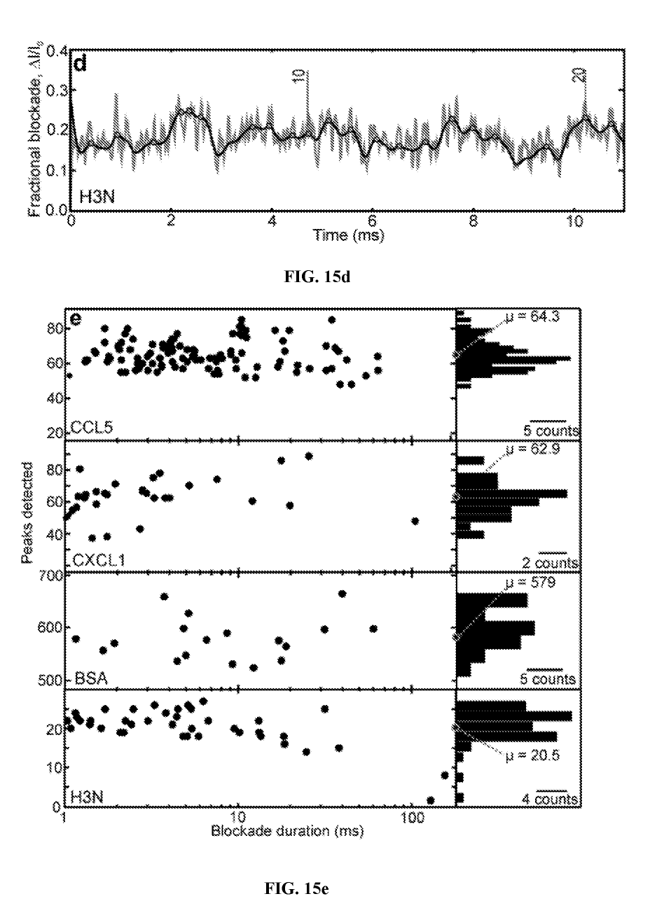

The correlation between the model and the empirical data persists even without the systematic control of the translocation kinetics provided by the AFM (FIG. 15). However, for comparison, since neither the duration nor the fractional current was perfectly uniform, each blockade was normalized in time and the average fractional current was zeroed (FIGS. 15a-c, blue lines). A consensus was then formed from the average of a number of blockades (red lines)—each associated with the translocation of a single molecule. The mean correlation coefficient between the consensus and an individual blockade was 0.42 for CCL5, 0.55 for CXCL1 (FIG. 15a), 0.67 for H3N (FIG. 15b), 0.48 for IgG4, and 0.23 for BSA. Thus, the fluctuations persisted even after averaging, unlike the noise.

The model based on AA volumes was well correlated to the empirical consensuses, and the agreement improved as the number of blockades included in the consensuses increased. For example, error maps were produced by partitioning 45 CXCL1 blockades into nine consensus (FIG. 15a, bottom), each of which was compared to a k=5 model. While the correlation to an individual blockade or short consensus was lower, the correlation of the model with the entire 45-blockade consensus was 0.68, with an associated mean percentage read accuracy of 84.7% for a 20% threshold tolerance. Thus, increasing the coverage by including more blockades into the consensus improved the read accuracy. The performance on the shortest peptide, H3N, was similar. The correlation of the k=3 model with a single event was 68%, but a 52-blockade consensus showed only two positions in error out of 21—a read accuracy of 90%. Likewise for IgG4, the correlation of the k=4 model with a single event was 48%, but a 190-blockade consensus showed C=67% (84% beyond the divergence near the N-terminus.)

Regardless of the coverage, read fidelity could still be compromised by systemic errors, however. A further analysis of the read fidelity and cross-correlation between proteins exposed several interesting trends. For example, it was apparent from the error maps (FIGS. 15a-c, bottom) that the correlations to the model do not accumulate randomly; e.g. discrepancies were consistently found near positions 6, 12, 29 and 30 for CXCL1 and positions 11, 12 for H3N (although these were suppressed in the consensus) and at positions 59 and 187 for IgG4. By assigning errors to the a-priori sequence of cumulative AA volumes at each position, the volume error for each AA was calculated that, taken together, indicated the source of the errors. In particular, negatively charged AAs (D, E) repeatedly showed the highest read errors for these three proteins. On the other hand, the two (positively charged) lysines (K) at position 54-55 both exhibited a mean read accuracy of 92% for the 17 separate CCL5 runs. Finally, AAs with small volumes (A, C, G, and S) were frequently misread, which can be rationalized because the AA volume was only <10% of the effective pore volume.

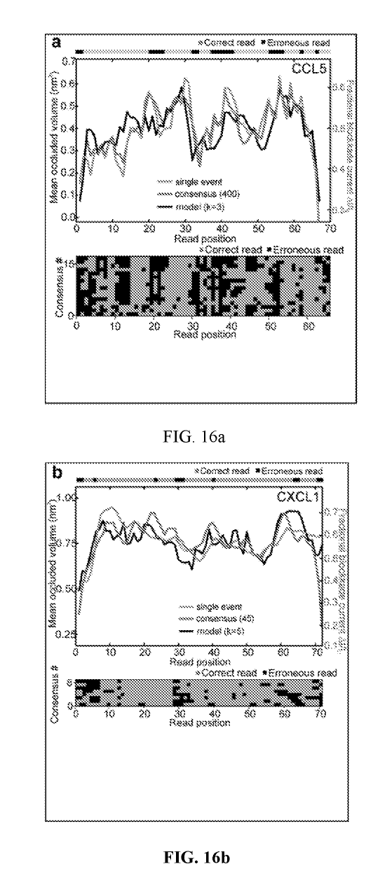

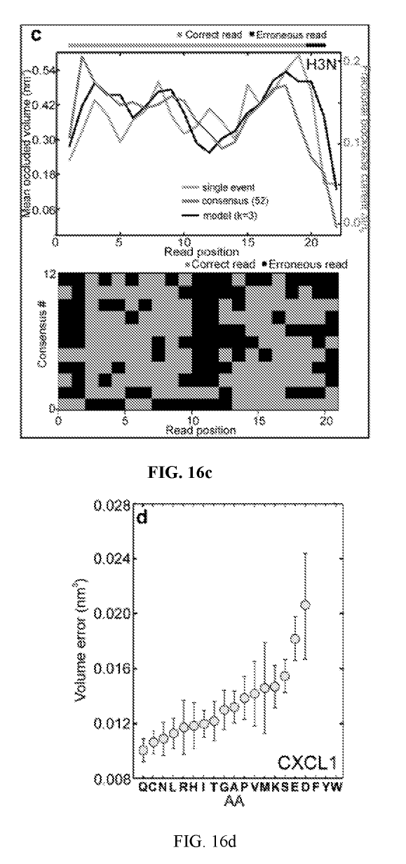

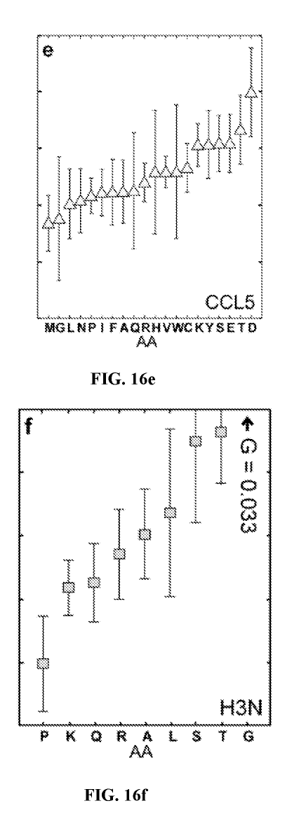

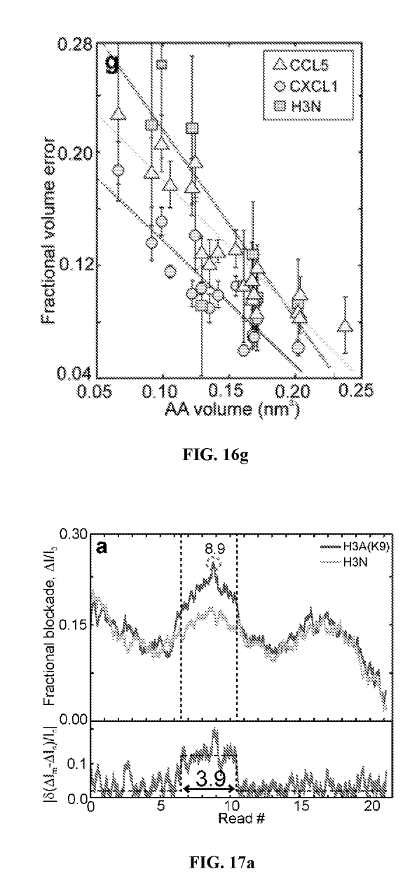

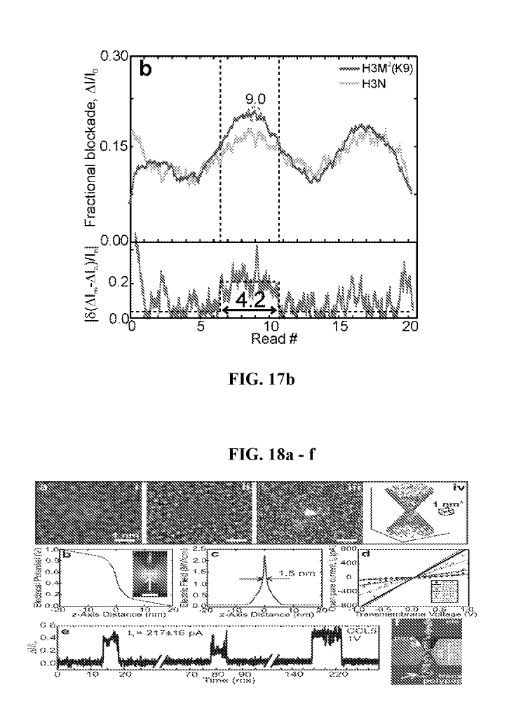

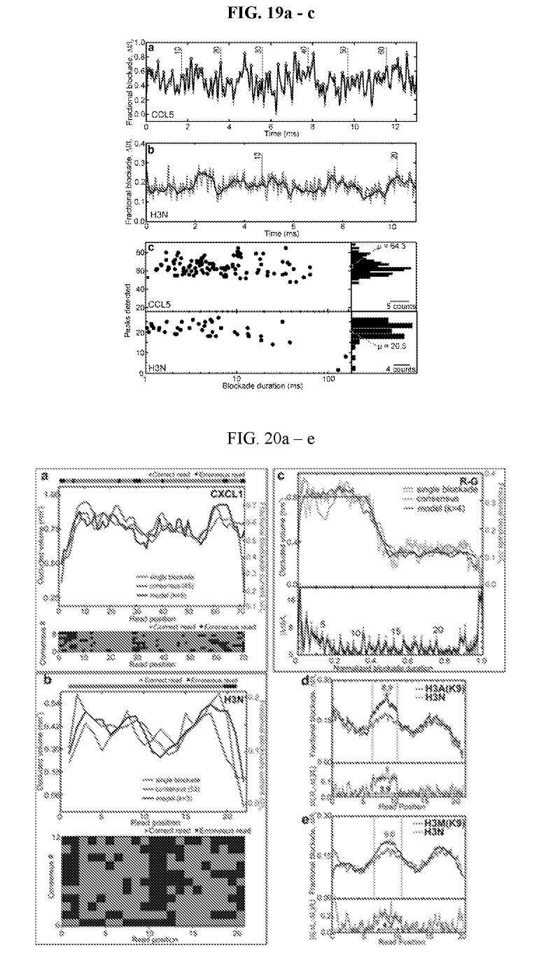

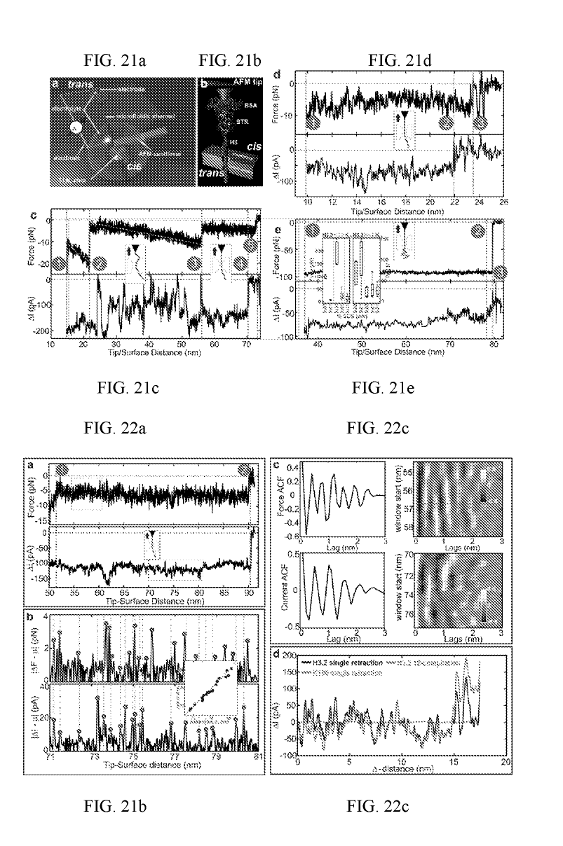

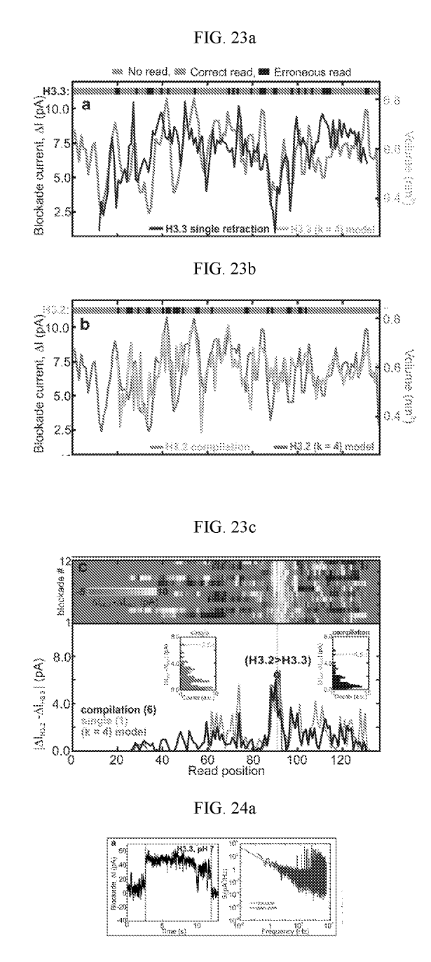

The relative insensitivity to AAs with small volume cannot be remedied by testing with short homopolymers because homopolymeric AA tracts are often involved in protein-protein interactions and have polymerization properties that might confound the interpretation of the blockade current.⁽¹⁴²⁾ Instead, the sensitivity was tested using PTMs of specific residues. PTMs such as acetylation, methylation, and phosphorylation introduce new functional groups into the peptide chain that extend protein chemistry beyond the twenty proteinogenic AAs. To measure the sensitivity to PTMs, three variants of the tail histone H3: H3N, H3A, and H3M, were analyzed and compared (FIG. 16). These modifications were especially interesting in the context of a blockade current measurement because the changes in the occluded volume were expected to be like that associated with G. For comparison, three consensuses were formed: one associated with 304 blockades of H3; and another with 231 blockades from H3A (FIG. 16, top) and a third with 958 blockades from H3M, each was acquired from a 0.5-nm-diameter pore at 0.7 V. Subsequently, consensuses for the conjugates were ranged to the mean blockade of the H3N.

The juxtaposition of consensuses clearly showed the positional sensitivity of the fractional blockade current. The fractional blockade associated with both chemical modifications was enhanced between read positions 6 and 11. In addition, a prominent feature was observed near read position 9 in H3A measured relative to H3N, in correspondence with the expected change on K9 due to acetylation. In contrast, a depression appeared near read position K9 in H3M measured relative to H3N. Fitting the difference in the fractional blockade between the chemically modified and native traces to a top-hat form revealed differences beyond the noise over a range of 3.9 positions (FIG. 16, bottom), which substantiates the claim that each read reflects about four AAs. Likewise, the difference in the fractional blockade between the H3M and H3N traces extended over a range of 4.2 positions. Therefore, based on the sensitivity to PTMs of single AAs, the fluctuations in the blockade current measure the moving average of the occluded volumes of a quadromer.

Resolution through Improved Pore Topography. Due to the extraordinary chemical specificity of a sub-nanopore, the position of single PTMs can be accurately read within a quadromer from a consensus of blockades. However, reading a monomer directly would facilitate the interpretation of the data. To improve on the chemical specificity so as to reduce the number of molecules required in a consensus, and read with higher resolution, the following actions/modifications will be provided to the membranes. First, the membrane will be made thinner to narrow the range of current crowding. Si₃N₄ membranes 5 nm thick and SiO₂ membranes 2 nm thick have already been created herein. Membrane topography with sub-nanopores having a larger cone-angle will produce a more tightly focused electric field focus. Membrane of a molecular sheet fashioned from graphene, 0.34 nm thick, or a single layer of MoS₂, that is 1 nm thick, constitute alternative materials for the membranes according to other aspects of the invention. MD indicates that the graphene thickness will only support a few conductance states—it is probably too thin to distinguish all the AAs, although MoS₂ may show more states. Moreover, AA may stick to hydrophobic graphene, but not necessarily to hydrophilic MoS₂.⁽¹⁴³⁾ Thus, all four: Si₃N₄, SiO₂, graphene and MoS₂ will be tested as membranes.

Large-scale DP to decode the sequence. Just like the biological nanopore sequencers used for DNA, low fidelity reads with multiple monomers affecting the blockade current should not pose a problem for sequencing protein with single AA resolution, provided that the translocation rate is controlled and the coverage is high. Three candidate methods may be used in the present methods for calling individual AAs from the blockade current: 1. a Hidden Markov Model (HMM);⁽¹⁴⁵⁾ 2. an ensemble tree-based method such as MART (Multiple Additive Regression Trees)⁽¹⁴⁶⁾ or random forest; and 3. an L1-penalized (Lasso) logistic regression.⁽¹⁴⁷⁾ On the one hand, the HMM approach is well suited to the turnstile-like format of the data and offers the advantage of decoding the whole sequence, but the prima fact complexity of the algorithm seems unwieldy for protein, as the state space grows exponentially with increasing numbers of AAs and PTMs. On the other hand, regression-based methods can incorporate many features, including information from nearby fluctuations, to achieve similar power to HMMs with less computational burden.

Furthermore, L1-penalized regression does variable selection automatically and offers a model that is easy to interpret; it is preferred over tree-based methods. Regression-based methods not only give the most-likely call for each fluctuation, but also the likelihood of each AA, which could be useful for follow-on analysis. Finally, efficient statistical algorithms will be deployed to identify insertions and deletions (INDELs). Using the likelihood for each peak being one of the possible AAs, the overall likelihood for each alignment will be established and then a search for the best alignment will ensue. The optimal sequence alignment is already a well-studied problem⁽¹⁴⁹⁾ and a number of efficient algorithms have been developed that produce a p-value indicating the confidence about the identification of an INDEL.⁽¹⁵⁰⁾

HMMs will be used in some embodiments of the methods to represent AAs successively moving through the pore, affecting current blockade measurements and so it could be adapted to call AAs from the signal. As the AAs move through the pore, there are changes in state, which can be captured by a chain of hidden states only observed indirectly (via the current). For a HMM, a state diagram can be constructed from output probabilities, the probability distribution of currents observed for each state, and transition probabilities (5% if each AA has an equal probability.) It is then possible to maximize the joint probability, using the entire chain of observed currents to determine the hidden state chain. The joint probability is given by P(1(t)|k)×Tⱼₖ where P(1(t)|k) is the output probability for state k, and Tⱼₖ is the transition probability between states j and k. The total joint probability is given by δₖΠₜP(1(t)|kₜ)×T₍ₜ₋₁₎₍ₜ₎ where δₖ is the probability that each of the states is initially occupied. A Viterbi algorithm can used to determine, at each step, the most probable combination of previous steps to reach that point, given by: Vₖ(t)=P(1(t|k) maxⱼ(Vⱼ(t−1)×Tⱼₖ). Using this method the sequence of (in this case, quadromer) states can be inferred to delineate the protein sequence. In principal, this method can be used on any protein, by expanding or contracting the number of possible states, depending on the number of monomers and PTMs influencing the blockade current. However, this model does depend on having the AAs advancing one at a time through the pore. If not, the transition probability matrix also has to be expanded to include the probability of a repeat of the same state, or advancing two monomers (a skip).⁽¹⁴⁸⁾

HMMs with Supervised Learning. The number of residues affecting the blockade current, compounded with the SNR, presents a challenge for calling individual AAs in the sequence. Unlike DNA, the multiplicity of states associated with twenty proteinogenic AAs is confounding. Consider the situation where four AAs, a quadromer, affect the pore current—this translates to 204 or 160,000 different possible combinations or states affecting the blockade current level, which will not be easily discriminated due to the SNR. Several strategies will be explored to reduce the computational burden associated with this multiplicity. Initially, a coarse-grain approach will be implemented that uses a reduced set of volumes to call a quadromer. For example, instead of 20 AAs each with a characteristic volume, the fluctuations in the blockade current will be classified into fewer (four) categories to discriminate between AAs with large (>0.2 nm³), intermediate (between 0.15 and 0.2 nm³), small (between 0.1 and 0.15 nm³) and miniscule (<0.1 nm³) volumes. Accordingly, the number of possible states is reduced from 160,000 to a more manageable number, 44=256. With this classification schema, the specificity within the AA sequence will suffer but, nevertheless, it should be to discriminate one protein from another. Then, as the read fidelity or the resolution improves, the complexity of the algorithm will increase to accommodate the new information.

HMMs also require emission probability distributions: i.e. the likelihood of a current measurement corresponding to a (hidden) state. To discover the correspondence, several strategies could be applied. First, the volume model described above could be used to estimate the signal generated by several AAs. Alternatively, since many reads of known proteins are already available, it is possible to allow an algorithm to learn the correspondence by training it on the empirical data. To illustrate how this works, an SVM-based supervised learning approach was used to find a regression of pore signal measurements on chosen AAs features. The algorithm took as input a set of pairs—quadromer and corresponding signal value. It then converted each quadromer into a set of features, for example, a tuple of length four, e.g. (1, 2, 0, 1) corresponded to a quadromer with one miniscule, two small and one large AA. Then an SVM regression analysis was performed to find the correspondence between the features and signal. (This approach offers the benefit that it can be easily extended with other AA properties, such as charge.) Principal component analysis (PCA) of the training set showed distinct clusters of quadromers associated with different signal values (FIG. 9), confirming the robustness of SVM-based approach. Overall, a preliminary implementation of the HMM model showed a 50% correlation with the H3N data (FIG. 7b).

Mining Protein Data Bases. The invention also provides a method whereby the data generated with the methods may be mined to characterize and identify full sequences of a protein from observed frequencies of small amino acid sequence correlation coefficients. Additional information gleaned from the physical and chemical properties of the AAs may impose additional constraints on the transition matrix. For example, information about mobility through a sub-nanopore extracted from analysis of the jitter could be incorporated into the statistical analysis. Moreover, the properties of the protein give rise to correlations between pairs of AA that can be used to discriminate between the possibilities, contracting the transition probability matrix.⁽¹⁵¹⁻¹⁵⁴⁾ For example, if 10-15 residues within a known domain in a protein (represented in Pfam) are sequenced with high fidelity, the entire protein might be recognized, based on the MINI library and the probabilities for the next residues assigned accordingly. In general, for the 20 AAs, there are 190 correlation coefficients. If the occurrence frequency were random, the correlation coefficients between any AAs would be small ( 1/20), but interestingly, some Cs are much higher than average, which could be used to infer about pairs of AAs.⁽¹⁵¹⁾

sub-nanopore tool validation with 3rd generation (nanopore) DNA sequencing. Motivated by the prospects for antibody discovery, several studies have harnessed DNA sequencing for analyzing immunoglobulin repertoires recently. Antibody variable regions and CDRs have been identified, an understanding of how the immunoglobulin repertoire evolved was inferred, and additional mechanisms for expanding the diversity or fine-tuning it were all obtained by sequencing nucleic acids.^{(81, 82, 84)} However, the current platforms suffer limitations.⁽⁸¹⁾ The size of the human antibody repertoire (1011) is beyond the scope of current platforms. Analyzing natural CDR sequences requires long reads, which excludes 2^{nd}gen. sequencing, e.g. Illumina (2×300 bp reads). In contrast, 3^{rd} generation sequencing, including Pacific Bio and Oxford Nanopore platforms, generate long (>4 kb) reads of single molecules that can sequence entire transcripts.^{(155, 156)}

From banked mouse hybridoma cell lines (ATCC), different lines raised against the same antigen, i.e. HB-8507 and HB-8508 against EGF receptor, will be raised, as well as lines against different classes of antigens, i.e. EGF and Coxsackie virus B4 (HB-181). By culturing these cells, the antibodies they produce and their mRNA and DNA will be captured. The antibodies will be purified using a Protein G sepharose column for affinity binding; mouse IgG binds well to protein G. The purified antibodies will be sequenced with a sub-nanopore, and will also be validated with nucleic acid sequencing obtained from the same hybridoma lines. To obtain the sequencing information, conventional 2^{nd} generation sequencing (Illumina, MiSeq) with amplicons targeted to the CDR regions,⁷⁷ combined with unique molecular identifiers, will be used.⁽¹⁵⁸⁾ Unique molecular identifiers will allow for bioinformatic correction of defects due to sequencing or PCR in library preparation.

However, these short reads fall short of full antibody sequencing needed for exploring polyclonal diversity. A 3^{rd} generation sequencing approach coupled with a solution-phase capture methodology will be developed. In this method, the antibody encoding mRNA will be captured using biotinylated anti-sense probes (IDT) against the constant regions of the antibody coding sequence. Using magnetic streptavidin coated beads, all but the antibody producing transcript will be removed, allowing for a high depth of sequencing of this transcript. This short-circuits the relatively low yield that limits current 3rd generation sequencing platforms. Following this step, 2^{nd} strand synthesis will be performed and a 3^{rd} generation sequencing library for the Oxford Nanopore platform will be provided. Whole transcript sequencing will not limit us to only a part of the CDR, instead allowing the identification of the different full antibody primary sequences present. A robust capture methodology for specific transcript sequencing on 3^{rd} generation sequencing platforms does not currently exist, but should not prove intractable to generate. More sequencing of the whole transcriptome may be performed to achieve sufficient sequencing depth of our antibody sequence, enabled by recently developed 3^{rd} generation platforms (PacBio Sequel, ONT PromethION).

With either the CDR coding sequence or the full antibody transcript now available, in vitro transcription will be performed of the sequence to generate the putative protein amino acid sequence. This will be used as a reference to both validate the protein sequencing results from the sub-nanopore, and to determine what translational and post-translational information is encoded in the antibody above and beyond the direct nucleic acid sequence. The sub-nanopore has the exquisite sensitivity required to detect even small unreactive post-translational modifications, e.g. methyl groups. This feature may also be used to further characterize biologically important molecules.

### Example 4—Correlated Transport to Improve the Read Fidelity

The present example demonstrates a correlated transport that improves read fidelity of an amino acid sequence. Also demonstrated in the present example is the significance of both the size of the pore and the size of the ions carrying the blockade current in affecting the sensitivity of the methods for sequencing amino acids. Therefore, if the pore is small enough, it is proposed that the electrolytic ions or hydrate protons cannot carry the blockade current when a protein translocates through it (according to the Grotthuss mechanism).

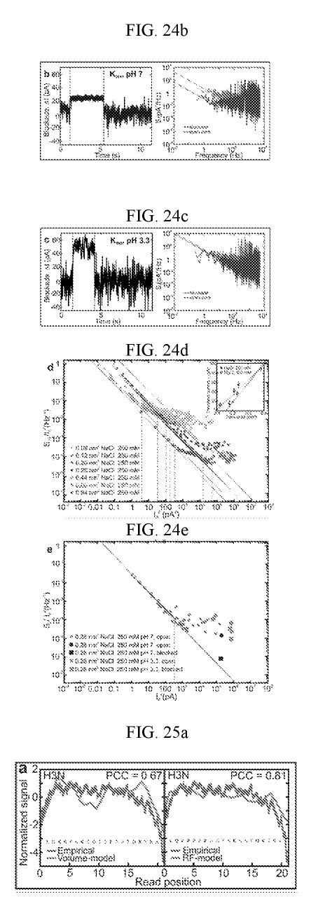

To punctuate the argument that the fluctuations in the blockade measure the occluded molecular volume in a sub-nanopore, current blockades acquired under similar conditions due to the H3.3 variant and K₁₀₀ were also juxtaposed, revealing pronounced fluctuations from H3.3 (FIG. 24b; left), corresponding to a difference the coefficient of variation during the blockade cV_{K}_{₁₀₀}=0.046<CV_{H3.3}=0.271, which again corroborates the assertion that the fluctuations inform on the difference between quadromer volumes. Thus, it seems likely that the chemical specificity of the sub-nanopore is derived from its size in combination with the size of ions carrying the blockade current. However, the cross-section of a sub-nanopore is smaller than the 0.358 nm radius of a hydrated Na⁺ ion used to acquire this data, but comparable to the unhydrated radius (0.117 nm), so the ion is unlikely to be screened by water in the pore. Thus, it was reckoned that ion correlations would play a role in the blockade current and noise. In a statistical analysis, the conductance represents only the first moment of the characteristic quantized charge transport function of the probability distribution, whereas non-equilibrium current fluctuations represent the second moment. So, it was reasoned that the noise would be a more sensitive measure of ion correlations. In this context, noise measurements were performed on differently sized pores to check for correlations.

The current noise was inescapable (FIGS. 24a-c; right) and correlations in it were conspicuous (FIGS. 24d,e). With a voltage bias, the low frequency current noise power spectral density (PSD) had at least two components: a pink (l/f) component and an excess, frequency-independent (white) noise component between 100 Hz and 10 kHz (FIGS. 24a-c). The spectra were classified by fitting to:

$${S}_{I} = {S}_{1 \text{/} f} \frac{1}{f} + {S}_{0} + {S}_{1} f + … ,$$

to extract the parameters, S_{l/f}, S₀, and S₁, that measured respectively the amplitude of the l/f white and dielectric noise. While the noise between 0.1<f<100 Hz was observed to be inversely proportional to the frequency, i.e. S_{l/f}˜f^{-β}, it was not universally so that β=−1, but rather, 0.9±1.1<β<3.0±1.1. On the other hand, when the exponent was forced to fit β=S_{l/f} was found to be independent of the current for Iₒ≤1 pA, which is evident since the normalized current noise Sₗ/I²˜1/Iₒ² (FIGS. 24d,e). These observations were consistent predictions of l/f noise due to diffusion-controlled carrier fluctuations in a point contact.

Because the size of the sub-nanopore was smaller than a hydrated ion, the noise power measured at low current was attributed exclusively to the uncorrelated transport of dehydrated ions—singly, one at a time—through the pore. The PSD is related to the current autocorrelation function: c= [symbol] ΔI(t)ΔI(t+δt) [symbol] , where □I=I(t)□<I> is the noise current and <I> is the average current, through the Wiener-Khintchine theorem. To illuminate the correlations, the noise power was normalized so that: Sₗ/I²= [symbol] ΔI² [symbol] / [symbol] I [symbol] ²Γ(f/f*), where f* denotes a relaxation time. If the average current is given by: [symbol] I [symbol] =Ni, where N is the number of carriers and i is the current carried by a single carrier, then the mean square fluctuations in the current scale like: [symbol] ΔI² [symbol] =N [symbol] Δi² [symbol] , since the individual spectral density components add in quadrature if the ions were uncorrelated, i.e.

$$Var ( {\sum }_{n = 1}^{N} {i}_{n} ) = {\sum }_{n = 1}^{N} Var ( {i}_{n} ) + 2 {\sum }_{n < m}^{N} Cov ( {i}_{n} , {i}_{m} ) .$$

If the single carrier currents are uncorrelated, then the covariance vanishes. Therefore, if N particles carry the current, then [symbol] ΔI² [symbol] =N [symbol] Δi² [symbol] or [symbol] ΔI² [symbol] /I²=(1/N) [symbol] Δi² [symbol] /i², from which it follows that: Sₗ/I₀²˜1/N. Since the data shows Sₗ/I₀² for I₀≤1 pA, it must be that the current was carried by an uncorrelated single file of ions translocating through a point contact, i.e. N=1 so that [symbol] ΔI₂ [symbol] = [symbol] Δi² [symbol] and Sₗ/I₀²˜1/I₀².

As the current increased beyond a threshold, Iₜ, the normalized noise power remained independent of the current across the band, i.e. S_{l/f}/I₀²˜const (FIGS. 24d,e), which implied that [symbol] ΔI² [symbol] =N² [symbol] Δi² [symbol] , signifying the development of correlations in the time-dependent current fluctuations. That the single particle currents were correlated follows from the noise power since the variance of their sum must be the sum of their covariances: i.e.

$$Var ( {\sum }_{n = 1}^{N} {i}_{n} ) = {\sum }_{n = 1}^{N} {\sum }_{m = 1}^{N} Cov ( {i}_{n} , {i}_{m} ) = 〈 Δ {i}^{2} 〉 [ ( N ) + N ( N - 1 ) ρ ] ,$$

where □ represents the average correlation between the single particle spectral densities. Thus, for perfect correlation (p=1), Sₗ/I²˜ [symbol] ΔI² [symbol] / [symbol] I [symbol] ²=N² [symbol] Δi² [symbol] /N²i²˜const. The increase in the PSD above threshold implies that the ions transit the pore in bunches. Shrinking the pore volume, or inflating the ionic radius is proposed to improve the correlations between the single particle spectral densities, and in-line with this assertion, Iₜ, collapsed with the pore cross-section (FIG. 18d; inset.) Thus, it was inferred that a K₁₀₀, blockade would reduce the unoccluded volume, depress the current threshold for correlated ion motion and force an increase in noise. Surprisingly, the noise in the K₁₀₀ blockades was suppressed (FIGS. 24b and the black box in 24e) and the S_{l/f} amplitude was nearly collinear (FIG. 24e; black dashed line) with the normalized noise pore measured at much lower currents in the same pore. Without increased noise, it was inferred from this data that Na⁺ ions must not carry the blockade current because even the unhydrated ion was too large to translocate through the unoccluded volume. It was reasoned that the Grotthuss mechanism by which an excess proton defect diffuses or tunnels along the hydrogen-bonded water network inside a pore could account for the blockade current, even without the translocation of a proton. This proposition was tested in several ways by: 1. substituting MgCl₂ electrolyte (unhydrated Mg²⁺ has a smaller ionic radius-0.072 nm) in place of NaCl, which resulted in practically no change in the fluctuation amplitude CV=0.08, ΔIᵣₘₛ=22 pA, and 2. lowering the pH from 7 to 3.3 (FIG. 24e; black dot), which resulted in an increase in the fluctuation amplitude CV=0.155, ΔIᵣₘₛ=7.5 pA. With this evidence, it was speculated that proton or proton defects carry the blockade current and so, it seems reasonable that the read fidelity could be improve by varying the electrolyte constituency and pH.

Low fidelity reads with multiple monomers affecting the blockade current should not pose an insurmountable problem for sequencing protein, provided that the translocation rate is controlled if the coverage is high. Increasing the coverage by averaging reads from clusters of blockades from the same protein results in significant noise reduction and increased accuracy. Thus, to benchmark bioinformatics methods for calling reads, initially clusters of blockades acquired from pure protein solutions will be used.

Two machine learning candidates will be tested: 1. an ensemble tree-based method such as MART (Multiple Additive Regression Trees)¹⁴ or random forest (RF);^{9,80,81} and 2. an L1-penalized LASSO logistic regression.⁸² On the one hand, the hidden Markov model (HMM) is well suited to the turnstile-like format of the data and has the advantage of decoding the whole sequence,¹⁵ but the prima fad complexity of the algorithm seems unwieldy for protein, as the state-space grows geometrically with the number of AAs and PTMs. Although feasible algorithmically, an HMM with 20⁴=160,000-states would require an extensive training. However, redesigning a naïve HMM by combining the multiple states into a single one, e.g., representing each AA composition as a single state, is feasible. On the other hand, regression-based methods can incorporate many features to achieve similar power to HMMs with less of a computational burden. For example, RF-models are widely used in bioinformatics because there are no distributional assumptions; non-linear interactions and classification boundaries can be easily handled; they are robust to outliers; and there is reduced over-fitting. These advantages are offset by their limited interpretative power, however. In comparison, LASSO does variable selection automatically and offers an easily interpretable model; it is preferred over tree-based methods. Regression-based methods not only give the most-likely call for each fluctuation, but also the likelihood of each AA. In particular, the least absolute shrinkage and selection operator LASSO model improves the accuracy and interpretability by altering the fitting process to select a subset of the provided covariates, which could be important for interpreting current blockades since the clusters may be contaminated by the outliers.

Random Forest (RF−) regression: As a pilot, an RF-model was benchmarked using five human proteins: H3.2, H3.3, H4, CCL5 and the H3N tail peptide. The proteins were split into three pairs: (CCL5, H3 tail), (H4, H3.2) and (H3.3, H3.2). For each pair, the model was trained using the protein with the higher number of blockades and the accuracy of identification was estimated using the other protein. The first two pairs represented proteins that were very different in length and AA composition, thus minimizing the over-fitting. The third pair were histones of the same length, differing by four AA substitutions. The RF-model was compared with the naïve volume model, which assumes that each fluctuation in the blockade corresponds to quadromer read. Initially, each quadromer qᵢ from the training set was converted to a feature vector fᵢ, where each element of the vector, consisted of a volume. (Later it was expanded to include a pairing of volume and hydrophilicity of each AA.) Assuming that the blockade current does not depend on the order, the training sets were expanded by randomly permuting the AAs in each fᵢ vector, while maintaining the corresponding qᵢ value. In contrast with the volume model, the RF-model was more robust to outliers with less over-fitting and so the features were defined by the volumes of all twenty AAs.

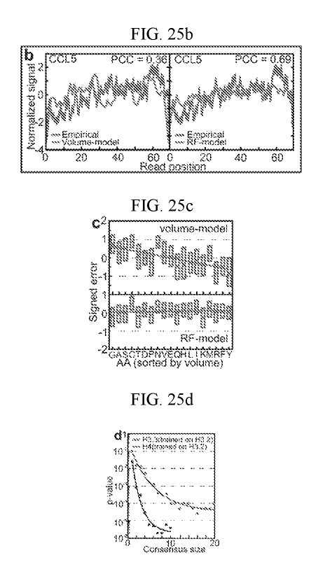

The RF-model performed well on the training sets and demonstrated significant improvement over the volume model as measured by the PCC (FIGS. 25a,b). Moreover, an analysis of error patterns revealed a bias in the signal estimation that was correlated with the volume (and hydrophilicity, not shown) of the AA. For each model, the bias was estimated by calculating the mean difference between the empirical and theoretical blockades (FIG. 25c). The volume model showed a bias indicating that AAs with a larger volume have a disproportional influence on the blockade current, whereas the RF-model showed no such bias. The volume model also showed a bias with respect to AA hydrophilicity, but not the RF-model. This Searches based on TD-MS data⁸³⁻⁸⁵ against small proteomes indicates reliable identification of proteins can be accomplished with p-values between 10⁻⁴ and 10⁻⁶. Commensurately, the analysis of the data acquired from a sub-nanopore with the RF regression revealed similar p-values for identifying single protein molecules in small proteomes (FIG. 25d). In particular, using the RF-model, H3.3 was identified (p<10⁻⁵) for a five-blockade cluster after training on a decoy H3.2 dataset of size 5×10⁶, consistently out-performing the volume model. The improved performance on the proteins that were more similar to the training proteins highlights the importance of the training set. Finally, the RF-model was benchmarked using a database extracted from the human proteome, consisting of all proteins ranging from 100 to 160 AAs in length (14,293 proteins). An H3.3 consensus was identified and ranked fifth against all other proteins (for a cluster of five).

Risk Mitigation: To improve the interpretive power of the RF-model, a weighted neighborhood scheme will be implemented, starting from a training set {(xₗ,yₗ), . . . , (xₙ,yₙ)} and making a prediction y for a new point x by analyzing the “neighborhood” of the point weighted by a weight function w, such that y=Σw(xᵢ)·yᵢ. Although it is possible to cluster reads based on the number of AAs, proteins may have similar length. By applying clustering algorithms like Affinity Propagation, which automatically estimates the number of clusters, a finer sub-partition of proteins with similar length can be generated resulting in reduced E-values. Since the E-value is the product of the p-value and the database size, a cluster of ten results in a 10-fold improvement.

Sequencing protein with a sub-nanopore as proposed in the present methods provides for revealing the primary structure of a protein as well as the diversity of the proteome. It will do so with single molecule sensitivity and a footprint about the size of flash drive. The present disclosure and techniques described accommodates a complex interplay between biology, chemistry, physics, statistics and computer science to protein and peptide analysis and characterization and new drug discovery techniques.

It is specifically intended that the present invention not be limited to the embodiments and illustrations contained herein, but include modified forms of those embodiments including portions of the embodiments and combinations of elements of different embodiments as come within the scope of the following claims.

### BIBLIOGRAPHY

The following references are specifically incorporated herein by reference.
1. A. F. M. Altelaar, J. Munoz, A. J. R. Heck, Nat. Rev. Genet., 14, 35-48 (2013).
2. L. Brocchieri and S. Karlin, Nuc. Acids. Res., 33(10), 3390-3400 (2005).
3. E. Kennedy, Z. Dong, C. Tennant, G. Timp, submitted to Nat. Nanotech. (2015).
4. C. Tasserit, et al., Phys. Rev. Lett. 260602 (2010).
5. J-I Nakayma, et. al. Science, 292 (5514), 110-113 (2001).
6. http://www.uniprot.org/unirprot/Q71DI3 (H32 Human); http://www.uniprot.org/unirprot/P01861 (IGHG4_Human).
7. N. Bandeira, et al., Nat Biotechnol. 26(12), 1336-133 (2008).
8. R. C. Aalberse and J. Schuurman, Immunology, 105, 9-19 (2002).
9. J. A. Reynolds and C. Tanford. Proc. Natl. Acad. Sci. USA. 66, 1002-1003 (1970).
10. K. L. Gudiksen, et. al., Biophys. J., 91, 298-310 (2006).
11. C. Ho, et al., Proc. Natl. Acad. Sci. USA, 102, 10445-10450 (2005).
12. J. Barthel, “Dr. Probe—High-resolution (S)TEM image simulation software,” July 2015. URL http://www.er-c.org/barthel/drprobe/
13. H. Ohtaki and T. Radnai, Chem. Rev. 93, 1157-1204 (1993).
14. E. M. Nelson, H. Li and G. Timp, ACS Nano, 8(6), 5484-5493 (2014).
15. D. S. Talaga and J. Li, I Am. Chem. Soc., 131, 9287-9297 (2009).
16. M. Carrion-Vazquez, et. al., Proc. Natl. Acad. Sci. USA, 96, 3694-3699 (1999).
17. B. Cheng, et. al., Nanoscale, 2015, 7, 2970-2977 (2015).
18. H. Lu and K. Schulten, Chemical Physics, 247, 147-153 (1999).
19. Tianxiang Su, Prashant K. Purohit, Acta Biomaterialia 5, 1855-1863 (2009).
20. K. E. Malek and R. Szoszkiewicz, J Biol Phys., 40, 15-23 (2014).
21. S. J. Perkins, Eur. J. Biochem. 157, 169-180 (1986).
22. W. Timp, J. Comer, A. Aksimentiev, Biophys. J., 102, L37-39 (2012).
23. V. Dimitrov, U. Mirsaidov, D. Wang, T. Sorsch, W. Mansfield, J. Miner, F Klemens, R. Cirelli, S. Yemenicioglu and G Timp, Nanotechnology, 21 065502 (2010).
24. R. M. M. Smeets, U. F. Keyser, N. H. Dekker and C. Dekker, Proc. Natl Acad. Sci. USA 105, 417-21 (2008).
25. S. Kim, S. H. Park, C. S. Ryu, Physics. Lett. A, 236, 409-414 (1997).
26. J. Luczka, T. Czernik, and P. Hänggi, Phys. Rev. E, 56(4), 3968-3975 (1997).
27. P. Hangii, Chem. Phys. Chem., 3, 285-290 (2002).
28. J. L. Hutter, J. Bechhoefer, Rev. Sci. Instr., 64, 1868-1873 (1993).
29. M. de Odrowaz Piramowicz, P. Czuba, M. Targosz, K. Burda, M. Szymonski, Acta Biochim. Pol., 53, 93-100 (2006).
30. E. L. Florin, V. T. Moy, H. E. Gaub, Science, 264, 415-417 (1994).
31. V. Kurz, E. M. Nelson, J. Shim, G. Timp, ACS Nano 7, 4057-4069 (2013).
32. D. C. Grahame, Chem. Rev., 41, 441-501 (1947).
33. M. Wilhem, J. Schlegl, H. Hahne, A. M. Gholami, M. Lieberenz, E. Ziegler, L. Butzmann, S. Gessulat, H. Marx, T. Mathieson, S. Lemeer, K. Schnatbaum, U. Reimer, H. Wenschuh, M. Mollenhauer, J. Slotta-Huspennina, J-H. Boese, M. Bantscheff, A. Gerstmair, F. Faerber and B. Kuster, Nature 509, 582-587 (2014).
34. A. F. M. Altelaar, J. Munoz, A. J. R. Heck, Nat. Rev. Genet., 14, 35-48 (2013).
35. K. Chandramouli and P-Y. Qian, Human Genomics and Proteomics, Article ID 239204, doi:10.4061/2009/239204 (2009).
36. T. Ohshiro, et. al., Nature Nanotech. 9, 835-40 (2014).
37. L. Movileanu, et. al., Nat. Biotechnol. 18, 1091-1095 (2000).
38. M. M. Mohammad, et. al., J. Am. Chem. Soc. 130, 4081-4088 (2008).
39. D. S. Talaga and J. Li, J. Am. Chem. Soc., 131, 9287-9297 (2009).
40. R. Wei, V. Gatterdam, et. al., Nat. Nanotechnol., 7, 257-263 (2012).
41. K. J. Freedman, et. al., Anal. Chem,. 83(13), 5137 (2011).
42. D. Fologea, B. Ledden, D. S. McNabb, and J. Li, Appl. Phys. Lett., 91(5), 053901 (2007).
43. B. Cressiot, A. Oukhaled, G. Patriarche, M. Pastoriza-Gallego, J.-M. Betton, L. Auvray, M. Muthukumar, L. Bacri, and J. Pelta, ACS Nano, 6, 6236-6243 (2012).
44. E. M. Nelson, V. Kurz, J. Shim, W. Timp, and G. Timp, Analyst, 137(13), 3020-3027 (2012).
45. J. Nivala, D. B. Marks, M. Akeson, Nat. Biotechnol., 31, 247-250 (2013)
46. D. Rodriguez-Larrea, H. Bayley, Nat. Nanotechnol., 8, 288-295 (2013).
47. C. B. Rosen, D. Rodriguez-Larrea, and H. Bayley, Nat Biotechnol., 32 (2), 179-81 (2014).
48. C. Merstorf, B. Cressiot, M. Pastoriza-Gallego, A. Oukhaled, J.-M. Betton, L. Auvray, J. Pelta, ACS Chem. Biol., 7, 652-658 (2012).
49. J. Nivala, L. Mulroney, G. Li, J. Schreiber, and M. Akeson, ACS Nano, 8(12), 12365-12375 (2014).
50. H. Laszlol, et. al., Nat. Biotech., 32(8), 829-833 (2014).
51. M. Jain, I. T. Fiddes, K. H. Miga, H. E. Olsen, B. Paten and M. Akeson, Nature Methods 12, 351-356 (2015).
52. W. Timp, A. M. Nice, E. M. Nelson, V. Kurz, K. McKelvey, G. Timp, IEEE Access 2, 1396-1408 (2014).
53. J. Li, D. Fologea, R. Rollings, and B. Ledden, Protein Pep. Lett., 21(3), 256-265 (2014).
54. G. Sigalov, J. Comer, G. Timp, A. Aksimentiev, Nano Lett. 8, 56-63 (2008).
55. C. Ho, Qiao, J. B. Heng, A. Chatterjee, R. J. Timp, N. R. Alum, and G. Timp, Proc. Natl. Acad. Sci. USA, 102, 10445-10450 (2005).
56. H. Ohtaki and T. Radnai, Chem. Rev. 93, 1157-1204 (1993).
57. J. A. Reynolds and C. Tanford, Proc. Natl. Acad. Sci. USA. 66, 1002-1003 (1970).
58. K. Ibel, R. P. May, K. Kirschner, H. Szadkowski, E. Mascher, and P. Lundahl, Eur. J. Biochem. 190, 311-318 (1990).
59. M. Samso, J.-R. Daban, S. Hansen and G. R. Jones, Eur. J. Biochem., 232, 818-824 (1995).
60. W. L. Mattice, J. M. Riser, and D. S. Clark, Biochemistry. 15, 4264-4272 (1976).
61. P. Lundahl, E. Greijer, M. Sandberg, S. Cardell, and K. O. Eriksson, Biochim. Biophys. Acta. 873:20-26 (1986).
62. K. L. Gudiksen, I. Gitlin, D. T. Moustakas, and G. M. Whitesides, Biophys. J, 91, 298-310 (2006).
63. W. H. J. Westerhuis, J. N. Sturgis, and R. A. Niederman, Anal. Biochem., 284, 143-152 (2000).
64. http://www.uni prot.org/unirprot/P13501 (CCL5_Human); http://www.uni prot.org/unirprot/P02769 (Albumin_Bovine); http://www.uni prot.org/unirprot/P09341 (GROA_Human); and http://www.uni prot.org/unirprot/P68431 (H 3_Human)
65. Shortle and M. S. Ackerman, Science, 293, 487-489 (2001).
66. H. Bayley and P. S. Cremer, Nature 413, 226-230 (2001).
67. G. J. Szekely and M. L. Rizzo. J. Multivariate Analysis, 93(1), 58-80 (2005). doi:10.1016/j.j mva2003.12.002.
68. E. M. Nelson, H. Li and G. Timp, ACS Nano, 8(6), 5484-5493 (2014).
69. S. J. Perkins, Eur. J. Biochem. 157, 169-180 (1986).
70. W. Timp, J. Comer, A. Aksimentiev, Biophys. J., 102, L37-39 (2012).
71. J. Friedman, H. T. Hastie and R. Tibshirani, J. Statistical Software, 33.1 (2010): 1.
72. Y. Oma, Y. Kino, N. Sasagawa, S. Ishiura,./. Biol. Chem., 279, 21217 (2004).
73. J-I Nakayma, et. al., Science, 292 (5514), 110-113 (2001).
74. Z. Wang, C. Zang, J. A. Rosenfeld, D. E. Schones, A. Barski, S. Cuddapah, K. Cui, T-Y. Roh, W. Peng, M. Q Zhang and K. Zhao, Nature Genetics, 40, 897-903 (2008).
75. J. Barthel. Dr. Probe—High-resolution (S)TEM image simulation software, July 2015. URL http://www.er-c.org/barthel/drprobe/
76. J. M. Cowley and A. F. Moodie, Acta. Cryst., 10, 609-619 (1957).
77. Weickenmeier and H. Kohl, Acta Cryst., A 47, 590-597 (1991),
78. Raillon, P. Granjon, M. Graf, L. J. Steinbocka, A. Radenovic, A., 2012. Nanoscale. 4: 4916-4924 (2012).
79. V. Kurz, E. M. Nelson, J. Shim, G. Timp, ACS Nano 7, 4057-4069 (2013).
80. D. C. Grahame, Chem. Rev., 41, 441-501 (1947).
81. G. Gergious, et. al., Nat. Biotech. 32(2), 158-168 (2014).
82. V. Greiff, U. Menzel, U. Haessler, S. C. Cook, S. Friedensohn, T. A. Khan, M. Pogson, I. Hellmann and S. T. Reddy, BMC Immunology, 15, 40 (2014). http://www.biomedcentral.com/1471-2172/40
83. N. Bandeira, et. al., Nat Biotechnol. 26(12), 1336-133 (2008).
84. D. R. Boutz, A. P. Horton, Y. Wine, J. J. Lavinder, G. Georgiou, and Edward M. Marcotte, Analytical Chem. (2013). dx.doi.org/10.1021/ac4037679
85. M. Wilhem, et al., Nature 509, 582-587 (2014).
86. A. F. M. Altelaar, J. Munoz, A. J. R. Heck, Nat. Rev. Genet., 14, 35-48 (2013).
87. K. Chandramouli and P-Y. Qian, Human Genomics and Proteomics, Article ID 239204, doi:10.4061/2009/239204 (2009). L. Movileanu, S. Howorka, O. Braha, and H. Bayley, Nat. Biotechnol. 18, 1091-1095 (2000).
88. N. Fischer, “Sequencing antibody repetoirs”, mAbs 3(1), 17-20 (2011).
89. M. M. Mohammad, et. al., J. Am. Chem. Soc. 130, 4081-4088 (2008).
90. D. S. Talaga and J. Li, J. Am. Chem. Soc., 131, 9287-9297 (2009).
91. R. Wei, et. al., Nat. Nanotechnol., 7, 257-263 (2012).
92. K. J. Freedman, et. a., Anal. Chem., 83(13), 5137 (2011).
93. D. Fologea, B. Ledden, D. S. McNabb, and J. Li, Appl. Phys. Lett., 91(5), 053901 (2007).
94. B. Cressiot, et. al., ACS Nano, 6, 6236-6243 (2012).
95. E. M. Nelson, V. Kurz, J. Shim, W. Timp, and G. Timp, Analyst, 137(13), 3020-3027 (2012).
96. J. Nivala, D. B. Marks, M. Akeson, Nat. Biotechnol., 31, 247-250 (2013)
97. D. Rodriguez-Larrea, H. Bayley, Nat. Nanotechnol., 8, 288-295 (2013).
98. C. B. Rosen, D. Rodriguez-Larrea, and H. Bayley, Nat Biotechnol., 32 (2), 179-81 (2014).
99. C. Merstorf, et. al., ACS Chem. Biol., 7, 652-658 (2012).
100. J. Nivala, et. al., ACS Nano, 8(12), 12365-12375 (2014).
101. E. Kennedy, Z. Dong, C. Tennant, G. Timp, submitted to Nat. Nanotech. (2015).
102. H. Ohtaki and T. Radnai, Chem. Rev. 93, 1157-1204 (1993).
103. R. G. Hamilton, Asthma and Allergy Center, Johns Hopkins University School of Medicine (2001).
104. R. C. Aalberse and J. Schuurman, Immunology, 105, 9-19 (2002).
105. T. Ohshiro, M. Tsutsui, K. Yokota, M. Furuhashi, M. Taniguchi and T. Kawai, Nature Nanotech. 9, 835-40 (2014).
106. U.S. Pat. No. 5,795,782 (1998)—Church.
107. A. H. Laszlo, et. al., Nat. Biotech., 32(8), 829-833 (2014).
108. M. Jain, et. al., Nature Methods 12, 351-356 (2015).
109. N. J. Loman, J. Quick, and J. T. Simpson, Nat Meth, 12(8), 733-735 (2015).
110. W. Timp, A. M. Nice, E. M. Nelson, V. Kurz, K. McKelvey, G. Timp, IEEE Access 2, 1396-1408 (2014).
111. M. Shankla and A. Aksimentiev, Nat. Commun., (2014). DOI: 10.1038/ncomms6171.
112. S. Carson and M. Wanunu, Nantechnology, 26, 074004 (2015). DOI: 10.1088/0957-4484/26/7/074004
113. J. Li, D. Fologea, et. al., Protein Pep. Lett., 21(3), 256-265 (2014).
114. G. Sigalov, J. Comer, G. Timp, A. Aksimentiev, Nano Lett. 8, 56-63 (2008).
115. C. Ho, et. al., Proc. Natl. Acad. Sci. USA, 102, 10445-10450 (2005).
116. J. A. Reynolds and C. Tanford, Proc. Natl. Acad. Sci. USA, 66, 1002-1003 (1970).
117. K. Ibel, R. P. May, K. Kirschner, H. Szadkowski, E. Mascher, and P. Lundahl, Eur. J. Biochem., 190, 311-318 (1990).
118. M. Samso, J.-R. Daban, S. Hansen and G. R. Jones, Eur. J. Biochem., 232, 818-824 (1995).
119. W. L. Mattice, J. M. Riser, and D. S. Clark, Biochemistry, 15, 4264-4272 (1976).
120. P. Lundahl, E. Greijer, M. Sandberg, S. Cardell, and K. O. Eriksson. Biochim. Biophys. Acta. 873:20-26 (1986).
121. K. L. Gudiksen, I. Gitlin, D. T. Moustakas, and G. M. Whitesides, Biophys. J., 91, 298-310 (2006).
122. W. H. J. Westerhuis, J. N. Sturgis, and R. A. Niederman, Anal. Biochem., 284, 143-152 (2000).
123. http://www.uniprot.org/unirprot/P13501 (CCL5_Human); http://www.uniprot.org/unirprot/P02769 (Albumin_Bovine); http://www.uniprot.org/unirprot/P09341 (GROA_Human); and http://www.uniprot.org/unirprot/P68431 (H3_Human) http://www.uniprot.org/unirprot/P01861 (IGHG4_Human).
124. D. Shortle and M. S. Ackerman, Science, 293, 487-489 (2001).
125. T. Hastie, R. Tibshirani and J. Friedman, Chapter 6, Springer (2011).
126. W. S. Cleveland, S. J. Devlin, J. Am. Stat. Assoc., 83 (403), 596-610 (1988).
127. E. M. Nelson, H. Li and G. Timp, ACS Nano, 8(6), 5484-5493 (2014).
128. M. Carrion-Vazquez, A. F. Oberhauser, S. B. Fowler, P. E. Marszalek, S. E. Broedel, J. Clarke, and J. M. Fernandex, Proc. Natl. Acad. Sci. USA, 96, 3694-3699 (1999).
129. B. Cheng, S. Wu, S. Liu, P. Rodriguez-Aliaga, J. Yu and S. Cui, Nanoscale, 7, 2970-2977 (2015).
130. H. Lu and K. Schulten, Chem. Physics, 247, 147-153 (1999).
131. T. Su, P. K. Purohit, Acta Biomaterialia 5, 1855-1863 (2009).
132. K. E. Malek and R. Szoszkiewicz, J Biol Phys., 40, 15-23 (2014).
133. J. Wilson, L. Sloman, and A. Aksimentiev, (2015) unpublished.
134. A. K. Geim, K. S. Novoselov, Nat. Mater., 6, 183-191 (2007).
135. M Munz, C. E. Giusca, R. L. Myers-Ward, D. K. Gaskill, and O. Kazakova, ACS Nano, 9(8), 8401-8411 (2015).
136. G. F. Schneider, S. W. Kowalczyk, V. E. Calado, G. Pandraud, H. W. Zandbergen, L. M. Vandersypen, C. Dekker, Nano Lett. 10, 3163-3167 (2010).
137. Z. Li, Y. Wang, A. Kozbial, G. Shenoy, F. Zhou, R. McGinley, P. Ireland, B. Morganstein, A. Kunkel, S. P. Surwade, L. Li and H. Liu, Nat. Mater., 12, 925-931 (2013).
138. D. B. Mahadik, et. al., J. Colloid Interface Sci., 356, 298-302 (2011).
139. M. Zhou, et. al., J. Mater. Chem., 21, 693-704 (2011).
140. http://www.nanocs.net/PEG/Surface-reactive-PEG/methoxy-PEG-silane/Silane-PEG-550.htm.
141. S. J. Perkins, Eur. J. Biochem. 157, 169-180 (1986).
142. Y. Oma, Y. Kino, N. Sasagawa, S. Ishiura, J. Biol. Chem., 279, 21217 (2004).
143. A. B. Farimani, K. Min, N. R. Alum, ACS Nano, 8(8), 7914-7922 (2014).
144. J. Li, private communications.
145. W. Timp, J. Comer, A. Aksimentiev, Biophys. J., 102, L37-39 (2012).
146. J. H. Friedman, Annals Stat., 1189-1232 (2001).
147. J. Friedman, H. T. Hastie and R. Tibshirani, J, Statistical Software, 33.1 (2010): 1.
148. J. Schreiber and K. Karplus, Bioinformatics, 31, (12) 1897-1903 (2015).
149. J. D. Thompson, et. al., Nucleic Acids Res., 27(13), 2682-90 (1999).
150. D. J. Lipman, et. al., Proc. Natl. Acad. Sci. USA, 86, 4412-4415 (1989).
151. Q. Dua, D. Wei, K-C. Chou, Peptides, 24 1 863-1 869 (2003).
152. Z. Dosztanyi and A. E. Torda, Bioinformatics, 17(8) 686-699 (2001).
153. David S. Horner, et al., Brief Bioinformatics, 9(1) 46-56 (2007).
154. A. Afek, E. I. Shakhnovic, and D. B. Lukatsky, J. Mol. Biol. 409(3), 439-49 (2011). DOI:10.1016/j.jmb.2011.03.056.
155. H. Tilgner, et. al., PNAS, 111 (27) 9869-9874 (2014).
156. P. A. Larsen and T. P. L. Smith, BMC Immunology, 13 (1), 52 (2012).
157. J. A. Weinstein, X. Zeng, Y. H. Chien, S. R. Quake, PLoS ONE 8, e67624 (2013).
158. Kinde, J., et. al., Proc. Nat. Acad. Sci. USA, 108(23), 9530-9535 (2011).

We claim:

1. A thin membrane comprising an inorganic material, said thin membrane comprising a surface with a defined topography comprising nanopores, said nanopores having a diameter of between about 0.3 nm to about 1.5 nm, wherein said thin membrane has a thickness of about t=8 nm to 12 nm.

2. The thin membrane of claim 1 wherein said inorganic material is silicon nitride and the pores are other than MspA pores.

3. The thin membrane of claim 1 wherein the nanopores are sub-nanopores having a diameter of less than 1,000 pm.

4. The thin membrane of claim 1 wherein the nanopores are electron beam sputtered onto the thin membrane to provide a defined biconical topography on the membrane surface.

5. The thin membrane of claim 1 wherein the defined topography of the surface comprises a biconical configuration having cone angles in a range of about θ=15+/−5°.

6. The thin membrane of claim 2 wherein said membrane is resistant to denaturant detergent and temperatures between 45° to 100° C.

7. A thin membrane-silicon chip construct comprising:
a silicon chip; and
the thin membrane of claim 1,
wherein the thin membrane is plasma bonded to a surface of the silicon chip.

8. The thin membrane-silicon chip construct of claim 7 wherein the nanopores are sub-nanopores having a diameter of less than 1,000 pm.

9. A method for identifying an amino acid within an amino acid sequence of a molecule of interest, said method comprising:
denaturing the amino acid sequence of the molecule of interest to provide a denatured amino acid containing preparation;
depositing said denatured amino acid containing preparation onto a surface of a thin inorganic membrane, said inorganic membrane surface comprising nanopores with a size of about 0.3 nm to about 1.5 nm, to provide a membrane having amino-acid associated nanopores;
wetting the membrane surface with an electrolyte solution to provide a wetted membrane surface;
translocating the amino acid associated with nanopores of the surface by applying a transmembrane current voltage to the membrane in the presence of an electrolyte solution,
identifying the amino acid of the amino acid sequence by determining a pore current value, said pore current value comprising a measure of the fluxuations in the electronic current associated with impelling the amino acid thought the nanopore.

10. The method of claim 9 wherein the electrolyte solution is an NaCl solution

11. The method of claim 9 wherein the NaCl solution is a 200-300 mM NaCl solution.

12. The method of claim 9 wherein the amino acid containing molecule of interest is a protein.

13. The method of claim 12 wherein the nanopore is not an MspA pore.

14. The method of claim 12 wherein the protein is an antibody.

15. The method of claim 9 wherein the nanopores are sub-nanopores having a diameter of less than 1,000 pm.

16. The method of claim 15 wherein the thin inorganic membrane is wetted with the electrolyte solution for about 24 hours prior to applying the transmembrane voltage to the membrane.

17. The method of claim 12 wherein the protein comprises an amino acid length of about to about 3 amino acids to about 300 amino acids.

18. The method of claim 9 wherein the inorganic membrane is a transmembrane voltage is applied using Ag/Al electrodes.

19. The method of claim 15 wherein the sub-nanopores have a diameter of about 0.3 nm to about 0.9 nm.

20. The method of claim 9 wherein the thin inorganic membrane comprises a thin inorganic silicon nitride membrane having a defined conical topography, and wherein said nanopores are provided on said membrane surface with an electron beam sputtering technique to provide nanopores.
